(19) United States
(12) Patent Application Publication
(10) Pub. No.: US 2025/0197875 A1
(43) Pub. Date: Jun. 19, 2025

(54) ENGINEERED PLANTS HAVING MODIFIED INOSITOL PYROPHOSPHATES

(71) Applicant: Virginia Tech Intellectual Properties, Inc., Blacksburg, VA (US)

(72) Inventors: Glenda Gillaspy, Blacksburg, VA (US); Catherine Freed, Blacksburg, VA (US); Caitlin Cridland, Blacksburg, VA (US); Branch Craige, Blacksburg, VA (US)

(21) Appl. No.: 18/847,106

(22) PCT Filed: Mar. 14, 2023
(86) PCT No.: PCT/US2023/064331

§ 371 Date: Sep. 13, 2024

Related U.S. Application Data
(60) Provisional application No. 63/319,679, filed on Mar. 14, 2022.

(51) Int. Cl. C12N 15/82; C12N 9/12

(52) U.S. Cl. CPC C12N 15/8243; C12N 9/1205; C12N 9/1229; C12Y 207/01134; C12Y 207/04024

(57) ABSTRACT

Described in certain example embodiments herein are engineered plants that overexpress an ITPK and/or a VIP gene, gene product, or both. In some embodiments, the VIP gene, gene product, or both is the kinase domain of the an ITPK gene product. Also described in certain example embodiments herein are methods of making and uses of the engineered plants described herein.

### CROSS-REFERENCE TO RELATED APPLICATIONS

This application claims the benefit of and priority to co-pending U.S. Provisional Patent Application No. 63/319,679, filed on Mar. 14, 2022, entitled “ENGINEERED PLANTS HAVING MODIFIED INOSITOL PYROPHOSPHATES”, the contents of which is incorporated by reference herein in its entirety.

### STATEMENT REGARDING FEDERALLY SPONSORED RESEARCH

This invention was made with government support under Grant No. MCB 161038 awarded by National Science Foundation. The government has certain rights in the invention.

### SEQUENCE LISTING

This application contains a sequence listing filed in electronic form as an xml file entitled VTIP-0375WP_ST26.xml, created on Mar. 13, 2023 and having a size of 40,687. The content of the sequence listing is incorporated herein in its entirety.

### TECHNICAL FIELD

The subject matter disclosed herein is generally directed to engineered plants with improved phosphorus-use efficiency (PUE) and uses thereof.

### BACKGROUND

Phosphorous is a crucial macronutrient necessary for plant growth and development and is a major limiting factor in crop yield in the developed world. Plants uptake phosphate in the form of inorganic phosphate (Pi), however, Pi is often limited in agricultural soils. In addition, Pi is a non-renewable resource with current reserves projected to be depleted in as early as 30 years (Cordell & White 2011; Herrera-Estrella & López-Arredondo 2016). As such, there is a need for plants, compositions, and methods for improved utilization of environmental phosphorous.

Citation or identification of any document in this application is not an admission that such a document is available as prior art to the present invention.

### SUMMARY

Described in certain example embodiments herein are engineered plant comprising: increased inositol pyrophosphates and/or synthesis thereof as compared to a wild-type or unmodified plant. In certain example embodiments, the engineered plant comprises increased expression and/or amount of (a) an inositol tetrakisphosphate kinase (ITPK) gene and/or gene product; (b) a diphosphoinositol pentakisphosphate (VIP) gene and/or gene product; or (c) both (a) and (b).

In certain example embodiments, the ITPK gene and/or gene product is an inositol tetrakisphosphate 1-kinase 1 (ITPK1) gene and/or gene product or an inositol tetrakisphosphate kinase 2 (ITPK2) gene and/or gene product. In certain example embodiments, the VIP gene and/or gene product is a inositol diphosphoinositol pentakisphosphate 1 (VIP1) gene and/or gene product or an inositol diphosphoinositol pentakisphosphate 2 (VIP2) gene and/or gene product.

In certain example embodiments, (a) the VIP gene and/or gene product comprises or consists of the kinase domain of dual domain diphosphoinositol pentakisphosphate kinase 1 (VIP1KD) and/or the kinase domain of the dual domain diphosphoinositol pentakisphosphate kinase 2 (VIP2KD), (b) the ITPK gene and/or gene product comprises or consists of the kinase domain of the inositol tetrakisphosphate 1-kinase 1 (ITPK1KD) and/or the kinase domain of the inositol tetrakisphosphate kinase 2 (ITPK2KD).

In certain example embodiments, the increase in expression and/or amount of ITPK1 gene and/or gene product, VIP gene and/or gene product, and/or the one or more gene and/or gene products thereof associated with the phosphate starvation response is 1-1,000 fold or more as compared to a wild-type or unmodified plant.

In certain example embodiments, the engineered plant has increased phosphorous use efficiency as compared to a wild-type or unmodified plant. In certain example embodiments, the phosphorous use efficiency is increased 1-1,000 fold or more as compared to a wild-type or unmodified plant.

In certain example embodiments, the engineered plant has reduced Pi accumulation capability and/or capacity as compared to a wild-type or unmodified plant. In certain example embodiments, the Pi accumulation capability and/or capacity is reduced by 1-1,000 fold or more as compared to a wild-type or unmodified plant.

In certain example embodiments, the engineered plant has one or more modified developmental pathways and/or hormone signaling pathways.

In certain example embodiments, the engineered plant comprises one or more modifications to (a) an endogenous ITPK1 gene and/or gene product; (b) an endogenous VIP gene and/or gene product; or (c) both (a) and (b), wherein the one or more modifications increases the expression of an ITPK gene and/or gene product and/or a VIP gene and/or gene product as compared to a wild-type or unmodified plant or cell(s) thereof.

In certain example embodiments, the one or more modifications comprise an insertion of one or more nucleotides, a deletion of one or more nucleotides, a substitution of one or more nucleotides, or any combination thereof in the endogenous ITPK gene and/or gene product and/or endogenous VIP gene and/or gene product.

Described in certain example embodiments herein are methods of producing an engineered plant and/or increasing phosphorus use efficiency in a plant comprising: overexpressing, in a plant and/or one or more plant cells (a) an inositol tetrakisphosphate kinase (ITPK) gene and/or gene product; (b) a diphosphoinositol pentakisphosphate (VIP) gene and/or gene product; or (c) both (a) and (b). In certain example embodiments, the ITPK gene and/or gene product is an inositol tetrakisphosphate 1-kinase 1 (ITPK1) gene and/or gene product or an inositol tetrakisphosphate kinase 2 (ITPK2) gene and/or gene product. In certain example embodiments, the VIP gene and/or gene product is a inositol diphosphoinositol pentakisphosphate 1 (VIP1) gene and/or gene product or an inositol diphosphoinositol pentakisphosphate 2 (VIP2) gene and/or gene product

In certain example embodiments, overexpressing comprises introducing, into one or more cells of a plant, (a) an exogenous ITPK gene and/or gene product; (b) an exogenous VIP gene and/or gene product; or (c) both (a) and (b). In certain example embodiments, overexpression comprises introducing, into one or more cells of a plant, one or more modifications in (a) an endogenous ITPK gene and/or gene product; (b) an endogenous VIP gene and/or gene product; or (c) both (a) and (b), wherein the one or more modifications increases the expression of a ITPK1 gene and/or gene product, a VIP gene and/or gene product, one or more gene and/or gene products associated with the phosphate starvation response, or any combination thereof as compared to a wild-type or unmodified plant or cell(s) thereof.

In certain example embodiments, the one or more modifications comprise an insertion of one or more nucleotides, a deletion of one or more nucleotides, a substitution of one or more nucleotides, or any combination thereof in the endogenous ITPK gene and/or gene product and/or endogenous VIP gene and/or gene product.

Described in certain embodiments herein are engineered plants produced by a method described herein.

Described in certain example embodiments herein are methods comprising planting, growing, harvesting, and/or cultivating an engineered plant of the present invention or made by a method of the present invention in a growth medium, wherein the growth medium has low phosphorous, low phosphorus availability, or both. In certain example embodiments, the growth medium has low Pi, low Pi availability, or both. In certain example embodiments, the growth medium is soil or an aqueous environment. In certain example embodiments, planting, growing, harvesting, and/or cultivating comprises a reduced or eliminated application of an amount supplemental phosphorous and/or Pi as compared to planting, growing, harvesting, and/or cultivating a suitable control plant and/or suitable control planting, growing, harvesting, and/or cultivating conditions.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1G—Characterization and Comparison of ITPK1 OX and VIP2KD OX transgenics. (FIG. 1A) Western blot of 35-day-old rosette leaf tissue from WT and ITPK1 OX and VIP2KD OX transgenics. Ponceau. Staining shows RuBisCo accumulation in all plants as a positive control (˜55 kDa). (FIG. 1B) Arabidopsis rosette growth over the course of 50 days, each image representation of n=3 independent experiments containing 15 or more plants per genotype. Scale bar=1 cm. (FIG. 1C-1D) Average rosette diameter at 18-days (FIG. 1C) and 50-days (FIG. 1D). Each point represents an individual plant from two independent replicates where n=15 to 16 plants. (FIG. 1E) Time to flowering. Each point represents an individual plant from two independent replicates where n=12 to 32 plants. (FIG. 1F) Flowering phenotype of ITPK1, image representation of 3 independent experiments containing 15 to 16 plants. Blue arrow, primary inflorescence; black arrows, axillary inflorescences; pink arrows, secondary inflorescences. (FIG. 1G) Stick-and-ball model representing flowering phenotype of WT and ITPK1 OX. IM, inflorescence meristem; FM, floral meristem. Letters indicate statistical groups according to post-hoc Tukey HSD test (α=0.05), where different letters indicate statistically significant differences.

FIG. 2A-2E—InsP and PP-InsP Profiling of ITPK1 OX and VIP2KD OX Transgenics. (FIG. 2A-2E) All InsPs were extracted and separated using anion exchange HPLC, and the data were analyzed, as described in Experimental Procedures. (FIG. 2A-2B) InsP profiles are representative of n=2 to 3 independent replicates per genotype. (FIG. 2C) InsP₇/InsP₆, (FIG. 2D) InsP₈/InsP₇ and (FIG. 2E) InsP₈/InP₆ ratios. Letters indicate statistical groups according to post-hoc Tukey HSD test (α=0.05), where different letters indicate statistically significant differences.

FIG. 3—InsP and PP-InsP Profiling of ITPK1 OX and VIP2KD OX Transgenics. PAGE analysis of InsPs extracted from 4-week-old rosette tissue. 0.6 g of rosette tissue of WT and ITPK1 OX-1 were extracted as described in (Wilson et al. 2015, See Example 1). M, marker using seed extract from mrp5 seed that contains a mixture of InsP₆, InsP₇ and InsP₈. Orange G, the loading dye.

FIG. 4A-4C—Pi accumulation in ITPK1 OX and VIP2KD OX lines. (FIG. 4A-4B) Rosette leaf Pi accumulation in soil-grown plants at 24-days (FIG. 4A) and 50-days (FIG. 4B) of growth. (FIG. 4C) Green, yellow and purple rosette leaf Pi accumulation in soil-grown plants at 50-days of growth. Each point represents one biological replicate of pooled rosette leaf tissue from 3 to 5 plants; error bars show SD. Letters indicate statistical groups according to post-hoc Tukey HSD test (α=0.05), where different letters indicate statistically significant differences.

FIG. 5A-5F—PSR gene expression in ITPK1 OX and VIP2KD OX lines. (FIG. 5A-5F) Relative gene expression of SPX (FIG. 5A), PS2 (FIG. 5B), PHT1;4 (FIG. 5C), IPS1 (FIG. 5D), PHO1;H1 (FIG. 5E), and PHR1 (FIG. 5F) in 24-day (top) and 50-day (bottom) soil-grown plants. One biological replicate of pooled rosette leaf tissue from 3-5 plants; error bars show SE of technical replicates. Letters indicate statistical groups according to post-hoc Tukey HSD test (α=0.05), where different letters indicate statistically significant differences.

FIG. 6A-6C—(FIG. 6A-6C) Relative gene expression of SPX (FIG. 6A), PS2 (FIG. 6B), and PHT1;4 (FIG. 6C) in colored rosette leaves in 50-day soil-grown plants. Each point represents two biological replicates of pooled rosette leaf tissue from 3 to 5 plants; error bars show SE. Letters indicate statistical groups according to post-hoc Tukey HSD test (α=0.05), where different letters indicate statistically significant differences.

FIG. 7A-7B—JAZ9 expression in ITPK1 OX and VIP2KD OX lines. (FIG. 7A) JAZ9 expression (relative to WT expression of PEX4) in colored rosette leaves from undomed 50-day soil-grown plants. Each point represents two biological replicates of pooled rosette leaf tissue from 3 to 5 plants; error bars show SD. (FIG. 7B) JAZ9 expression (relative to WT expression of PEX4) in colored rosette leaves from domed 50-day soil-grown plants. Data from three technical replicates of one biological replicate of pooled rosette leaf tissue from 3 to 5 plants; error bars show SD. Letters indicate statistical groups according to post-hoc Tukey HSD test (α=0.05), where different letters indicate statistically significant differences.

FIG. 8—Potential model for Pi-sensing and Pi-responses when InsP8 levels are altered. When InsP₈ levels are low, SPX1 is unable to sequester PHR1, and PHR1 can bind to P1BS motifs, activating PSR gene expression. Induction of PSR genes leads to various responses included increased production of phosphate transporters (PHTs) and Pi uptake, lipid remodeling and anthocyanin production. When Pi levels are not limited, InsP₈ binds to SPX1 inducing a conformation change to allow binding with PHR1. PHO2, and NLA under certain conditions, can fine tune Pi uptake by targeting PHTs for degradation. In plants such as ITPK1 OX and VIP2KD OX that have elevated InsP₈, through an unknown mechanism, PSR genes are induced. Despite PHT gene induction, Pi accumulation levels remain low, suggestive of PHT degradation by PHO2 or SPX-domain-containing NLA.

FIG. 9A-9D—Growth and flowering characterization of ITPK1 OX and VIP2KD OX. (FIG. 9A-9B) Average rosette diameter at 24-days (FIG. 9A) and 35-days (FIG. 9B). Each point represents an individual plant from two independent replicates of n=15-16 plants. (FIG. 9C) Total cauline leaves at time of flowering. Each point represents an individual plant from two independent replicates of n=12-32 plants. (FIG. 9D) Time to bolting. Each point represents an individual plant from two independent replicates of n=12-32 plants.

FIG. 10A-10E—Leaf morphology characterization of ITPK1 OX and VIP2KD OX. (FIG. 10A-10D) Leaf blade length to leaf blade width ratio at 18-days (FIG. 10A), 24-days (FIG. 10B), 35-days (FIG. 10C), and 50-days (FIG. 10D). Leaf blade length and width was measured from the two leaves comprising the rosette diameter from each plant. (FIG. 10E) Cauline leaf phenotype of WT, ITPK1 OX-1 and VIP2KD OX-1 at 50-days. Each point represents an individual plant from two independent replicates of n=15-16 plants.

FIG. 11—Flowering phenotype of ITPK1 OX. Flowering phenotype of ITPK1 OX. Flowering phenotypes of ITPK1 OX-2 compared to age-match WT (right) and VIP2KD OX-2 (left) at 70-days.

FIG. 12A-12F—Root phenotype characterization of ITPK1 OX-1 and VIP2KD OX-1. (FIG. 12A-12B) Primary root length at 7-days (FIG. 12A), and 14-days (FIG. 12B). (FIG. 12C-12D) Number of lateral roots per seedling at 7-days (FIG. 12C), and 14-days (FIG. 12D). (FIG. 12E-12F) Lateral root length at 7-days (FIG. 12E), and 14-days (FIG. 12F). Each point represents an individual measurement from two independent replicates of n=19-33 seedlings.

FIG. 13A-13D—InsP and PP-InsP Profiling of itpk1. (FIG. 13A) All InsPs were extracted and separated using anion exchange HPLC, and data analyzed, as described in Experimental Procedures. InsP profiles are representative of 2-6 independent replicates per genotype. (FIG. 13B) InsP₇/InsP₆, (FIG. 13C) InsP₈/InsP₇ and (FIG. 13D) InsP₈/InP₆ ratios. Letters indicate statistical groups according to post-hoc Tukey HSD test (α=0.05).

FIG. 14—InsP and PP-InsP Levels of ITPK1 OX and VIP2KD OX Transgenics. PAGE analysis of InsPs extracted from 4-week-old rosette tissue. 0.6 g of rosette tissue of WT and ITPK1 OX-1 were extracted as described in (Wilson et al. 2015, See Example 1). 600 mg, InsPs extracted from 600 mg rosette tissue; 600 mg, InsPs extracted from 400 mg rosette tissue; 200 mg, InsPs extracted from 200 mg rosette tissue. M, marker using seed extract from mrp5 seed that contains a mixture of InsP₆, InsP₇ and InsP₈. Orange G, the loading dye.

FIG. 15A-15F—Inorganic Pi accumulation of ITPK1 OX and VIP2KD OX. (FIG. 15A-15D) Inorganic Pi accumulation of mixed rosette tissue at 14 days (FIG. 15A), 18 days (FIG. 15B), 35 days (FIG. 15C), and 70 days (FIG. 15D). (FIG. 15E) Inorganic Pi accumulation of senesced leaves at 70-days. Letters indicate statistical groups according to post-hoc Tukey HSD test (α=0.05). (FIG. 15F) Inorganic Pi accumulation in green rosette leaves, bolts, cauline leaves and flowers. Each point represents one biological replicate of pooled rosette leaf tissue from 3-5 plants; error bars show SD.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14th ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W. H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et al., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. Biologic entities include animals, plants, and the like. A biologic sample can contain whole cells and/or live cells and/or cell debris, and/or cell products, and/or virus particles. A biological sample can be or be from a product produced by a biologic entity, such as a bodily fluid, fruit, nut, pollen, and/or the like, whether still part of, connected to, associated with, or separated from the biologic entity that produced it. The biological sample can be obtained from an environment (e.g., water source, soil, air, and the like). Such samples are also referred to herein as environmental samples.

As used herein, the term “plant” includes reference to whole plants, plant organs (e.g., leaves, stems, roots, etc.), seeds and plant cells and progeny of same. Plant cell, as used herein includes, without limitation, seeds suspension cultures, embryos, meristematic regions, callus tissue, leaves, roots, shoots, gametophytes, sporophytes, pollen, and microspores. The class of plants which can be used in the methods of the invention is generally as broad as the class of higher plants amenable to transformation techniques, including both monocotyledonous and dicotyledonous plants.

As used herein, “engineered plant” includes reference to a plant that comprises within its genome a polynucleotide that has been modified to impart some functional change in the sequence, expression, gene product function, phenotype, and/or the like in the engineered plant. It will be appreciated that the engineered plant may be or may not be a “genetically modified organism” defined by the U.S. Department of Agriculture, Federal Drug Administration, or other appropriate regulatory authority and that any such classification does impact its standing as an engineered plant in the context of the present description. Generally, the modified polynucleotide is stably integrated within the genome such that the polynucleotide is passed on to successive generations. The modified polynucleotide may be integrated into the genome alone or as part of a recombinant expression cassette. “Engineered” is used herein to include any cell, cell line, callus, tissue, plant part or plant, the genotype of which has been altered by the presence of a modified nucleic acid including those engineered plants initially so altered as well as those created by sexual crosses or asexual propagation from the initial transgenic. The term “engineered” as used in this context herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

The terms “subject,” “individual,” are used interchangeably herein to refer to a plant, cell thereof, and/or progeny thereof.

As used herein, “control” can refer to an alternative subject or sample used in an experiment for comparison purpose and included to minimize or distinguish the effect of variables other than an independent variable.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “culturing” can refer to maintaining cells under conditions in which they can proliferate and avoid senescence as a group of cells. “Culturing” can also include conditions in which the cells also or alternatively differentiate.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “gene product” refers to a polynucleotide and/or polypeptide products produced from a transcribed and optionally translated gene. Gene products include products produced from post-transcriptional and post-translational modifications of the polynucleotide and/or polypeptide directly transcribed and optionally translated from the gene, including but not limited to splice variants and polypeptide products produced by cleavage of one or more portions of a pre- or prepro-protein.

As used herein, “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in embodiments modulation may encompass an increase in the value of the measured variable by about 10 to 500 percent or more. In embodiments, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In embodiments, modulation may encompass a decrease or reduction in the value of the measured variable by about 5 to about 100%. In some embodiments, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In embodiments, modulation may be specific or selective, hence, one or more desired phenotypic embodiments of a cell or cell population may be modulated without substantially altering other (unintended, undesired) phenotypic embodiment(s).

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used interchangeably herein, “operatively linked” and “operably linked” in the context of recombinant or engineered polynucleotide molecules (e.g. DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g., promoters, enhancers, and termination elements), and/or selectable markers in an expression vector. “Operatively linked” can also refer to an indirect attachment (i.e., not a direct fusion) of two or more polynucleotide sequences or polypeptides to each other via a linking molecule (also referred to herein as a linker).

As used herein, “overexpressed” or “overexpression” refers to an increased expression level of an RNA and/or protein product encoded by a gene as compared to the level of expression of the RNA or protein product in a normal or control cell. The amount of increased expression as compared to a normal or control cell can be about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.2, 2.4, 2.6, 2.8, 3.0, 3.3, 3.6, 3.9, 4.0, 4.4, 4.8, 5.0, 5.5, 6, 6.5, 7, 7.5, 8.0, 8.5, 9, 9.5, 10, 11, 12, 13, 14, 15, 16, 17, 18, 19, 20, 25, 30, 35, 40, 45, 50, 60, 70, 0, 90, 100 fold or more greater than the normal, unmodified, or control cell. “Increased expression” or “overexpression” are both used to refer to an increased expression of a gene, such as a transgene or gene product thereof in a sample as compared to the expression of said gene or gene product in a suitable control. The term “increased expression” refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control in some embodiments herein.

As used herein, “plasmid” refers to a non-chromosomal double-stranded DNA sequence including an intact “replicon” such that the plasmid is replicated in a host cell.

As used herein, “positive control” refers to a “control” that is designed to produce the desired result, provided that all reagents are functioning properly and that the experiment is properly conducted.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein “reduced expression” or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

As used herein, the term “specific binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “substantial” and “substantially,” specify an amount of between 95% and 100%, inclusive, between 96% and 100%, inclusive, between 97% and 100%, inclusive, between 98% and 100%, inclusive, or between 99% and 100%, inclusive.

As used herein, the term “vector” or is used in reference to a vehicle used to introduce an exogenous nucleic acid sequence into a cell. A vector may include a DNA molecule, linear or circular (e.g., plasmids), which includes a segment encoding an RNA and/or polypeptide of interest operatively linked to additional segments that provide for its transcription and optional translation upon introduction into a host cell or host cell organelles. Such additional segments can include promoter and/or terminator sequences, and can also include one or more origins of replication, one or more selectable markers, an enhancer, a polyadenylation signal, etc. Expression vectors are generally derived from yeast or bacterial genomic or plasmid DNA, or viral DNA, or may contain elements of both. Expression vectors can be adapted for expression in prokaryotic or eukaryotic cells. Expression vectors can be adapted for expression in mammalian, fungal, yeast, or plant cells. Expression vectors can be adapted for expression in a specific cell type via the specific regulator or other additional segments that can provide for replication and expression of the vector within a particular cell type.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Phosphorous is a crucial macronutrient necessary for plant growth and development and is a major limiting factor in crop yield in the developed world. Plants uptake phosphate in the form of inorganic phosphate (Pi), however, Pi is often limited in agricultural soils. In addition, Pi is a non-renewable resource with current reserves projected to be depleted in as early as 30 years (Cordell & White 2011; Herrera-Estrella & López-Arredondo 2016). As such, there is a need for plants, compositions, and methods for improved utilization of environmental phosphorous.

With that said and without being bound by theory, Applicant has at discovered and demonstrated herein that an increase in the synthesis of inositol pyrophosphates surprisingly drives an increase in the phosphorous starvation response and leveraging the increased synthesis can at least provide engineered plants that can be grown in conditions with sub-optimal phosphorus or phosphate availability. Certain example embodiments disclosed herein can provide engineered plants that have modified inositol pyrophosphates and/or synthesis thereof. In some embodiments, the engineered plants overexpress an inositol tetrakisphosphate kinase (ITPK) gene and/or gene product and/or a dual domain diphosphoinositol pentakisphosphate kinase (VIP) gene and/or gene product or a region thereof, such as the kinase domain of the VIP or the kinase domain the ITPK. In some embodiments, the engineered plants have reduced Pi accumulation and/or an increased phosphate starvation response. In some embodiments, the engineered plants have improved phosphorus-use efficiency. In some embodiments, the engineered plants can have improved survivability, productivity, growth, viability, and/or the like in an environment (e.g., soil) with low phosphorous (P) and/or inorganic phosphate (Pi) availability. In some embodiments, the engineered plants have a lower or eliminated requirement for supplemental P or Pi (e.g., a fertilizer). Also described in certain example embodiments herein are methods of growing the engineered plants that that overexpress an ITPK gene and/or gene product, a VIP gene and/or gene product, and/or the kinase domain an ITPK and/or VIP, particularly in an environment with low phosphorous/Pi availability.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered Plants

Described herein are engineered plant comprising increased inositol pyrophosphates as compared to a wild-type or unmodified plant.

In certain example embodiments, the plant comprises increased expression and/or amount of (a) an inositol tetrakisphosphate kinase (ITPK) gene and/or gene product (e.g., ITPK1 and/or ITPK2); (b) a diphosphoinositol pentakisphosphate (VIP) gene (e.g., VIP1 and/or VIP2) and/or gene product; or (c) both (a) and (b).

In certain example embodiments, the ITPK and VIP gene and/or gene product comprises or consists of or encodes a kinase domain of the ITPK gene product or VIP gene product. In certain example embodiments, the IPTK kinase domain is an ITPK1 kinase domain (ITPK1KD) or an ITPK2 kinase domain (ITPK2KD). In certain example embodiments, the VIP kinase domain is a VIP2 kinase domain (VIP2KD) or a VIP1 kinase domain (VIP1KD). In certain example embodiments, the increase in expression and/or amount of ITPK gene and/or gene product and/or VIP gene and/or gene product is 1-1,000 fold or more as compared to a suitable control, such as a wild-type or unmodified plant.

In certain example embodiments, the engineered plant has increased phosphorous use efficiency as compared to a wild-type or unmodified plant. In certain example embodiments, phosphorous use efficiency is increased 1-1,000 fold or more as compared to a suitable control, such as a wild-type or unmodified plant.

In certain example embodiments, the engineered plant has reduced Pi accumulation capability and/or capacity as compared to a wild-type or unmodified plant. In certain example embodiments, the Pi accumulation capability and/or capacity is reduced by 1-1,000 fold or more as compared to a suitable control, such as a wild-type or unmodified plant.

In certain example embodiments, the engineered plant has one or more modified developmental pathways and/or hormone signaling pathways as compared to a suitable control, such as a wild-type or unmodified plant.

In certain example embodiments, the engineered plant comprises one or more modifications to (a) an endogenous inositol ITPK gene and/or gene product; (b) an endogenous VIP gene and/or gene product; or (c) both (a) and (b)), wherein the one or more modifications increases the expression of an ITPK gene and/or gene product, a VIP gene and/or gene product, or both as compared to a suitable control, such as wild-type or unmodified plant or cell(s) thereof.

In certain example embodiments, the one or more modifications comprise an insertion of one or more nucleotides, a deletion of one or more nucleotides, a substitution of one or more nucleotides, or any combination thereof in the endogenous ITPK gene and/or gene product, endogenous VIP gene and/or gene product, or both.

In some embodiments, the engineered plant has about 0.01-100 fold or more increase in the expression of one or more ITPK and/or VIP genes or, increase in the amount of one or more ITPK and/or VIP gene products, and/or increase in the activity of one or more ITPK and/or VIP enzymes as compared to a suitable control. In some embodiments, the engineered plant has about 0.01-100 fold or more increase in the expression of one or more ITPK1, ITPK2, VIP1, VIP2 genes or, increase in the amount of one or more ITPK1, ITPK2, VIP1, VIP2 gene products, and/or increase in the activity of one or more ITPK1, ITPK2 VIP1, VIP2 enzymes as compared to a suitable control. In some embodiments, the engineered plant has about 0.01, to/or 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999 fold or more increase in the expression of one or more ITPK and/or VIP (e.g., ITPK1, ITPK2, VIP1, VIP2) genes, increase in the amount of one or more ITPK and/or VIP (e.g., ITPK1, ITPK2, VIP1, VIP2) gene products, and/or increase in the activity of one or more ITPK and/or VIP (e.g., ITPK1, VIP1, VIP2) gene products as compared to a suitable control.

In some embodiments, the engineered plant has about 1-1,000 percent or more increase in the expression of one or more ITPK and or VIP (e.g., ITPK1, ITPK2, VIP1, VIP2) genes, increase in the amount of one or more ITPK and/or VIP (e.g., ITPK1, ITPK2, VIP1, VIP2), gene products, and/or increase in the activity of one or more ITPK and/or VIP (e.g., ITPK1, ITPK2, VIP1, VIP2) gene products as compared to a suitable control. In some embodiments, the engineered plant has about a 1%, to/or 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, 501%, 502%, 503%, 504%, 505%, 506%, 507%, 508%, 509%, 510%, 511%, 512%, 513%, 514%, 515%, 516%, 517%, 518%, 519%, 520%, 521%, 522%, 523%, 524%, 525%, 526%, 527%, 528%, 529%, 530%, 531%, 532%, 533%, 534%, 535%, 536%, 537%, 538%, 539%, 540%, 541%, 542%, 543%, 544%, 545%, 546%, 547%, 548%, 549%, 550%, 551%, 552%, 553%, 554%, 555%, 556%, 557%, 558%, 559%, 560%, 561%, 562%, 563%, 564%, 565%, 566%, 567%, 568%, 569%, 570%, 571%, 572%, 573%, 574%, 575%, 576%, 577%, 578%, 579%, 580%, 581%, 582%, 583%, 584%, 585%, 586%, 587%, 588%, 589%, 590%, 591%, 592%, 593%, 594%, 595%, 596%, 597%, 598%, 599%, 600%, 601%, 602%, 603%, 604%, 605%, 606%, 607%, 608%, 609%, 610%, 611%, 612%, 613%, 614%, 615%, 616%, 617%, 618%, 619%, 620%, 621%, 622%, 623%, 624%, 625%, 626%, 627%, 628%, 629%, 630%, 631%, 632%, 633%, 634%, 635%, 636%, 637%, 638%, 639%, 640%, 641%, 642%, 643%, 644%, 645%, 646%, 647%, 648%, 649%, 650%, 651%, 652%, 653%, 654%, 655%, 656%, 657%, 658%, 659%, 660%, 661%, 662%, 663%, 664%, 665%, 666%, 667%, 668%, 669%, 670%, 671%, 672%, 673%, 674%, 675%, 676%, 677%, 678%, 679%, 680%, 681%, 682%, 683%, 684%, 685%, 686%, 687%, 688%, 689%, 690%, 691%, 692%, 693%, 694%, 695%, 696%, 697%, 698%, 699%, 700%, 701%, 702%, 703%, 704%, 705%, 706%, 707%, 708%, 709%, 710%, 711%, 712%, 713%, 714%, 715%, 716%, 717%, 718%, 719%, 720%, 721%, 722%, 723%, 724%, 725%, 726%, 727%, 728%, 729%, 730%, 731%, 732%, 733%, 734%, 735%, 736%, 737%, 738%, 739%, 740%, 741%, 742%, 743%, 744%, 745%, 746%, 747%, 748%, 749%, 750%, 751%, 752%, 753%, 754%, 755%, 756%, 757%, 758%, 759%, 760%, 761%, 762%, 763%, 764%, 765%, 766%, 767%, 768%, 769%, 770%, 771%, 772%, 773%, 774%, 775%, 776%, 777%, 778%, 779%, 780%, 781%, 782%, 783%, 784%, 785%, 786%, 787%, 788%, 789%, 790%, 791%, 792%, 793%, 794%, 795%, 796%, 797%, 798%, 799%, 800%, 801%, 802%, 803%, 804%, 805%, 806%, 807%, 808%, 809%, 810%, 811%, 812%, 813%, 814%, 815%, 816%, 817%, 818%, 819%, 820%, 821%, 822%, 823%, 824%, 825%, 826%, 827%, 828%, 829%, 830%, 831%, 832%, 833%, 834%, 835%, 836%, 837%, 838%, 839%, 840%, 841%, 842%, 843%, 844%, 845%, 846%, 847%, 848%, 849%, 850%, 851%, 852%, 853%, 854%, 855%, 856%, 857%, 858%, 859%, 860%, 861%, 862%, 863%, 864%, 865%, 866%, 867%, 868%, 869%, 870%, 871%, 872%, 873%, 874%, 875%, 876%, 877%, 878%, 879%, 880%, 881%, 882%, 883%, 884%, 885%, 886%, 887%, 888%, 889%, 890%, 891%, 892%, 893%, 894%, 895%, 896%, 897%, 898%, 899%, 900%, 901%, 902%, 903%, 904%, 905%, 906%, 907%, 908%, 909%, 910%, 911%, 912%, 913%, 914%, 915%, 916%, 917%, 918%, 919%, 920%, 921%, 922%, 923%, 924%, 925%, 926%, 927%, 928%, 929%, 930%, 931%, 932%, 933%, 934%, 935%, 936%, 937%, 938%, 939%, 940%, 941%, 942%, 943%, 944%, 945%, 946%, 947%, 948%, 949%, 950%, 951%, 952%, 953%, 954%, 955%, 956%, 957%, 958%, 959%, 960%, 961%, 962%, 963%, 964%, 965%, 966%, 967%, 968%, 969%, 970%, 971%, 972%, 973%, 974%, 975%, 976%, 977%, 978%, 979%, 980%, 981%, 982%, 983%, 984%, 985%, 986%, 987%, 988%, 989%, 990%, 991%, 992%, 993%, 994%, 995%, 996%, 997%, 998%, 999%, 1000% or more increase in the expression of one or more ITPK and/or VIP (e.g., ITPK1, ITPK2, VIP1, VIP2), genes, increase in the amount of one or more ITPK and/or VIP (e.g., ITPK1, ITPK2, VIP1, VIP2) gene products, and/or increase in the activity of one or more ITPK and/or VIP (e.g., ITPK1, ITPK2, VIP1, VIP2), enzymes as compared to a suitable control.

In some embodiments, the one or more ITPK and/or VIP (e.g., ITPK1, ITPK2, VIP1, VIP2) gene products are one or more ITPK and/or VIP (e.g., ITPK1, ITPK2, VIP1, VIP2) mRNAs, one or more ITPK and/or VIP (e.g., ITPK1, ITPK2, VIP1, VIP2) polypeptides, or both. In some embodiments the gene product is a VIP kinase domain. In some embodiments, the gene product is a VIP2 kinase domain or VIP1 kinase domain. In some embodiments, the gene product is an ITPK kinase domain. In some embodiments, the gene product is an ITPK1 or ITPK2 kinase domain.

In some embodiments, the engineered plant contains one or more modified ITPK and/or VIP (e.g., ITPK1, ITPK2, VIP1, VIP2) genes and/or gene products such that the engineered plant overexpresses one or more ITPK and/or VIP (e.g., ITPK1, ITPK2, VIP1, VIP2) polynucleotides and/or polypeptides as compared to a suitable control.

Without being bound by theory, the engineered plants described herein can have increased phosphorus use efficiency, increase in expression of, synthesis of, amount of, and/or activity of one or more inositol pyrophosphates. In some embodiments, the engineered plant has about 0.01-100 fold or more increase in (a) phosphorus use efficiency; (b) expression, synthesis, amount, and/or activity of one or more inositol pyrophosphates; or both (a) and (b) as compared to a suitable control. In some embodiments, the engineered plant has about 0.01, to/or 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999 fold or more increase in (a) phosphorus use efficiency; (b) expression, synthesis, amount, and/or activity of one or more inositol pyrophosphates; or both (a) and (b) as compared to a suitable control.

In some embodiments, the engineered plant has about 1-1,000 percent or more increase (a) phosphorus use efficiency; (b) expression, synthesis, amount, and/or activity of one or more inositol pyrophosphates; or both (a) and (b) as compared to a suitable control. In some embodiments, the engineered plant has about a 1%, to/or 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, 501%, 502%, 503%, 504%, 505%, 506%, 507%, 508%, 509%, 510%, 511%, 512%, 513%, 514%, 515%, 516%, 517%, 518%, 519%, 520%, 521%, 522%, 523%, 524%, 525%, 526%, 527%, 528%, 529%, 530%, 531%, 532%, 533%, 534%, 535%, 536%, 537%, 538%, 539%, 540%, 541%, 542%, 543%, 544%, 545%, 546%, 547%, 548%, 549%, 550%, 551%, 552%, 553%, 554%, 555%, 556%, 557%, 558%, 559%, 560%, 561%, 562%, 563%, 564%, 565%, 566%, 567%, 568%, 569%, 570%, 571%, 572%, 573%, 574%, 575%, 576%, 577%, 578%, 579%, 580%, 581%, 582%, 583%, 584%, 585%, 586%, 587%, 588%, 589%, 590%, 591%, 592%, 593%, 594%, 595%, 596%, 597%, 598%, 599%, 600%, 601%, 602%, 603%, 604%, 605%, 606%, 607%, 608%, 609%, 610%, 611%, 612%, 613%, 614%, 615%, 616%, 617%, 618%, 619%, 620%, 621%, 622%, 623%, 624%, 625%, 626%, 627%, 628%, 629%, 630%, 631%, 632%, 633%, 634%, 635%, 636%, 637%, 638%, 639%, 640%, 641%, 642%, 643%, 644%, 645%, 646%, 647%, 648%, 649%, 650%, 651%, 652%, 653%, 654%, 655%, 656%, 657%, 658%, 659%, 660%, 661%, 662%, 663%, 664%, 665%, 666%, 667%, 668%, 669%, 670%, 671%, 672%, 673%, 674%, 675%, 676%, 677%, 678%, 679%, 680%, 681%, 682%, 683%, 684%, 685%, 686%, 687%, 688%, 689%, 690%, 691%, 692%, 693%, 694%, 695%, 696%, 697%, 698%, 699%, 700%, 701%, 702%, 703%, 704%, 705%, 706%, 707%, 708%, 709%, 710%, 711%, 712%, 713%, 714%, 715%, 716%, 717%, 718%, 719%, 720%, 721%, 722%, 723%, 724%, 725%, 726%, 727%, 728%, 729%, 730%, 731%, 732%, 733%, 734%, 735%, 736%, 737%, 738%, 739%, 740%, 741%, 742%, 743%, 744%, 745%, 746%, 747%, 748%, 749%, 750%, 751%, 752%, 753%, 754%, 755%, 756%, 757%, 758%, 759%, 760%, 761%, 762%, 763%, 764%, 765%, 766%, 767%, 768%, 769%, 770%, 771%, 772%, 773%, 774%, 775%, 776%, 777%, 778%, 779%, 780%, 781%, 782%, 783%, 784%, 785%, 786%, 787%, 788%, 789%, 790%, 791%, 792%, 793%, 794%, 795%, 796%, 797%, 798%, 799%, 800%, 801%, 802%, 803%, 804%, 805%, 806%, 807%, 808%, 809%, 810%, 811%, 812%, 813%, 814%, 815%, 816%, 817%, 818%, 819%, 820%, 821%, 822%, 823%, 824%, 825%, 826%, 827%, 828%, 829%, 830%, 831%, 832%, 833%, 834%, 835%, 836%, 837%, 838%, 839%, 840%, 841%, 842%, 843%, 844%, 845%, 846%, 847%, 848%, 849%, 850%, 851%, 852%, 853%, 854%, 855%, 856%, 857%, 858%, 859%, 860%, 861%, 862%, 863%, 864%, 865%, 866%, 867%, 868%, 869%, 870%, 871%, 872%, 873%, 874%, 875%, 876%, 877%, 878%, 879%, 880%, 881%, 882%, 883%, 884%, 885%, 886%, 887%, 888%, 889%, 890%, 891%, 892%, 893%, 894%, 895%, 896%, 897%, 898%, 899%, 900%, 901%, 902%, 903%, 904%, 905%, 906%, 907%, 908%, 909%, 910%, 911%, 912%, 913%, 914%, 915%, 916%, 917%, 918%, 919%, 920%, 921%, 922%, 923%, 924%, 925%, 926%, 927%, 928%, 929%, 930%, 931%, 932%, 933%, 934%, 935%, 936%, 937%, 938%, 939%, 940%, 941%, 942%, 943%, 944%, 945%, 946%, 947%, 948%, 949%, 950%, 951%, 952%, 953%, 954%, 955%, 956%, 957%, 958%, 959%, 960%, 961%, 962%, 963%, 964%, 965%, 966%, 967%, 968%, 969%, 970%, 971%, 972%, 973%, 974%, 975%, 976%, 977%, 978%, 979%, 980%, 981%, 982%, 983%, 984%, 985%, 986%, 987%, 988%, 989%, 990%, 991%, 992%, 993%, 994%, 995%, 996%, 997%, 998%, 999%, 1000% or more increase (a) phosphorus use efficiency; (b) expression, synthesis, amount, and/or activity of one or more inositol pyrophosphates; or both (a) and (b) as compared to a suitable control.

In some embodiments, the ITPK and/or VIP (e.g., ITPK1, ITPK, 2, VIP1, VIP2) gene and/or gene product overexpressing plant accumulates less Pi than an unmodified plant or other suitable control. In some embodiments, the engineered plant accumulates about 0.01% to up to but excluding 100% less (with 100% being no Pi accumulation) Pi as compared to an unmodified plant or other suitable control. In some embodiments, the engineered plant accumulates about 0.01%, to/or 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, less Pi as compared as compared to an unmodified plant or other suitable control.

### ITPK Genes and Gene Products

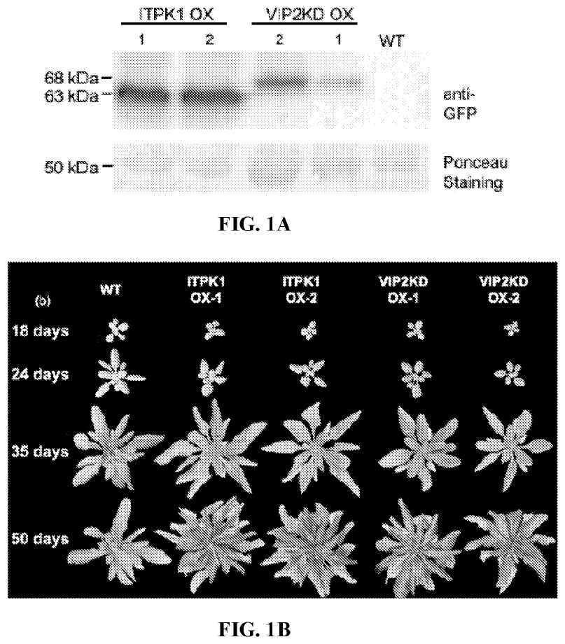

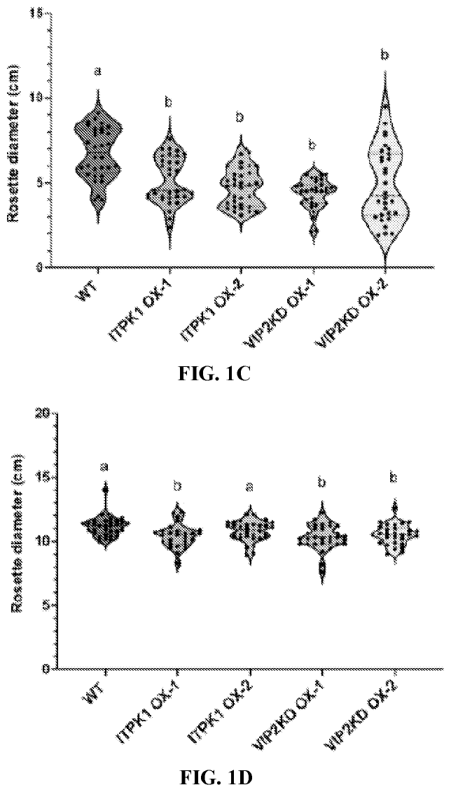

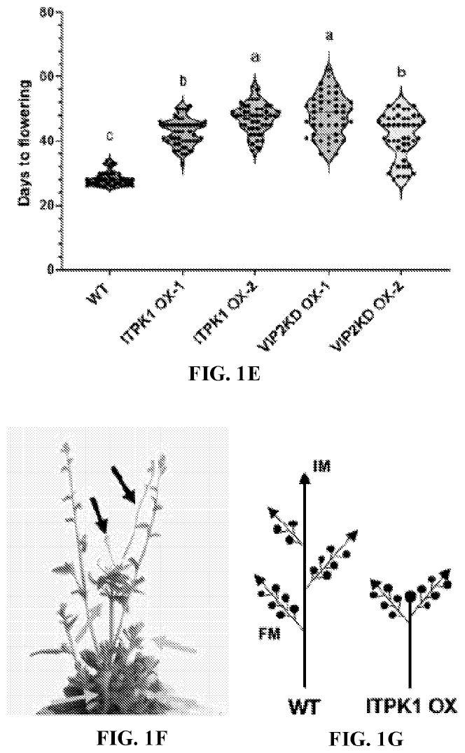

ITPK genes and gene products as the terms are used herein refers to an ITPK gene (e.g., am ITPK1 or ITPK2) and products produced therefrom by any native or synthetic synthesis, including but not limited to RNA (e.g., mRNA, and any other RNAs transcribed from an ITPK gene), polypeptides translated from an ITPK RNA, and any intermediates, splice variants, pro-proteins, pre-proproteins, glycosylation variants, and/or the like. ITPLK genes and//or gene products include ITPK1 (inositol tetrakisphosphate 1-kinase 1) and ITPK2 (inositol tetrakisphosphate kinase 2) genes and gene products (See e.g., Laha et al., (2019) ACS Chem Biol. 14:2127-2133). The term “an ITPK gene” as used herein refers to the gene encoding an ITPK (inositol tetrakisphosphate kinase) polypeptide or region(s) thereof capable of performing an ITPK enzymatic or other activity. The term “ITPK polypeptide” refers to a polypeptide that is the full length ITPK enzyme or is a functional fragment thereof that is capable of performing one or more enzymatic activities (including but not limited to the kinase domain of ITPK1 or ITPK2, which are described in e.g., Riemer et al., Front. Plant Sci. (2022) 13:944515, particularly at FIG. 1). In some embodiments, the ITPK gene product or functional fragment thereof is capable of phosphorylating InsP₆.

Tables 1-2 below provides reference sequences for exemplary ITPK genes and/or gene products. All homologues, orthologues, and structural and functional variants of an ITPK gene and/or gene product described herein are within the scope of the present description and will be appreciated by those of skill in the art in view of the description herein. Where a mutation is described or presented in the context of a reference ITPK sequence (DNA, RNA, or protein), one of ordinary skill in the art will, using generally available techniques and methodology known in the art, appreciate and understand corresponding mutations in variants, homologues, paralogues, and/or orthologues of the reference ITPK sequence.

TABLE 1
Reference ITPK Encoding Polynucleotides from Arabidopsis thaliana

| | mRNA (GenBank | NCBI Gene | | SEQ |
|---|---|---|---|---|
| ITPK | Accession No.) | ID | Sequence | ID NO: |
| ITPK1 | NM_121682 | 831539 | GTACAACATTCCCTTCTCTCTCTGTTATTTCATC | 1 |
| | | | TCTAGAAAAGGGCACAAAAAACCAAACTTTAC | |
| | | | GATTTCTCCTCTCTGGAATCTCGATTCTGTATG | |
| | | | ATCTCTTCGATCTAATCGAGTCTTCTAAACGTT | |
| | | | CCGTGTAGAATTTCAAATCTAGGGTTTGCTAA | |
| | | | AGCTTGAACCTTTCTTACCCTTTTCTCTCGAAG | |
| | | | AGGTTTTGATTCCTGGGCATGAATTTTTCCCTA | |
| | | | ATCTTGTTACCTTCGAAGCATAACAACAACGA | |
| | | | AAGATGTCAGATTCAATCCAGGAAAGATACTT | |
| | | | AGTTGGATACGCACTCGCAGCCAAGAAACAGC | |
| | | | ATAGTTTCATCCAACCTTCTTTGATCGAACACT | |
| | | | CAAGGCAACGAGGCATTGATCTGGTCAAGCTT | |
| | | | GATCCGACGAAATCGTTGTTGGAGCAAGGGAA | |
| | | | GCTTGATTGCATAATCCACAAGCTTTACGATGT | |
| | | | GTATTGGAAGGAGAATCTTCATGAGTTTCGCG | |
| | | | AGAAATGTCCTGGTGTTCCTGTAATCGATTTGC | |
| | | | CTGAGGCTATCGAGCGATTGCACAATAGGGTT | |
| | | | TCGATGCTTGAGGTGATAACTCAATTGAGGTTT | |
| | | | CCTGTTTCAGATAGCGAGAGATTTGGTGTCCC | |
| | | | GGAGCAAGTTGTTGTTATGGATTCGAGCGTCTT | |
| | | | GAGCGGTGGAGGAGCTTTAGGGGAGCTTAAGT | |
| | | | TTCCGGTGATTGCTAAGCCGTTGGATGCTGATG | |
| | | | GGAGTGCTAAATCTCATAAGATGTTTTTGATTT | |
| | | | ATGATCAAGAAGGGATGAAGATTTTGAAAGCT | |
| | | | CCTATTGTGTTGCAGGAGTTTGTGAATCATGGT | |
| | | | GGTGTGATCTTTAAGGTCTATGTGGTTGGAGAT | |
| | | | CATGTCAAATGTGTAAAGAGAAGATCTTTACC | |
| | | | TGATATCTCTGAAGAGAAGATTGGGACGTCGA | |
| | | | AAGGGTCTCTTCCGTTTTCGCAGATATCGAATC | |
| | | | TGACTGCTCAAGAGGACAAGAACATTGAGTAT | |
| | | | GGTGAGGATAGGAGCTTAGAGAAAGTGGAAA | |
| | | | TGCCTCCGTTAAGTTTCTTGACAGATCTAGCAA | |
| | | | AGGCCATGAGGGAATCAATGGGACTCAATCTC | |
| | | | TTTAACTTCGACGTGATTAGGGATGCCAAAGA | |
| | | | TGCTAATAGGTACCTTATAATTGATATTAACTA | |
| | | | CTTTCCTGGATATGCTAAGATGCCCTCTTACGA | |
| | | | GCCTGTGTTGACTGAGTTCTTCTGGGACATGGT | |
| | | | CACTAAGAAGAATCATGTCTGAGAAATAATCT | |
| | | | GTTGGGGAGTTAGCAGGAACATTGTTGGGGAG | |
| | | | GATTTGTACCATTGCAATGTGTTGCAACTGCCA | |
| | | | TTGCAATTCTGGCCTATGGTGACTAGCTGCTTT | |
| | | | GCTAATTTTCATTTGGATTCTTGAACTTGTGCT | |
| | | | TTTTATATTTTTTTAGTTGTTTCTCTTTTGTTTA | |
| | | | CTTCCGAGTTTTGCAAAGCCATGTTCAAATGCA | |
| | | | TAACTGTTGCTACTCTTGAAAAGCATTGAATCT | |
| | | | CTAAATTCTTGGCAATGTGGTCTGTTCTCAACT | |
| | | | GTTGGTAGTTTGTGATTTCTTTTCAAGCTTTTAT | |
| | | | GGTGCCTGTTTTCTTCCTCAAATCTGACAAGAC | |
| | | | TGAGATATTTGATTAAGCATTGGTATAAGTTG | |
| | | | ATTAGTAACCTTCACTTGGCAATGCTTTTCCAT | |
| | | | TCTCTATATTATGAACTGTTTATGTGAAACCGG | |
| | | | TGCTAAGTCTTTGTATCGGATTTCTAAGACATG | |
| | | | G | |
| ITPK2 | NM_001085021 | 829519 | CCCAAGCTTGAACTTCTTGCTAGGTAACCAAT | 2 |
| | | | AACAAACCCAAAATGCTTCTTTGGAGTTATAA | |
| | | | AGTTTTGATTTTTCATTGCAAAGTTTCCAAATT | |
| | | | TGTTGAACAATCTTTTTTGGTTCTGCTTTTTCTT | |
| | | | TCCTGAGAGACGACTGTTCCTTTGTTGGCGTTT | |
| | | | AAACATAACCGACAAACTCTTTTCTACTTTCAA | |
| | | | AAAGATCTTTAGGACTTTGACATTTTCTTCCAG | |
| | | | ATTTGGTTCTTGTTTCCTTACTCTTGTTTTGGTA | |
| | | | CCTATTATTGGTTTGCCTGATTCTATGAGGACC | |
| | | | ATTGCCACTTGCCATGTCTTGTCACCTGCCATG | |
| | | | TTTTTGTCCTTAATCGGCTCTTTTGCTTAGGGTT | |
| | | | AGGCTTACACTAGTATACTCTACTTGTGGTATC | |
| | | | AGGATGTTTCTATGGGAATATTCCTTGTGTAAT | |
| | | | CCTTTTGGATTAGTCTCTATTGAGGTTTGACTC | |
| | | | TGGCTTTTAAAAAATAAAATACTTAGATGTTTT | |
| | | | GCCTTTTCTGTGGCTTGGAAATCTTCTTCAACC | |
| | | | ACATGGGTGTTTTTGTTAAAGCAATCACTGGTT | |
| | | | CTACTTAATATGTCATCTTCTAGAATGTCTCTA | |
| | | | ATAGGTCAGAGAGAACATTTCTTATTGGTTTA | |
| | | | GTTTGACGTCTGGTCTCTGTTTTGCTTCTTTCTA | |
| | | | ATCAACCCATAGCTTTCATTCTTTTTCTTCTTTC | |
| | | | AGCAGTGTTTTCAAGAAAATGCTAGTAATACT | |
| | | | TTTTGTGTCTGTCAATTTCAGGAGAAAAGGCAT | |
| | | | TTTCTTTGTCGCCATCGATTTGAACCGGCCCCT | |
| | | | ATCTGAACAAGGGCCATTTGATGTTGTTTTGCA | |
| | | | TAAGTTGTTGGGAAAAGAGTGGGAAGAGGTTA | |
| | | | TTGAGGATTACCAACAAAAACACCCAGAAGTG | |
| | | | ACTGTGCTTGATCCTCCAGGATCAATACAGCG | |
| | | | TATATATAATCGACAATCGATGCTTCAGGGTA | |
| | | | TGGCAGATTTGAAACTGTCAGATTGCAGTGGC | |
| | | | AGCCTTTTTGTTCCAAAGCAAATGGTTGTCTTG | |
| | | | AAAGATTCAGCAGCTAGTGCTGATGCAGTTGT | |
| | | | GGAAGCTGGTCTCAAATTTCCACTAGTTGCAA | |
| | | | AGCCGCTCTGGATCGATGGGACTGCAAAGTCA | |
| | | | CATCAATTGTACTTGGCTTATGACAGGCGCTCG | |
| | | | CTTGCAGAGCTTGATCCGCCTTTAGTCCTTCAA | |
| | | | GAGTTTGTTAATCATGGTGGAGTTATGTTCAAG | |
| | | | GTATTTGTGGTGGGTGATGTTATAAAAGTCAT | |
| | | | GAGACGGTTTTCTCTACCAAATGTGAGTAATT | |
| | | | GTGAAAAAGCCAAAGTTGATGGCGTCTTCCAA | |
| | | | TTCCCAAGGGTTTCATCAGCTGCTGCTTCAGCT | |
| | | | GATAACGCAGACTTGGACCCTCGTGTTGCTGA | |
| | | | GCTACCTCCAAAGCCTTTCCTCGAGGCGCTTGT | |
| | | | GAAAGAGCTAAGAAGCTTATTGGGACTTCGGC | |
| | | | TTTTCAACATAGACATGATCAGGGAACATGGG | |
| | | | AGCAAAAACGTGTTTTATGTTATTGACATCAA | |
| | | | CTATTTTCCTGGTTACGGAAAACTGCCAGACTA | |
| | | | CGAGCAAGTCTTTGTAGATTTCTTCCAAAATCT | |
| | | | GGCGCAGGTCAAATATAAGAAGAGACAACATT | |
| | | | GTAAATGAAAGAAAATGGCGGCAGTTTTTAGA | |
| | | | TGGTCTACTAAGAAGCGACAAATAATAAAATG | |
| | | | TCTAATTATGGATTTGTACAGAATTTAGCTCTC | |
| | | | CCTTTTGGAAGCAGTATCAGATAAACAAGTTT | |
| | | | TGATTACTGATTTTGAATTTTCAGTGAAATAAA | |
| | | | CGTCTCTTCATCCAAAGTTGAC | |
| ITPK2 | BT029235.1 | At4g33770 | ATGTTTGGGACTCTTGCTTCCGGCGAAATCGA | 9 |
| | | | AACTGCCAGACTGAACCGTAATTTGGGGATTA | |
| | | | CGTCCAATTTAGGGGTTTCCTGCGGTGGATTTG | |
| | | | AAGACTTTGCTATGAGATTCGAAGGGGAAAAC | |
| | | | ATGGTGCCATACAAAGGTGAAGAGCAGGAGG | |
| | | | AGGAAGAAGACCAAGTGGTGGTGAACGAAAC | |
| | | | GACGCCGTTCCAGTTTCAACAACCTTTGTTTCT | |
| | | | GCAGCAGCAGCAGAAGCTCGTTGTTGGGTATG | |
| | | | CTCTGACTTCTAAGAAGAAGAAGAGTTTCTTG | |
| | | | CAGCCCAAGCTTGAACTTCTTGCTAGGAGAAA | |
| | | | AGGCATTTTCTTTGTCGCCATCGATTTGAACCG | |
| | | | GCCCCTATCTGAACAAGGGCCATTTGATGTTGT | |
| | | | TTTGCATAAGTTGTTGGGAAAAGAGTGGGAAG | |
| | | | AGGTTATTGAGGATTACCAACAAAAACACCCA | |
| | | | GAAGTGACTGTGCTTGATCCTCCAGGATCAAT | |
| | | | ACAGCGTATATATAATCGACAATCGATGCTTC | |
| | | | AGGGTATGGCAGATTTGAAACTGTCAGATTGC | |
| | | | AGTGGCAGCCTTTTTGTTCCAAAGCAAATGGTT | |
| | | | GTCTTGAAAGATTCAGCAGCTAGTGCTGATGC | |
| | | | AGTTGTGGAAGCTGGTCTCAAATTTCCACTAGT | |
| | | | TGCAAAGCCGCTCTGGATCGATGGGACTGCAA | |
| | | | AGTCACATCAATTGTACTTGGCTTATGACAGG | |
| | | | CGCTCGCTTGCAGAGCTTGATCCGCCTTTAGTC | |
| | | | CTTCAAGAGTTTGTTAATCATGGTGGAGTTATG | |
| | | | TTCAAGGTATTTGTGGTGGGTGATGTTATAAA | |
| | | | AGTCATGAGACGGTTTTCTCTACCAAATGTGA | |
| | | | GTAATTGTGAAAAAGCCAAAGTTGATGGCGTC | |
| | | | TTCCAATTCCCAAGGGTTTCATCAGCTGCTGCT | |
| | | | TCAGCTGATAACGCAGACTTGGACCCTCGTGT | |
| | | | TGCTGAGCTACCTCCAAAGCCTTTCCTCGAGGC | |
| | | | GCTTGTGAAAGAGCTAAGAAGCTTATTGGGAC | |
| | | | TTCGGCTTTTCAACATAGACATGATCAGGGAA | |
| | | | CATGGGAGCAAAAACGTGTTTTATGTTATTGA | |
| | | | CATCAACTATTTTCCTGGTTACGGAAAACTGCC | |
| | | | AGACTACGAGCAAGTCTTTGTAGATTTCTTCCA | |
| | | | AAATCTGGCGCAGGTCAAATATAAGAAGAGAC | |
| | | | AACATTGTAAATGA | |
| ITPK2 | NM_119535.5 | AT4G33770 | TTTCAAGTTGCTTGTAATATGGTTTCATCTTGA | 10 |
| | | | GAAAAAAAAAAACAGAAAGAGAAAATACAAT | |
| | | | AAATGTATTCAGAAAAGAAGAATAACCAAAA | |
| | | | AAAGCAGCAAATCTTTGGGAACGACCATGTCA | |
| | | | CTGTAGCATCTGTTTTTCTGCAAATTATTCTCT | |
| | | | CTATAAATATATTTGATCATCAGACCAATCTCT | |
| | | | CCCTCAACATTTCCTCATCTCATCTCTCTGATTT | |
| | | | CTTGCTTCGAATTTTGATTTATCTCTCTCTGTCT | |
| | | | CTCTCTCGGTCTCGTGCTCTATCTCGTTTGTAG | |
| | | | GCAGTGCCAATTTCACCACTTGTGTTGTGTGTC | |
| | | | AGCGGATATAGAATCTGAGAGGAATATTTAAA | |
| | | | GATTCTCCCTTTGCTTCTTCTTTTTTATACCGTT | |
| | | | TCTATTAATGTTTGGGACTCTTGCTTCCGGCGA | |
| | | | AATCGAAACTGCCAGACTGAACCGTAATTTGG | |
| | | | GGATTACGTCCAATTTAGGGGTTTCCTGCGGTG | |
| | | | GATTTGAAGACTTTGCTATGAGATTCGAAGGG | |
| | | | GAAAACATGGTGCCATACAAAGGTGAAGAGC | |
| | | | AGGAGGAGGAAGAAGACCAAGTGGTGGTGAA | |
| | | | CGAAACGACGCCGTTCCAGTTTCAACAACCTT | |
| | | | TGTTTCTGCAGCAGCAGCAGAAGCTCGTTGTT | |
| | | | GGGTATGCTCTGACTTCTAAGAAGAAGAAGAG | |
| | | | TTTCTTGCAGCCCAAGCTTGAACTTCTTGCTAG | |
| | | | GAGAAAAGGCATTTTCTTTGTCGCCATCGATTT | |
| | | | GAACCGGCCCCTATCTGAACAAGGGCCATTTG | |
| | | | ATGTTGTTTTGCATAAGTTGTTGGGAAAAGAG | |
| | | | TGGGAAGAGGTTATTGAGGATTACCAACAAAA | |
| | | | ACACCCAGAAGTGACTGTGCTTGATCCTCCAG | |
| | | | GATCAATACAGCGTATATATAATCGACAATCG | |
| | | | ATGCTTCAGGGTATGGCAGATTTGAAACTGTC | |
| | | | AGATTGCAGTGGCAGCCTTTTTGTTCCAAAGC | |
| | | | AAATGGTTGTCTTGAAAGATTCAGCAGCTAGT | |
| | | | GCTGATGCAGTTGTGGAAGCTGGTCTCAAATT | |
| | | | TCCACTAGTTGCAAAGCCGCTCTGGATCGATG | |
| | | | GGACTGCAAAGTCACATCAATTGTACTTGGCT | |
| | | | TATGACAGGCGCTCGCTTGCAGAGCTTGATCC | |
| | | | GCCTTTAGTCCTTCAAGAGTTTGTTAATCATGG | |
| | | | TGGAGTTATGTTCAAGGTATTTGTGGTGGGTG | |
| | | | ATGTTATAAAAGTCATGAGACGGTTTTCTCTAC | |
| | | | CAAATGTGAGTAATTGTGAAAAAGCCAAAGTT | |
| | | | GATGGCGTCTTCCAATTCCCAAGGGTTTCATCA | |
| | | | GCTGCTGCTTCAGCTGATAACGCAGACTTGGA | |
| | | | CCCTCGTGTTGCTGAGCTACCTCCAAAGCCTTT | |
| | | | CCTCGAGGCGCTTGTGAAAGAGCTAAGAAGCT | |
| | | | TATTGGGACTTCGGCTTTTCAACATAGACATGA | |
| | | | TCAGGGAACATGGGAGCAAAAACGTGTTTTAT | |
| | | | GTTATTGACATCAACTATTTTCCTGGTTACGGA | |
| | | | AAACTGCCAGACTACGAGCAAGTCTTTGTAGA | |
| | | | TTTCTTCCAAAATCTGGCGCAGGTCAAATATA | |
| | | | AGAAGAGACAACATTGTAAATGAAAGAAAAT | |
| | | | GGCGGCAGTTTTTAGATGGTCTACTAAGAAGC | |
| | | | GACAAATAATAAAATGTCTAATTATGGATTTG | |
| | | | TACAGAATTTAGCTCTCCCTTTTGGAAGCAGTA | |
| | | | TCAGATAAACAAGTTTTGATTACTGATTTTGAA | |
| | | | TTTTCAGTGAAATAAACGTCTCTTCATCCAAAG | |
| | | | TTGACCAAACGATATCTGAATTCTTATTTTGAC | |
| | | | CATAGCGGTTCG | |

TABLE 2
Reference ITPK proteins from Arabidopsis thaliana

| | Protein (GenBank | | SEQ |
|---|---|---|---|
| ITPK | Accession No.) | Reference Sequence | ID NO: |
| ITPK1 | NP_197178 | MSDSIQERYLVGYALAAKKQHSFIQPSLIEHSRQRGIDLVKL | 3 |
| | | DPTKSLLEQGKLDCIIHKLYDVYWKENLHEFREKCPGVPVID | |
| | | LPEAIERLHNRVSMLEVITQLRFPVSDSERFGVPEQVVVMDS | |
| | | SVLSGGGALGELKFPVIAKPLDADGSAKSHKMFLIYDQEGM | |
| | | KILKAPIVLQEFVNHGGVIFKVYVVGDHVKCVKRRSLPDISE | |
| | | EKIGTSKGSLPFSQISNLTAQEDKNIEYGEDRSLEKVEMPPLS | |
| | | FLTDLAKAMRESMGLNLFNFDVIRDAKDANRYLIIDINYFPG | |
| | | YAKMPSYEPVLTEFFWDMVTKKNHV | |
| ITPK2 | NP_195103 | MFGTLASGEIETARLNRNLGITSNLGVSCGGFEDFAMRFEGE | 4 |
| | | NMVPYKGEEQEEEEDQVVVNETTPFQFQQPLFLQQQQKLV | |
| | | VGYALTSKKKKSFLQPKLELLARRKGIFFVAIDLNRPLSEQG | |
| | | PFDVVLHKLLGKEWEEVIEDYQQKHPEVTVLDPPGSIQRIYN | |
| | | RQSMLQGMADLKLSDCSGSLFVPKQMVVLKDSAASADAV | |
| | | VEAGLKFPLVAKPLWIDGTAKSHQLYLAYDRRSLAELDPPL | |
| | | VLQEFVNHGGVMFKVFVVGDVIKVMRRFSLPNVSNCEKAK | |
| | | VDGVFQFPRVSSAAASADNADLDPRVAELPPKPFLEALVKE | |
| | | LRSLLGLRLFNIDMIREHGSKNVFYVIDINYFPGYGKLPDYE | |
| | | QVFVDFFQNLAQVKYKKRQHCK | |
| ITPK2 | NP_001078490.1 | MLVILFVSVNFRRKGIFFVAIDLNRPLSEQGPFDVVLHKLLG | 11 |
| | | KEWEEVIEDYQQKHPEVTVLDPPGSIQRIYNRQSMLQGMAD | |
| | | LKLSDCSGSLFVPKQMVVLKDSAASADAVVEAGLKFPLVA | |
| | | KPLWIDGTAKSHQLYLAYDRRSLAELDPPLVLQEFVNHGGV | |
| | | MFKVFVVGDVIKVMRRFSLPNVSNCEKAKVDGVFQFPRVSS | |
| | | AAASADNADLDPRVAELPPKPFLEALVKELRSLLGLRLFNID | |
| | | MIREHGSKNVFYVIDINYFPGYGKLPDYEQVFVDFFQNLAQ | |
| | | VKYKKRQHCK | |
| ITPK2 | ANM66836.1 | MLQGMADLKLSDCSGSLFVPKQMVVLKDSAASADAVVEA | 12 |
| | | GLKFPLVAKPLWIDGTAKSHQLYLAYDRRSLAELDPPLVLQ | |
| | | EFVNHGGVMFKVFVVGDVIKVMRRFSLPNVSNCEKAKVDG | |
| | | VFQFPRVSSAAASADNADLDPRVAELPPKPFLEALVKELRSL | |
| | | LGLRLFNIDMIREHGSKNVFYVIDINYFPGYGKLPDYEQVFV | |
| | | DFFQNLAQVKYKKRQHCK | |

In some embodiments, the ITPK gene or gene product contains or is composed completely of a polynucleotide. In some embodiments, the ITPK gene or gene product contains or is completely composed of a sequence that is about 70% to 100% identical to any one of SEQ ID NO: 1-2 or 9-10 and/or a sequence complementary thereto. In some embodiments, the ITPK gene or gene product contains or is completely composed of a sequence that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 1-2 or 9-10 and/or a sequence complementary thereto.

In some embodiments, the ITPK gene product contains or is composed completely of a polypeptide. In some embodiments, the ITPK gene product contains or is completely composed of a sequence that is about 70% to 100% identical to any one of SEQ ID NO: 3-4 or 11-12. In some embodiments, the ITPK gene product contains or is completely composed of a sequence that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 3-4 or 11-12. In some embodiments, the gene encodes only the kinase domain of an ITPK gene. In some embodiments, the ITPK gene product is an ITPK kinase domain. In some embodiments, the ITPK kinase domain comprises or is composed only of amino acids 99-318 of SEQ ID NO: 3 or amino acids corresponding thereto in a variant, homologue, orthologue, or paralogue thereof. In some embodiments, the ITPK kinase domain comprises or is composed entirely of a polypeptide that is about 70% to 100% identical to amino acids 99-318 of SEQ ID NO: 3 or amino acids corresponding thereto in a variant, homologue, orthologue, or paralogue thereof. In some embodiments, the ITPK kinase domain comprises or is composed entirely of a polypeptide that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to amino acids 99-318 of SEQ ID NO: 3 or amino acids corresponding thereto in a variant, homologue, orthologue, or paralogue thereof. In some embodiments, the ITPK gene product is an ITPK kinase domain. In some embodiments, the ITPK kinase domain comprises or is composed only of amino acids 178-384 of SEQ ID NO: 4 or amino acids corresponding thereto in a variant, homologue, orthologue, or paralogue thereof. In some embodiments, the ITPK kinase domain comprises or is composed entirely of a polypeptide that is about 70% to 100% identical to amino acids 178-384 of SEQ ID NO: 4 or amino acids corresponding thereto in a variant, homologue, orthologue, or paralogue thereof. In some embodiments, the ITPK kinase domain comprises or is composed entirely of a polypeptide that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to amino acids 178-384 of SEQ ID NO: 4 or amino acids corresponding thereto in a variant, homologue, orthologue, or paralogue thereof.

### VIP Genes and Gene Products

VIP genes and gene products as the terms are used herein refers to a VIP gene (e.g., a VIP1 or VIP2 gene or portion thereof) and products produced therefrom by any native or synthetic synthesis, including but not limited to RNA (e.g., mRNA, and any other RNAs transcribed from a VIP gene), polypeptides translated from a VIP RNA, and any intermediates, splice variants, pro-proteins, pre-proproteins, glycosylation variants, and/or the like. VIP genes and//or gene products include VIP1 and VIP2 genes and gene products (See e.g., Zhu et al., eLIFE, 2019. 8: e43582). The term “a VIP gene” as used herein refers to the gene encoding a VIP polypeptide or region(s) thereof capable of performing a VIP enzymatic or other activity. The term “VIP polypeptide” refers to a polypeptide that is the full length VIP enzyme or is a functional fragment thereof that is capable of performing one or more enzymatic activities (including but not limited to the kinase domain of VIP1 or VIP2, which are described in e.g., Riemer et al., Front. Plant Sci. (2022) 13:944515, particularly at FIG. 1. In some embodiments, the VIP gene product or functional fragment thereof is capable of phosphorylating InsP₆.

Tables 3-4 below provides reference sequences for exemplary VIP genes and/or gene products. All homologues, orthologues, and structural and functional variants of a VIP gene and/or gene product described herein are within the scope of the present description and will be appreciated by those of skill in the art in view of the description herein. Where a mutation is described or presented in the context of a reference VIP sequence (DNA, RNA, or protein), one of ordinary skill in the art will, using generally available techniques and methodology known in the art, appreciate and understand corresponding mutations in variants, homologues, paralogues, and/or orthologues of the reference VIP sequence.

TABLE 3
Reference VIP Encoding Polynucleotides from Arabidopsis thaliana

| | mRNA (GenBank | NCBI Gene | | SEQ |
|---|---|---|---|---|
| VIP | Accession No.) | ID | Sequence | ID NO: |
| VIP2 | NM_121511 | 831359 | AATTCTGATTCAAAGCAACGCCTCTGACGCCT | 5 |
| (VIH1) | | | GAATCTTCTGGCTCTCTCTCTTTCCTCGTGCCA | |
| | | | TCAGCGCACTATCTCTAGCTATTGCTCTCTCTG | |
| | | | CTTGTGATCATCATTTTCCCCAACGGAGAACG | |
| | | | AGAGAACGTCGTTCGAGGAACGAAACGGAAA | |
| | | | GAGGTTCGTATCGGGAGGAGAGAGCGAAAAT | |
| | | | GGGGGTGGAAGAAGGAGCTGGTGTTGATAAG | |
| | | | AAGATAACGATTGGAGTCTGCGTCATGGAAAA | |
| | | | GAAGGTTTTTTCAGCTCCCATGGGACAAATTAT | |
| | | | GGACAGGATACATGCGTTTGGCGAATTTGAGA | |
| | | | TTATACATTTTGGGGACAAGGTCATACTTG | |
| | | | AAGACCCAGTAGAAAGTTGGCCGATTTGTGAT | |
| | | | TGTTTGATTGCTTTCTATTCCTCTGGATATCCTC | |
| | | | TTGAGAAAGTTCAAGCATATTCTTCTTTAAGAA | |
| | | | AGCCCTTTTTAGTGAATGAACTCGATCCCCAAT | |
| | | | ATCTTCTTCATGACCGCCGGAAAGTGTATGAG | |
| | | | CATCTTGAGATGTATGGTATCCCAGTTCCTAGA | |
| | | | TATGCTTGTGTCAATAGAAAAGTACCAGACGA | |
| | | | AGATCTTGATTATTTCGTCGAGGAAGAAGATT | |
| | | | TTGTTGAGGTTAAAGGTGAACGATTCTGGAAG | |
| | | | CCATTTGTGGAAAAGCCCGTCAATGGAGATGA | |
| | | | CCATAGTATAATGATATACTACCCTAGCTCAG | |
| | | | CTGGTGGAGGCATGAAAGAATTGTTTCGTAAG | |
| | | | GTTGGGAATCGATCAAGTGAGTTTCATCCTGA | |
| | | | TGTCAGAAGAGTAAGGAGAGAAGGTTCTTATA | |
| | | | TATATGAGGAGTTTATGCCTACTGGGGGAACT | |
| | | | GATGTCAAGGTCTACACTGTGGGTCCCGAATA | |
| | | | TGCACACGCTGAAGCAAGAAAATCACCTGTTG | |
| | | | TTGATGGTGTAGTAATGAGAAATCCAGATGGG | |
| | | | AAGGAAGTGAGATATCCAGTTTTACTTACACC | |
| | | | TGCTGAGAAACAAATGGCGAGAGAAGTTTGCA | |
| | | | TTGCATTTAGGCAAGCGGTTTGCGGTTTTGATC | |
| | | | TCCTACGATCTGAGGGAAGTTCATATGTTTGTG | |
| | | | ACGTAAATGGATGGAGTTTTGTGAAGAACTCC | |
| | | | TATAAGTATTACGACGATGCTGCTTGTGTGCTA | |
| | | | CGGAAAATGTTTTTGGATGCAAAGGCTCCTCA | |
| | | | TCTTTCTTCAACAATTCCTCCCATTCTGCCATG | |
| | | | GAAAATCAACGAACCTGTCCAATCTAATGAAG | |
| | | | GTCTTACCCGCCAAGGAAGTGGCATCATCGGA | |
| | | | ACTTTTGGGCAGTCAGAGGAGCTACGCTGTGT | |
| | | | CATTGCTATTGTTCGGCATGGTGATAGAACCCC | |
| | | | CAAGCAGAAAGTGAAACTAAAAGTTACAGAG | |
| | | | GAGAAACTGTTAAACCTGATGTTGAAGTACAA | |
| | | | TGGAGGAAAACCAAGAGCTGAGACAAAACTT | |
| | | | AAAACTGCCGTCCAATTGCAAGATCTATTGGA | |
| | | | TGCCACAAGAATGTTAATTCCTCGTGCAAGAT | |
| | | | CAGGTGAAAGTGATAGTGATGCAGAAGACCTT | |
| | | | GAACACGCTGACAAGCTTCGGCAAGTTAAAGC | |
| | | | CGTTCTTGAAGAGGGTGGGCATTTCTCTGGCAT | |
| | | | ATACAGGAAGGTTCAACTGAAGCCGTTGAAAT | |
| | | | GGGTTAACGTACCAAAAAGTGATGGTGAAGGC | |
| | | | GAAGAGGAACGCCCAGTGGAAGCTCTTATGGT | |
| | | | TCTGAAATACGGGGGTGTTCTAACTCATGCTG | |
| | | | GTAGAAAGCAGGCCGAAGAGCTTGGTAGATAC | |
| | | | TTTCGAAACAATATGTATCCAGGGGAAGGTAC | |
| | | | CGGTTTGCTCCGTCTCCATAGTACATACCGTCA | |
| | | | CGACCTTAAAATTTACAGCTCTGACGAGGGAC | |
| | | | GTGTTCAGATGTCTGCAGCTGCTTTTGCCAAAG | |
| | | | GCCTGCTTGACCTAGAAGGACAGCTGACCCCA | |
| | | | ATCCTGGTTTCTCTGGTAAGTAAGGACTCTTCC | |
| | | | ATGTTGGATGGCCTTGATAATGCCAGCAGTGA | |
| | | | AATGGAAGCAGCTAAGGCTCAACTGAATGAGA | |
| | | | TCATAACCGCCGGCTCAAAGATGGTACATGAT | |
| | | | CACGTCTCTTCTGAATTACCTTGGATGACTGAT | |
| | | | GGGGCTGGACTTCCTCCTCACGCTGATGAGCA | |
| | | | CCTACCTGAATTGGTAAAATTAGCTAAAAAGG | |
| | | | TGACTGAACAAGTAAGGCTACTTGCACAAGAT | |
| | | | GAACACGAGAATCTCGCTGAGCCTAGCGCTTA | |
| | | | TGATGTAGTCCCTCCATATGATCAAGCCAAGG | |
| | | | CCCTTGGCAAGTCAAACATTGACGTTGGTCGG | |
| | | | ATTGCTGCCGGATTACCTTGTGGTAGTGAAGG | |
| | | | GTTCCTTTTGATGTTTGCCCGGTGGAGAAAACT | |
| | | | CGAAAGGGATCTCTACAACGAAAGAAGAGAG | |
| | | | CGGTTTGACATTACGCAGATTCCTGATGTTTAC | |
| | | | GATTCATGCAAGTATGACCTGTTACATAATTCT | |
| | | | CATCTCGATCTAAAAGGACTAGACGAACTCTT | |
| | | | CAAAGTAGCGCAGTTGCTTGCAGATGGTGTAA | |
| | | | TCCCAAATGAGTATGGGATCAATCCACAGCAA | |
| | | | AAGCTCAAAATCGGTTCAAAGATTGCACGGCG | |
| | | | CCTGCTGGGTAAAATCCTTATTGACTTGAGGA | |
| | | | ACACTCGAGAAGAGGCCATGAGTGTTGCTGAA | |
| | | | TTGAAGAACAGTCAAGATCAAGTCTCAGTGTC | |
| | | | ATTGTATTCGTCAAGAAAGGAGGATAGATATA | |
| | | | GTCAACCAAAACTTTTCGTTAAAAGCGATGAG | |
| | | | TTGAGACGGCCTAGCACTGGAGAGAATAAAGA | |
| | | | AGAAGATGATGATAAAGAAACCAAATACAGA | |
| | | | CTAGATCCAAAGTATGCAAATGTCATGACGCC | |
| | | | TGAACGTCATGTGAGGACACGTCTTTACTTCAC | |
| | | | ATCTGAATCACATATTCACTCTCTGATGAACGT | |
| | | | TCTACGGTATTGTAATCTTGACGAATCTCTTCA | |
| | | | AGGAGAAGAAAGTCTGGTCTGCCAAAGCGCGT | |
| | | | TGGATCGTCTATGTAAAACCAAAGAACTTGAT | |
| | | | TACATGAGCTACGTTGTCCTAAGGCTGTTTGAG | |
| | | | AACACTGAGATATCTCTGGACGACCCGAAACG | |
| | | | ATTTAGAATCGAACTTACATTTAGCCGTGGCG | |
| | | | CAGATTTGTCTCCCTTAGAGAAGAAGGACGAG | |
| | | | GAAGCAGAATCATTGCTTCGAGAACACACACT | |
| | | | TCCGATAATGGGACCAGAAAGGCTTCAAGAGG | |
| | | | TTGGTTCATGTTTGACCCTCGAGACAATGGAG | |
| | | | AAGATGATACGTCCATTTGCAATGCCAGCCGA | |
| | | | GGATTTCCCTCCACCGTGTACTCCAGCAGGCTT | |
| | | | CTCCGGTTACTTCTCTAAAAGCGCCGCTGTTCT | |
| | | | CGAACGTCTTGTTAAACTTTGGCCCTTCCACAA | |
| | | | GAACACCTCTAATGGCAAAAGCTAAACGCCAA | |
| | | | CAAAGTGGGTGACTAATGACTAATGAATGATG | |
| | | | ATGATATTCCTAATCCATTTATTTTTTGAATCA | |
| | | | AAATTTTGAATTGTTAGTAGTTTCTAATTGAGT | |
| | | | ACACTAAATTGTTCATTTTGTTTTGTAGAATCG | |
| | | | TCAAAAAAAATTGTTAAAAACAATTGCTAATT | |
| | | | GATTTAGCATTATGGAAAATTTCCAAGCTTCTA | |
| | | | CTTGTCGTTTGCTTTGAT | |
| VIP1 | NM_110997 | 821297 | CCTGTGACGACTGAATTGTCCTCTGTCTGGCCC | 6 |
| (VIH2) | | (gene symbol | CGTGAGCTTCACCGCAGCTTTTAGATATTTTCT | |
| | | AT5G15070.2) | TTCCCCGTTCTTTCGGATTTTTTTTTTCTCCCCC | |
| | | | TCTCAAGCAAAGCATTATCGCTATTGCTCTGCC | |
| | | | TTGTGAGCTGCGATCACCTGTTTACTGAGAGA | |
| | | | AGAACACTGCGACGTCGTTAAAAGTAGGTTGA | |
| | | | GAGCGAGAAAGGCCGAGAGAATGGAGATGGA | |
| | | | AGAAGGAGCAAGTGGTGTTGGTGAGAAGATA | |
| | | | AAGATTGGAGTCTGCGTCATGGAAAAGAAGGT | |
| | | | GAAATGCGGCTCCGAGGTTTTCTCAGCTCCCAT | |
| | | | GGGGGAAATTCTCGACAGACTCCAGTCTTTTG | |
| | | | GTGAATTTGAGATCTTACATTTTGGGGATAAA | |
| | | | GTTATACTTGAAGATCCAATAGAAAGTTGGCC | |
| | | | CATTTGTGATTGCTTGATTGCTTTTCATTCCTCT | |
| | | | GGATATCCTCTTGAGAAGGCTCAGGCATATGC | |
| | | | TGCTTTAAGAAAGCCATTTTTAGTGAATGAACT | |
| | | | TGATCCGCAATATCTTCTTCATGATCGCCGGAA | |
| | | | GGTGTATGAGCATCTTGAGATGTATGGCATCC | |
| | | | CAGTTCCTAGGTATGCTTGTGTCAATAGAAAG | |
| | | | GTACCAAATCAAGACCTTCATTATTTTGTCGAG | |
| | | | GAAGAAGATTTTGTTGAGGTCCATGGTGAACG | |
| | | | CTTCTGGAAGCCATTTGTGGAAAAGCCTGTCA | |
| | | | ATGGAGATGACCATAGTATAATGATATACTAC | |
| | | | CCTAGCTCGGCAGGTGGAGGCATGAAAGAATT | |
| | | | GTTTCGCAAGATTGGGAACCGATCAAGTGAAT | |
| | | | TTCATCCTGACGTCAGAAGGGTAAGGAGAGAA | |
| | | | GGCTCTTATATATACGAGGAGTTTATGGCCAC | |
| | | | CGGAGGAACTGATGTCAAGGTCTATACAGTGG | |
| | | | GTCCTGAATACGCACATGCTGAAGCAAGAAAG | |
| | | | TCACCTGTTGTTGATGGTGTTGTTATGAGGAAT | |
| | | | ACAGATGGGAAGGAAGTGAGATATCCAGTATT | |
| | | | GCTTACACCTGCTGAAAAGCAAATGGCTAGAG | |
| | | | AAGTTTGCATTGCATTTAGGCAAGCGGTCTGT | |
| | | | GGGTTTGATCTTCTACGATCTGAGGGCTGTTCA | |
| | | | TATGTTTGTGATGTCAATGGATGGAGTTTTGTG | |
| | | | AAGAACTCATACAAGTATTATGACGATGCTGC | |
| | | | GTGTGTGCTAAGGAAAATGTGTTTGGATGCAA | |
| | | | AAGCTCCTCATCTCTCATCCACTCTTCCTCCCA | |
| | | | CCTTGCCATGGAAGGTCAATGAACCTGTACAA | |
| | | | TCTAATGAAGGTCTAACTCGCCAGGGAAGTGG | |
| | | | CATCATCGGCACTTTTGGGCAGTCAGAAGAGC | |
| | | | TACGTTGTGTCATTGCTGTTGTTCGACATGGCG | |
| | | | ATAGAACTCCCAAGCAGAAAGTGAAACTAAA | |
| | | | AGTTACAGAGGAAAAACTATTAAACCTAATGT | |
| | | | TGAAGTACAATGGTGGAAAGCCAAGAGCTGA | |
| | | | GACCAAACTTAAGAGTGCCGTCCAGTTGCAAG | |
| | | | ATCTATTAGATGCCACAAGAATGTTAGTTCCCC | |
| | | | GTACAAGACCAGGTCGTGAAAGTGATAGTGAT | |
| | | | GCAGAAGACCTTGAACATGCTGAGAAGCTTCG | |
| | | | GCAAGTTAAAGCAGTCCTTGAAGAGGGAGGAC | |
| | | | ATTTCTCTGGTATATACAGGAAGGTTCAACTG | |
| | | | AAGCCGCTGAAGTGGGTTAAAATACCAAAAAG | |
| | | | CGATGGTGATGGCGAAGAAGAAAGACCAGTA | |
| | | | GAGGCCCTTATGGTTCTGAAATATGGCGGTGT | |
| | | | TTTAACACACGCTGGTAGAAAGCAGGCAGAAG | |
| | | | AACTTGGTAGATACTTTCGAAACAATATGTAT | |
| | | | CCAGGTGAAGGGACTGGTTTGCTTCGTCTCCAT | |
| | | | AGTACGTACCGTCATGACCTTAAAATTTACAG | |
| | | | CTCTGACGAGGGACGTGTTCAGATGTCTGCAG | |
| | | | CGGCTTTTGCTAAAGGCCTGCTTGACCTAGAA | |
| | | | GGACAGCTGACGCCAATCCTGGTTTCTTTGGTT | |
| | | | AGCAAGGACTCTTCCATGTTGGATGGTCTTGAT | |
| | | | AATGCCAGCATTGAAATGGAAGCGGCCAAGGC | |
| | | | TAGATTGAATGAGATTGTAACGTCTGGCACAA | |
| | | | AGATGATAGACGACGACCAAGTCTCCTCTGAA | |
| | | | GATTTCCCTTGGATGACTGATGGAGCTGGACTT | |
| | | | CCTCCCAACGCTCATGAACTCCTACGTGAACT | |
| | | | GGTGAAATTAACTAAGAATGTGACTGAACAAG | |
| | | | TAAGACTACTTGCAATGGATGAAGACGAGAAC | |
| | | | CTCACTGAGCCATACGATATAATTCCTCCATAT | |
| | | | GATCAAGCAAAAGCCCTTGGCAAGACAAACAT | |
| | | | TGACAGTGATCGGATTGCTTCTGGATTACCATG | |
| | | | TGGTAGTGAAGGATTCCTTCTGATGTTTGCTCG | |
| | | | GTGGATAAAACTTGCTAGGGATCTCTACAATG | |
| | | | AAAGAAAAGACCGATTTGACATCACACAGATT | |
| | | | CCGGATGTTTACGATTCATGCAAGTACGACCT | |
| | | | GTTACATAATTCCCATCTCGATCTAAAAGGATT | |
| | | | AGATGAACTCTTCAAAGTAGCACAGTTACTTG | |
| | | | CAGATGGTGTAATCCCAAATGAGTATGGCATC | |
| | | | AATCCGCAACAAAAGCTTAAAATCGGTTCAAA | |
| | | | GATTGCTCGGCGCTTAATGGGGAAAATCTTGA | |
| | | | TAGACTTGAGGAATACTCGAGAAGAAGCACTG | |
| | | | AGCGTTGCCGAGTTGAAAGAAAGCCAAGAAC | |
| | | | AAGTCTTGTCATTATCCGCCTCGCAAAGGGAA | |
| | | | GATAGAAATAGTCAACCGAAGCTATTTATCAA | |
| | | | TAGCGATGAATTGAGACGACCTGGCACAGGTG | |
| | | | ATAAAGATGAAGATGATGATAAAGAAACCAA | |
| | | | ATACCGATTAGATCCAAAGTATGCAAATGTGA | |
| | | | AGACACCTGAACGTCATGTGAGGACTCGACTT | |
| | | | TACTTCACATCTGAATCGCATATTCATTCGCTC | |
| | | | ATGAACGTCCTTCGATACTGTAACCTTGATGA | |
| | | | ATCTCTCCTAGGAGAAGAAAGCCTCATTTGCC | |
| | | | AAAACGCCTTAGAACGTCTCTGTAAAACCAAG | |
| | | | GAACTCGATTACATGAGTTACATTGTCCTAAG | |
| | | | GCTCTTTGAGAACACCGAGGTATCACTTGAAG | |
| | | | ACCCGAAAAGATTCCGCATTGAACTTACATTT | |
| | | | AGCCGTGGAGCTGATTTGTCTCCCTTAAGGAA | |
| | | | TAATGACGACGAGGCAGAGACATTGCTAAGGG | |
| | | | AACACACACTTCCGATAATGGGACCAGAGAGG | |
| | | | CTTCAAGAAGTAGGTTCTTGTTTAAGCTTGGAG | |
| | | | ACAATGGAGAAAATGGTTCGTCCATTTGCTAT | |
| | | | GCCGGCGGAAGATTTCCCTCCGGCGTCCACTC | |
| | | | CGGTTGGTTTCTCGGGTTACTTCTCAAAAAGTG | |
| | | | CTGCGGTGCTGGAGCGACTCGTAAACCTCTTC | |
| | | | CACAACTATAAGAACTCTTCTTCTAATGGAAG | |
| | | | GAGCTAAGCAGAGACCAATTGTTTTTTCCACTT | |
| | | | ATATATATTTTGCTCATGCTTTTTTCTATGTGCA | |
| | | | GTTTTTAATCATCTTCTTGTTTTTCTACTGATGT | |
| | | | TTTAATTTTCTATGTGAAATTGTGAAAGGGACT | |
| | | | AGTGCAGTTGGGTTTGAATAAAGATATGATTA | |
| | | | CATGGAACATAAATGAGTCACCCGTGACGTTG | |
| | | | TCTAGTGTTTACAATAGGACTCATCTATTATAA | |
| | | | CTCAAGAAATGATGATGAGTCTTATTGTAAAC | |
| | | | ACTGTGTTTTCATATCATGCTGTTACGTTAAAG | |
| | | | TTGTTGCGGATTTGTTGGAATTTATATATAATC | |
| | | | TGCAAAAGAATAGGCTGATGATATCG | |

TABLE 4
Reference VIP proteins from Arabidopsis thaliana

| | Protein (GenBank | | SEQ |
|---|---|---|---|
| VIP | Accession No.) | Reference Sequence | ID NO: |
| VIP2 | NP_568308 | MGVEEGAGVDKKITIGVCVMEKKVFSAPMGQIMDRIHAFG | 7 |
| | | EFEIIHFGDKVILEDPVESWPICDCLIAFYSSGYPLEKVQAYSS | |
| | | LRKPFLVNELDPQYLLHDRRKVYEHLEMYGIPVPRYACVNR | |
| | | KVPDEDLDYFVEEEDFVEVKGERFWKPFVEKPVNGDDHSI | |
| | | MIYYPSSAGGGMKELFRKVGNRSSEFHPDVRRVRREGSYIY | |
| | | EEFMPTGGTDVKVYTVGPEYAHAEARKSPVVDGVVMRNP | |
| | | DGKEVRYPVLLTPAEKQMAREVCIAFRQAVCGFDLLRSEGS | |
| | | SYVCDVNGWSFVKNSYKYYDDAACVLRKMFLDAKAPHLS | |
| | | STIPPILPWKINEPVQSNEGLTRQGSGIIGTFGQSEELRCVIAIV | |
| | | RHGDRTPKQKVKLKVTEEKLLNLMLKYNGGKPRAETKLKT | |
| | | AVQLQDLLDATRMLIPRARSGESDSDAEDLEHADKLRQVK | |
| | | AVLEEGGHFSGIYRKVQLKPLKWVNVPKSDGEGEEERPVEA | |
| | | LMVLKYGGVLTHAGRKQAEELGRYFRNNMYPGEGTGLLR | |
| | | LHSTYRHDLKIYSSDEGRVQMSAAAFAKGLLDLEGQLTPIL | |
| | | VSLVSKDSSMLDGLDNASSEMEAAKAQLNEIITAGSKMVHD | |
| | | HVSSELPWMTDGAGLPPHADEHLPELVKLAKKVTEQVRLL | |
| | | AQDEHENLAEPSAYDVVPPYDQAKALGKSNIDVGRIAAGLP | |
| | | CGSEGFLLMFARWRKLERDLYNERRERFDITQIPDVYDSCK | |
| | | YDLLHNSHLDLKGLDELFKVAQLLADGVIPNEYGINPQQKL | |
| | | KIGSKIARRLLGKILIDLRNTREEAMSVAELKNSQDQVSVSL | |
| | | YSSRKEDRYSQPKLFVKSDELRRPSTGENKEEDDDKETKYR | |
| | | LDPKYANVMTPERHVRTRLYFTSESHIHSLMNVLRYCNLDE | |
| | | SLQGEESLVCQSALDRLCKTKELDYMSYVVLRLFENTEISLD | |
| | | DPKRFRIELTFSRGADLSPLEKKDEEAESLLREHTLPIMGPER | |
| | | LQEVGSCLTLETMEKMIRPFAMPAEDFPPPCTPAGFSGYFSK | |
| | | SAAVLERLVKLWPFHKNTSNGKS | |
| VIP1 | NP_186780 | MEMEEGASGVGEKIKIGVCVMEKKVKCGSEVFSAPMGEILD | 8 |
| | | RLQSFGEFEILHFGDKVILEDPIESWPICDCLIAFHSSGYPLEK | |
| | | AQAYAALRKPFLVNELDPQYLLHDRRKVYEHLEMYGIPVP | |
| | | RYACVNRKVPNQDLHYFVEEEDFVEVHGERFWKPFVEKPV | |
| | | NGDDHSIMIYYPSSAGGGMKELFRKIGNRSSEFHPDVRRVR | |
| | | REGSYIYEEFMATGGTDVKVYTVGPEYAHAEARKSPVVDG | |
| | | VVMRNTDGKEVRYPVLLTPAEKQMAREVCIAFRQAVCGFD | |
| | | LLRSEGCSYVCDVNGWSFVKNSYKYYDDAACVLRKMCLD | |
| | | AKAPHLSSTLPPTLPWKVNEPVQSNEGLTRQGSGIIGTFGQS | |
| | | EELRCVIAVVRHGDRTPKQKVKLKVTEEKLLNLMLKYNGG | |
| | | KPRAETKLKSAVQLQDLLDATRMLVPRTRPGRESDSDAEDL | |
| | | EHAEKLRQVKAVLEEGGHFSGIYRKVQLKPLKWVKIPKSDG | |
| | | DGEEERPVEALMVLKYGGVLTHAGRKQAEELGRYFRNNM | |
| | | YPGEGTGLLRLHSTYRHDLKIYSSDEGRVQMSAAAFAKGLL | |
| | | DLEGQLTPILVSLVSKDSSMLDGLDNASIEMEAAKARLNEIV | |
| | | TSGTKMIDDDQVSSEDFPWMTDGAGLPPNAHELLRELVKLT | |
| | | KNVTEQVRLLAMDEDENLTEPYDIIPPYDQAKALGKTNIDS | |
| | | DRIASGLPCGSEGFLLMFARWIKLARDLYNERKDRFDITQIP | |
| | | DVYDSCKYDLLHNSHLDLKGLDELFKVAQLLADGVIPNEY | |
| | | GINPQQKLKIGSKIARRLMGKILIDLRNTREEALSVAELKESQ | |
| | | EQVLSLSASQREDRNSQPKLFINSDELRRPGTGDKDEDDDKE | |
| | | TKYRLDPKYANVKTPERHVRTRLYFTSESHIHSLMNVLRYC | |
| | | NLDESLLGEESLICQNALERLCKTKELDYMSYIVLRLFENTE | |
| | | VSLEDPKRFRIELTFSRGADLSPLRNNDDEAETLLREHTLPIM | |
| | | GPERLQEVGSCLSLETMEKMVRPFAMPAEDFPPASTPVGFS | |
| | | GYFSKSAAVLERLVNLFHNYKNSSSNGRS | |

In some embodiments, the VIP gene or gene product contains or is composed completely of a polynucleotide. In some embodiments, the VIP gene or gene product contains or is completely composed of a sequence that is about 70% to 100% identical to any one of SEQ ID NO: 5-6 and/or a sequence complementary thereto. In some embodiments, the VIP gene or gene product contains or is completely composed of a sequence that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 5-6 and/or a sequence complementary thereto.

In some embodiments, the VIP gene product contains or is composed completely of a polypeptide. In some embodiments, the VIP gene product contains or is completely composed of a sequence that is about 70% to 100% identical to any one of SEQ ID NO: 7-8. In some embodiments, the VIP gene product contains or is completely composed of a sequence that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 7-8. In some embodiments, the gene encodes only the kinase domain of a VIP gene. In some embodiments, the VIP gene product is a VIP kinase domain. In some embodiments, the VIP kinase domain comprises or is composed only of amino acids 10-337 of SEQ ID NO: 8 or amino acids corresponding thereto in a variant, homologue, orthologue, or paralogue thereof. In some embodiments, the VIP kinase domain comprises or is composed entirely of a polypeptide that is about 70% to 100% identical to amino acids 10-337 of SEQ ID NO: 8 or amino acids corresponding thereto in a variant, homologue, orthologue, or paralogue thereof. In some embodiments, the VIP kinase domain comprises or is composed entirely of a polypeptide that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to amino acids 11-338 of SEQ ID NO: 7 or amino acids corresponding thereto in a variant, homologue, orthologue, or paralogue thereof. In some embodiments, the VIP gene product is a VIP kinase domain. In some embodiments, the VIP kinase domain comprises or is composed only of amino acids 11-338 of SEQ ID NO: 7 or amino acids corresponding thereto in a variant, homologue, orthologue, or paralogue thereof. In some embodiments, the VIP kinase domain comprises or is composed entirely of a polypeptide that is about 70% to 100% identical to amino acids 11-338 of SEQ ID NO: 7 or amino acids corresponding thereto in a variant, homologue, orthologue, or paralogue thereof. In some embodiments, the VIP kinase domain comprises or is composed entirely of a polypeptide that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to amino acids 11-338 of SEQ ID NO: 7 or amino acids corresponding thereto in a variant, homologue, orthologue, or paralogue thereof.

### Vectors and Vector Systems

Also provided herein are vectors that can contain one or more of the ITPK and/or VIP encoding polynucleotides described herein. The vectors can be useful in producing, for example, bacterial, plant cells, and/or transgenic (engineered) plants that can contain, replicate, and/or express an ITPK and/or VIP gene and/or gene product described herein. Within the scope of this disclosure are vectors containing one or more of the polynucleotide sequences described herein. One or more of the polynucleotides that are part of the ITPK and/or VIP encoding polynucleotides described herein can be included in a vector or vector system. The vectors and/or vector systems can be used, for example, to express one or more of the polynucleotides in a cell, such as a plant cell or Agrobacterium or to produce particles such as viral particles that can be used to generate transgenic cells and/or plants. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for expression one or more ITPK and/or VIP encoding polynucleotides or other (e.g., reporter) polynucleotides and/or one or more regions or domains thereof described herein.

### Cell-Based Vector Amplification and Expression

Vectors can be designed for amplification, propagation, and expression of one or more elements of the ITPK and/or VIP encoding polynucleotides described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, mammalian cells, and more particularly plant cells. The vectors can be viral-based or non-viral based. In some embodiments, the suitable host cell is a eukaryotic cell. In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to, Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, where one or more ITPK and/or VIP encoding polynucleotides, functional domains thereof, and/or one or more additional polynucleotides are included and/or expressed (e.g., a reporter polynucleotide), each be operably linked to separate regulatory elements on the same or separate vectors. In some embodiments, two or more of the elements expressed from the same or different regulatory element(s), can be combined in a single vector, with one or more additional vectors providing any components of the system not included in the first vector. Encoding polynucleotides (ITPK and/or VIP) that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more ITPK and/or VIP proteins and/or functional domains thereof and/or one or more other genes (e.g., reporter gene), embedded within one or more intron sequences (e.g., each in a different intron, two or more in at least one intron, or all in a single intron). In some embodiments, the ITPK and/or VIP proteins and/or functional domains thereof and/or one or more other genes (e.g., reporter gene) can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

Where a vector or vector system is provided that contains an ITPK and/or VIP encoding polynucleotide, the encoding polynucleotide can be operatively coupled to one or more regulatory elements. In some embodiments, the regulatory element can drive ubiquitous expression. In some embodiments, the regulatory element can drive or control cell or tissue specific expression. In some embodiments, the regulatory element can drive conditional or inducible expression. In some embodiments, the ITPK and/or VIP encoding polynucleotide is operatively coupled to a plant promoter.

In embodiments, the polynucleotides and/or vectors thereof described herein (such as the ITPK and/or VIP encoding polynucleotides) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as leaves, stem, fruit, flower, etc., or particular cell types. Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the ITPK and/or VIP encoding polynucleotides described herein are typically placed under control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the ITPK and/or VIP encoding polynucleotides are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in expression of the ITPK and/or VIP encoding polynucleotides are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Further exemplary plant promoters suitable to drive expression of the VIP and/or ITPK gene product encoding polynucleotides include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome)., such as a light Inducible Transcriptional Effector (LITE) that direct changes in transcriptional activity in a sequence-specific manner. The components of a light inducible system may include one or more ITPK and/or VIP encoding polynucleotides described herein, a light-responsive cytochrome heterodimer (e.g., from Arabidopsis thaliana), and a transcriptional activation/repression domain. In some embodiments, the vector can include one or more of the inducible DNA binding proteins provided in PCT publication WO 2014/018423 and US Publications, 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

Where transient expression of an encoding polynucleotide described herein is desired, transient expression may be achieved using suitable vectors. Exemplary vectors that may be used for transient expression include a pEAQ vector (may be tailored for Agrobacterium-mediated transient expression) and Cabbage Leaf Curl virus (CaLCuV), and vectors described in Sainsbury F. et al., Plant Biotechnol J. 2009 September; 7(7):682-93; and Yin K et al., Scientific Reports volume 5, Article number: 14926 (2015).

A plant promoter is capable of initiating transcription in plant cells, whether or not its origin is a plant cell. The use of different types of promoters is envisaged.

In some embodiments, the vector or system thereof can include one or more elements capable of translocating and/or expressing an ITPK and/or VIP encoding polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc.

### Selectable Markers and Tags

One or more of the ITPK and/or VIP encoding polynucleotides can be operably linked, fused to, or otherwise modified to include a polynucleotide that encodes or is a selectable marker or tag, which can be a polynucleotide or polypeptide. In some embodiments, the polynucleotide encoding a polypeptide selectable marker can be incorporated with the ITPK and/or VIP encoding polynucleotides polynucleotide such that the selectable marker polypeptide, when translated, is inserted between two amino acids between the N- and C-terminus of the ITPK and/or VIP polypeptide or at the N- and/or C-terminus of the ITPK and/or VIP polypeptide. In some embodiments, the selectable marker or tag is a polynucleotide barcode or unique molecular identifier (UMI).

It will be appreciated that the polynucleotide encoding such selectable markers or tags can be incorporated into an ITPK and/or VIP encoding polynucleotide described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly (NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more ITPK and/or VIP polypeptides described herein via suitable linker, such as a glycine or glycine serine linkers which are known in the art.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organelles, etc. In some embodiments, the targeting moiety encoding polynucleotides can be included in the vector or vector system such that the ITPK and/or VIP polynucleotides and/or products expressed therefrom include the targeting moiety and can be targeted to specific cells, tissues, organelles, etc. In some embodiments, such as non-viral carriers, the targeting moiety can be attached to the carrier (e.g., polymer, lipid, inorganic molecule etc.) and can be capable of targeting the carrier and any attached or associated ITPK and/or VIP polynucleotide(s) to specific cells, tissues, organelles, etc.

### Cell-Free Vector and Polynucleotide Expression

In some embodiments, the polynucleotide encoding an ITPK and/or VIP gene product is expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg₂₊, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the ITPK and/or VIP gene product encoding polynucleotides described herein can be codon optimized. In some embodiments, one or more polynucleotides contained in a vector (“vector polynucleotides”) described herein that are in addition to an optionally codon optimized the ITPK and/or VIP gene product encoding polynucleotides described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some embodiments, the vector is a non-viral vector or carrier. In some embodiments, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with a heterologous ITPK and/or VIP encoding polynucleotide and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid, polynucleotide molecule, or composition that be attached to or otherwise interact with, encapsulate, and/or associate with a polynucleotide to be delivered, such as an ITPK and/or VIP gene product encoding polynucleotide of the present invention.

### Naked Polynucleotides

In some embodiments, one or more ITPK and/or VIP gene product encoding polynucleotides described elsewhere herein can be included in and/or delivered as a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the ITPK and/or VIP gene product encoding polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the ITPK and/or VIP gene product encoding polynucleotide(s) described herein. In some embodiments, the naked polynucleotide can contain other nucleic acids and/or polynucleotides in addition to the ITPK and/or VIP gene product encoding polynucleotide(s) described herein. The naked polynucleotides can include one or more elements of a transposon system. Transposons and system thereof are described in greater detail elsewhere herein.

### Non-Viral Polynucleotide Vectors

In some embodiments, one or more of the ITPK and/or VIP gene product encoding polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g., minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, Agrobacterium vectors (Ti or Ri vectors), PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In embodiments, the S/MAR sequence is located downstream of an actively transcribed polynucleotide (e.g., one or more ITPK and/or VIP gene product encoding polynucleotides described herein) included in the non-viral polynucleotide vector. In some embodiments, the S/MAR can be a S/MAR from the beta-interferon gene cluster. See e.g., Verghese et al. 2014. Nucleic Acid Res. 42: e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the ITPK and/or VIP gene product encoding polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the ITPK and/or VIP gene product encoding polynucleotide(s)) and integrate it into one or more positions in the host cell's genome. In some embodiments, the transposon vector or system thereof can be configured as a gene trap. In some embodiments, the TIRs can be configured to flank a strong splice acceptor site followed by a reporter and/or other gene (e.g., one or more of the ITPK and/or VIP gene product encoding polynucleotide(s)) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, without limitation, Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

In some embodiments, the non-viral vector or vector system is an Agrobacterium vector or vector system. In some embodiments, the ITPK and/or VIP gene product encoding polynucleotide(s) is included in a T-DNA (or Ti) vector or an Ri vector (See e.g., Gelvin, S. 2003. Microbiol Mol Biol Rev. 2003 March; 67(1): 16-37, particularly at FIG. 1A; Lee and Gelvin. Plant Physiol. 2008 February; 146(2): 325-332 and as described elsewhere herein.

### Chemical Carriers

In some embodiments, the ITPK and/or VIP gene product encoding polynucleotide(s) can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the ITPK and/or VIP gene product encoding polynucleotide(s)), (2) those capable of targeting specific cells, (3) those capable of increasing delivery of the polynucleotide (such as the ITPK and/or VIP gene product encoding polynucleotide(s)) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the ITPK and/or VIP gene product encoding polynucleotide(s) described herein. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticuloendothelial system. In some embodiments, the inorganic particles can be optimized to protect an entrapped molecule from degradation. The suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, particles, and materials, (e.g., supermagnetic iron oxide and magnetite), quantum dots, fullerenes (e.g., carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g., such as an ITPK and/or VIP gene product encoding polynucleotide). In some embodiments, chemical non-viral carrier systems can include a polynucleotide, such as the ITPK and/or VIP gene product encoding polynucleotide(s), and a lipid (such as a cationic lipid). These are also referred to in the art as lipoplexes. Other embodiments of lipoplexes are described elsewhere herein. In some embodiments, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g., the ITPK and/or VIP gene product encoding polynucleotide(s). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g., polymer-based carriers and lipid-based carriers to functionalize these carriers). In some embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the ITPK and/or VIP gene product encoding polynucleotide(s), polymethacrylate, and combinations thereof.

In some embodiments, the non-viral carrier can be configured to release an engineered delivery system polynucleotide that is associated with or attached to the non-viral carrier in response to an external stimulus, such as pH, temperature, osmolarity, concentration of a specific molecule or composition (e.g., calcium, NaCl, and the like), pressure and the like. In some embodiments, the non-viral carrier can be a particle that is configured includes one or more of the ITPK and/or VIP gene product encoding polynucleotide(s) described herein and an environmental triggering agent response element, and optionally a triggering agent. In some embodiments, the particle can include a polymer that can be selected from the group of polymethacrylates and polyacrylates. In some embodiments, the non-viral particle can include one or more embodiments of the compositions microparticles described in US Pat. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the ITPK and/or VIP gene product encoding polynucleotide(s)).

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as an ITPK and/or VIP gene product encoding polynucleotide, into a virus particle and producing said virus particle when used alone or with one or more other viral vectors (such as in a viral vector system). Viral vectors and systems thereof can be used for producing viral particles for delivery of and/or expression of one or more components of the ITPK and/or VIP gene product encoding polynucleotide described herein. The viral vector can be part of a viral vector system involving multiple vectors. In some embodiments, systems incorporating multiple viral vectors can increase the safety of these systems. Suitable viral vectors can include any plant viral vector or system such as any of those set forth in e.g., K. Hefferon. Biomedicines. Zaidi* and Mansoor. 2017 September; 5(3): 44, Front. Plant Sci., 11 Apr. 2017. https://doi.org/10.3389/fpls.2017.00539, and Abrahamian et al. 2020. Ann. Rev. Virol. 7:513-535, particularly those based on tobacco mosaic virus (TMV), Potexviruses, and Comovirus Cowpea mosaic virus (CPMV). Other embodiments of viral vectors and viral particles produce therefrom are described elsewhere herein. In some embodiments, the viral vectors are configured to produce replication incompetent viral particles for improved safety of these systems.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Virus Particle Production from Viral Vectors

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., an ITPK and/or VIP gene product encoding polynucleotide), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., ITPK and/or VIP transcripts, proteins, enzymes, mutant forms thereof, fusion proteins thereof, etc.), and virus particles (such as from viral vectors and systems thereof).

One or more ITPK and/or VIP gene product encoding polynucleotides can be delivered using an engineered plant virus particle containing the one or more ITPK and/or VIP gene product encoding polynucleotides using appropriate formulations and doses.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Engineered Plants Having Increased ITPK and/or VIP Gene Expression

As previously described in some embodiments the engineered plant has increased ITPK and/or VIP gene expression. In some embodiments, the engineered plant contains one or more exogenous ITPK and/or VIP genes or portion thereof that encodes an ITPK and/or VIP polypeptide, contains one or more modified endogenous ITPK and/or VIP genes; contains a modified epigenome thereby increasing ITPK and/or VIP gene expression; or any combination thereof. Methods and techniques of producing the engineered plants are discussed elsewhere herein.

Engineered Plants Expressing Exogenous ITPK and/or VIP Gene(s)

In some embodiments, the engineered plant contains one or more exogenous ITPK and/or VIP genes or portion thereof that encodes an ITPK and/or VIP polypeptide. The one or more exogenous ITPK and/or VIP genes or portion thereof that encodes an ITPK and/or VIP polypeptide can be stably integrated into the genome of one or more cells in the plant. The one or more exogenous ITPK and/or VIP genes or portion thereof that encodes an ITPK and/or VIP polypeptide can be transiently expressed of one or more cells in the plant. It will be appreciated that the exogenous ITPK and/or VIP gene can be an ITPK and/or VIP gene that is not natively expressed in the plant into which it is introduced or be an extra copy that is identical to or a modified (e.g., mutant) variant of an ITPK and/or VIP gene that is natively expressed in the plant into which it is introduced. In some embodiments, the engineered plant having increased ITPK and/or VIP enzyme activity has and/or expresses one or more exogenous ITPK and/or VIP genes.

In some embodiments, the exogenous ITPK and/or VIP gene expression construct that is contained and/or expressed in the one or more cells of the engineered plant comprises one or more ITPK and/or VIP genes or portions thereof encoding the one or more ITPK and/or VIP polypeptides operatively coupled to one or more exogenous promoters and/or regulator elements that drive and/or regulate expression of the one or more ITPK and/or VIP genes and/or portions thereof encoding one or more ITPK and/or VIP polypeptides. In some embodiments, the one or more exogenous promoters is a constitutive promoter. In some embodiments, the one or more exogenous promoters is a cell or tissue specific promoter. In some embodiments, the one or more exogenous promoters is a temporal promoter, i.e., a promoter that expressed only during certain time (e.g., times of day, year, or moth, or during certain developmental or production stages). In some embodiments, the promoter is an inducible promoter. Exemplary promoters and other regulatory elements that can be coupled to the one or more exogenous ITPK and/or VIP genes are described elsewhere herein.

In some embodiments, the exogenous ITPK and/or VIP gene expression construct that is contained and/or expressed in the one or more cells of the engineered plant does not contain a promoter and/or regulator elements. In some embodiments one or more exogenous ITPK and/or VIP genes or portion thereof that encodes an ITPK and/or VIP polypeptide are introduced into a genome of one or more cells in a location such that the expression of the exogenous ITPK and/or VIP gene is driven and/or otherwise regulated by promoter(s) and/or other regulatory elements endogenous to the engineered plant. For example, in some embodiments, the one or more exogenous ITPK and/or VIP genes or portion thereof that encodes an ITPK and/or VIP polypeptide are introduced such that the exogenous ITPK and/or VIP gene expression is driven off of and/or regulated by an endogenous ITPK and/or VIP promoter and/or endogenous regulator elements. In such embodiments, the exogenous ITPK and/or VIP gene can replace or be integrated in-frame with one or more endogenous ITPK and/or VIP genes. In embodiments, where the one or more exogenous ITPK and/or VIP genes or portion thereof that encodes an ITPK and/or VIP polypeptide are introduced such they are driven off a non-ITPK and/or VIP promoter and/or other regulator elements, the one or more exogenous ITPK and/or VIP genes or portion thereof that encodes an ITPK and/or VIP polypeptide can be introduced such that they replace one or more non-ITPK and/or VIP genes or are integrated in-frame with the one or more non-ITPK and/or VIP genes such that the expression of the exogenous ITPK and/or VIP gene or portion thereof that encodes an ITPK, VIP, and/or PSR polypeptide is driven off of and/or regulated by the promoter and/or other regulator elements that drive and/or regulate expression of the non-ITPK and/or VIP endogenous gene(s).

Engineered Plants Expressing Modified Endogenous ITPK, VIP, and/or PSR Gene(s)

In some embodiments, the engineered plant contains one or more modified endogenous ITPK and/or VIP genes comprise one or more mutations, insertions, deletions, substitutions, or any combination thereof in the coding or non-coding region of the one or more modified endogenous ITPK and/or VIP genes such that expression of the one or more modified endogenous ITPK and/or VIP genes are increased as compared to an unmodified control. In some embodiments, the engineered plant having increased ITPK and/or VIP enzyme or other activity has a one or more modified endogenous ITPK and/or VIP genes.

In some embodiments, an insertion, deletion, or indel in a modified endogenous ITPK and/or VIP gene ranges in size from 1-50 or more base pairs. In some embodiments, an insertion, deletion, or indel in a modified endogenous ITPK and/or VIP gene is 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more.

In some embodiments, the modified ITPK and/or VIP gene or ITPK and/or VIP gene product encoding polynucleotide has 1-500 or more mutated or substituted bases or base pairs In some embodiments, the modified ITPK and/or VIP gene or gene product encoding polynucleotide has 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 or more mutated or substituted bases or base pairs.

Methods and techniques for introducing mutations, insertions, deletions, substitutions, or any combination thereof are generally known in the art and described elsewhere herein. Such methods and techniques include, but are not limited to, recombinant engineering and genome editing via programmable nucleases (e.g., TALENs, Zinc Finger nuclease, CRISPR-Cas systems, CRISPR-Cas based systems (e.g., CRISPR-associated transposases (CAST), and Primer Editors, and/or the like). See e.g., Malzahn et al., Cell Biosci. 2017 Apr. 24; Molla et al., Nat Plants. 2021. PMID: 34518669k 7:21. doi: 10.1186/s13578-017-0148-4. eCollection 2017; Zhang et al., Prog Mol Biol Transl Sci. 2017; 149:133-150. doi: 10.1016/bs.pmbts.2017.03.008; Sprink et al., Curr Opin Biotechnol. 2015 April; 32:47-53. doi: 10.1016/j.copbio.2014.11.010; Razzaq et al., Int J Mol Sci. 2019 Aug. 19; 20(16):4045. doi: 10.3390/ijms20164045; Sanagala et al., Sanagala R, et al. J Genet Eng Biotechnol. 2017. PMID: 306476; Norman et al., Front Plant Sci. 2016. PMID: 27917188; Molka et al., Strecker et al., Science. 2019. 365(6448):48-53; and Nandy et al., J Biosci. 2020. PMID: 32020912, which can be adapted for use with the present disclosure. In view of at least the reference sequences in the context of the disclosure herein, one of ordinary skill in the art can apply any one or more of these methods and techniques to generate engineered plants having one or more modified endogenous ITPK and/or VIP genes and/or gene products. In some embodiments, such as when the engineered plant is generated using a CRISPR-Cas system or CRISPR-based system, the engineered plant comprises a CRISPR-Cas system or component(s) thereof.

### Engineered Plants Having a Modified Epigenome

In some embodiments the engineered has a modified epigenome. Epigenome modification refers to the modification of an epigenetic landscape local to an endogenous genomic site of interest. In the context of the instant description, the epigenetic landscape modified is local to the genomic site of one or more endogenous ITPK and/or VIP genes. Without being bound by theory, by modifying the epigenome local to the genomic of one or more endogenous ITPK and/or VIP genes. The term “local to” as used in this context herein includes the site of the gene within the genome as well as the region that is in proximity to the gene such that modification of the epigenome in that region can modify (increase or decrease) the accessibility of the genomic site of the gene or regulatory region thereof. In some embodiments, modifying the epigenetic landscape local to the genomic site of the one or more ITPK and/or VIP genes increases expression of the one or more ITPK and/or VIP genes. Without being bound by theory, the modification(s) to the epigenetic landscape local to the genomic site of the one or more ITPK and/or VIP genes increases accessibility to the one or more ITPK and/or VIP genes such that transcription is increased. In some embodiments, the modification of the epigenetic landscape local to the genomic site of the one or more ITPK and/or VIP genes is DNA demethylation, histone demethylation, histone acetylation, or any combination thereof. In some embodiments, the engineered plant having increased ITPK and/or VIP enzyme or other activity has a modified epigenetic landscape local to the genomic site of one or more endogenous ITPK and/or VIP genes.

In some embodiments, DNA methylation of one or more of the ITPK and/or VIP genes, regions thereof, or regulatory region(s) thereof is decreased by 0.01-100%. In some embodiments DNA methylation of one or more of the ITPK and/or VIP genes, regions thereof, or regulatory region(s) thereof is decreased by 0.01%, to/or 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99% 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%.

In some embodiments, histone methylation at an epigenomic site local to one or more of the ITPK and/or VIP genes, regions thereof, or regulatory region(s) thereof is decreased by 0.01-100%. In some embodiments, histone methylation at an epigenomic site local to one or more of the ITPK and/or VIP genes, regions thereof, or regulatory region(s) thereof is decreased by 0.01%, to/or 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99% 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%.

In some embodiments, histone acetylation at an epigenomic site local to one or more of the ITPK and/or VIP genes, regions thereof, or regulatory region(s) thereof is increased by 0.01-100 fold or more. In some embodiments, histone acetylation at an epigenomic site local to one or more of the ITPK and/or VIP genes, regions thereof, or regulatory region(s) thereof is increased by 0.01, to/or 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 fold or more.

In some embodiments, histone acetylation at an epigenomic site local to one or more of the ITPK and/or VIP genes, regions thereof, or regulatory region(s) thereof is increased by about 1 to 1000 percent or more. In some embodiments, histone acetylation at an epigenomic site local to one or more of the ITPK and/or VIP genes, regions thereof, or regulatory region(s) thereof is increased by about 1%, to/or 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, 501%, 502%, 503%, 504%, 505%, 506%, 507%, 508%, 509%, 510%, 511%, 512%, 513%, 514%, 515%, 516%, 517%, 518%, 519%, 520%, 521%, 522%, 523%, 524%, 525%, 526%, 527%, 528%, 529%, 530%, 531%, 532%, 533%, 534%, 535%, 536%, 537%, 538%, 539%, 540%, 541%, 542%, 543%, 544%, 545%, 546%, 547%, 548%, 549%, 550%, 551%, 552%, 553%, 554%, 555%, 556%, 557%, 558%, 559%, 560%, 561%, 562%, 563%, 564%, 565%, 566%, 567%, 568%, 569%, 570%, 571%, 572%, 573%, 574%, 575%, 576%, 577%, 578%, 579%, 580%, 581%, 582%, 583%, 584%, 585%, 586%, 587%, 588%, 589%, 590%, 591%, 592%, 593%, 594%, 595%, 596%, 597%, 598%, 599%, 600%, 601%, 602%, 603%, 604%, 605%, 606%, 607%, 608%, 609%, 610%, 611%, 612%, 613%, 614%, 615%, 616%, 617%, 618%, 619%, 620%, 621%, 622%, 623%, 624%, 625%, 626%, 627%, 628%, 629%, 630%, 631%, 632%, 633%, 634%, 635%, 636%, 637%, 638%, 639%, 640%, 641%, 642%, 643%, 644%, 645%, 646%, 647%, 648%, 649%, 650%, 651%, 652%, 653%, 654%, 655%, 656%, 657%, 658%, 659%, 660%, 661%, 662%, 663%, 664%, 665%, 666%, 667%, 668%, 669%, 670%, 671%, 672%, 673%, 674%, 675%, 676%, 677%, 678%, 679%, 680%, 681%, 682%, 683%, 684%, 685%, 686%, 687%, 688%, 689%, 690%, 691%, 692%, 693%, 694%, 695%, 696%, 697%, 698%, 699%, 700%, 701%, 702%, 703%, 704%, 705%, 706%, 707%, 708%, 709%, 710%, 711%, 712%, 713%, 714%, 715%, 716%, 717%, 718%, 719%, 720%, 721%, 722%, 723%, 724%, 725%, 726%, 727%, 728%, 729%, 730%, 731%, 732%, 733%, 734%, 735%, 736%, 737%, 738%, 739%, 740%, 741%, 742%, 743%, 744%, 745%, 746%, 747%, 748%, 749%, 750%, 751%, 752%, 753%, 754%, 755%, 756%, 757%, 758%, 759%, 760%, 761%, 762%, 763%, 764%, 765%, 766%, 767%, 768%, 769%, 770%, 771%, 772%, 773%, 774%, 775%, 776%, 777%, 778%, 779%, 780%, 781%, 782%, 783%, 784%, 785%, 786%, 787%, 788%, 789%, 790%, 791%, 792%, 793%, 794%, 795%, 796%, 797%, 798%, 799%, 800%, 801%, 802%, 803%, 804%, 805%, 806%, 807%, 808%, 809%, 810%, 811%, 812%, 813%, 814%, 815%, 816%, 817%, 818%, 819%, 820%, 821%, 822%, 823%, 824%, 825%, 826%, 827%, 828%, 829%, 830%, 831%, 832%, 833%, 834%, 835%, 836%, 837%, 838%, 839%, 840%, 841%, 842%, 843%, 844%, 845%, 846%, 847%, 848%, 849%, 850%, 851%, 852%, 853%, 854%, 855%, 856%, 857%, 858%, 859%, 860%, 861%, 862%, 863%, 864%, 865%, 866%, 867%, 868%, 869%, 870%, 871%, 872%, 873%, 874%, 875%, 876%, 877%, 878%, 879%, 880%, 881%, 882%, 883%, 884%, 885%, 886%, 887%, 888%, 889%, 890%, 891%, 892%, 893%, 894%, 895%, 896%, 897%, 898%, 899%, 900%, 901%, 902%, 903%, 904%, 905%, 906%, 907%, 908%, 909%, 910%, 911%, 912%, 913%, 914%, 915%, 916%, 917%, 918%, 919%, 920%, 921%, 922%, 923%, 924%, 925%, 926%, 927%, 928%, 929%, 930%, 931%, 932%, 933%, 934%, 935%, 936%, 937%, 938%, 939%, 940%, 941%, 942%, 943%, 944%, 945%, 946%, 947%, 948%, 949%, 950%, 951%, 952%, 953%, 954%, 955%, 956%, 957%, 958%, 959%, 960%, 961%, 962%, 963%, 964%, 965%, 966%, 967%, 968%, 969%, 970%, 971%, 972%, 973%, 974%, 975%, 976%, 977%, 978%, 979%, 980%, 981%, 982%, 983%, 984%, 985%, 986%, 987%, 988%, 989%, 990%, 991%, 992%, 993%, 994%, 995%, 996%, 997%, 998%, 999%, 1000% or more.

Epigenome editing can be accomplished utilizing a programmable nuclease system or other approach to provide targeted epigenomic modification and/or delivery of a demethylase, acetylase, and/or acetyl transferase to the epigenomic site local to the endogenous genomic site of interest (e.g., an endogenous ITPK and/or VIP gene or regulator region thereof). Methods and techniques of epigenomic modification are generally known in the art as is described in e.g., Thakore et al., Nat Methods. 2016 February; 13(2):127-37. doi: 10.1038/nmeth.3733; Nakamura et al., Nat Cell Biol. 2021 January; 23(1):11-22. doi: 10.1038/s41556-020-00620-7; Brocken et al., Curr Issues Mol Biol. 2018; 26:15-32. doi: 10.21775/cimb.026.015; Holtzman and Gersbach. Annu Rev Genomics Hum Genet. 2018 Aug. 31; 19:43-71. doi: 10.1146/annurev-genom-083117-021632; Rots and Jeltsch. Methods Mol Biol. 2018; 1767:3-18. doi: 10.1007/978-1-4939-7774-1_1; Waryah et al., Methods Mol Biol. 2018; 1767:19-63. doi: 10.1007/978-1-4939-7774-1_2; Gjaltema and Rots. Curr Opin Chem Biol. 2020 August; 57:75-81. doi: 10.1016/j.cbpa.2020.04.020; Enriquex, P., Yale J Biol Med. 2016 Dec. 23; 89(4):471-48; Goell and Hilton. Trends Biotechnol. 2021 July; 39(7):678-691. doi: 10.1016/j.tibtech.2020.10.012; Pei et al., Brief Funct Genomics. 2020 May 20; 19(3):215-228. doi: 10.1093/bfgp/elz035; Zhao et al., Int J Mol Sci. 2020 Feb. 3; 21(3):998. doi: 10.3390/ijms21030998; Gomez et al., Trends Genet. 2019 July; 35(7):527-541. doi: 10.1016/j.tig.2019.04.007; Lau et al., Transgenic Res. 2018 December; 27(6):489-509. doi: 10.1007/s11248-018-0096-8; Kungulovski et al., Trends Genet. 2016 February; 32(2):101-113. doi: 10.1016/j.tig.2015.12.001; Chakravarti et al., Curr Drug Targets. 2022; 23(8):836-853. doi: 10.2174/1389450123666220117105531; Laufer et al., Epigenetics Chromatin. 2015 Sep. 17; 8:34. doi: 10.1186/s13072-015-0023-7; Bozorg et al., Adv Biomed Res. 2019 Aug. 21; 8:49. doi: 10.4103/abr.abr_41_19. eCollection 2019; Moradpour and Abdulah et al., Plant Biotechnol J. 2020 January; 18(1):32-44. doi: 10.1111/pbi.13232. Epub 2019; Kubik et al., Chembiochem. 2016 Jun. 2; 17(11):975-80. doi: 10.1002/cbic.201600072. Epub 2016 Apr. 20; Zhan et al., J Integr Plant Biol. 2021 January; 63(1):3-33. doi: 10.1111/jipb.13063; Dubois and Roudier. Epigenomes. 2021 Aug. 24; 5(3):17. doi: 10.3390/epigenomes5030017; Dhakate et al., Front Genet. 2022 Aug. 23; 13:876987. doi: 10.3389/fgene.2022.876987. eCollection 2022; Selma et al., Transgenic Res. 2021 August; 30(4):381-400. doi: 10.1007/s11248-021-00252-z; Chennakesavulu et al., Plant Cell Rep. 2022 March; 41(3):815-831. doi: 10.1007/s00299-021-02681-w; and Zhang et al., Nat Plants. 2019 August; 5(8):778-794. doi: 10.1038/s41477-019-0461-5, which can be adapted for use with the present disclosure, particularly to generate the engineered plants having modified epigenomes as described herein. In view of at least the reference sequences in the context of the disclosure herein, one of ordinary skill in the art can apply any one or more of these methods and techniques to generate engineered plants having one or more modified epigenome at or local to ITPK and/or VIP genes and/or encoding polynucleotides. In some embodiments, such as when the engineered plant is generated using a CRISPR-Cas system or CRISPR-based system, the engineered plant comprises a CRISPR-Cas system or component(s) thereof.

Engineered Plants Having Modified ITPK and/or VIP Gene Products

As previously described in some embodiments, the engineered plant has increased amount of one or more ITPK and/or VIP gene products as compared to a suitable control. In some embodiments, the one or more ITPK and/or VIP gene products is mRNA, a polypeptide, or both. In some embodiments, the ITPK and/or VIP gene product is modified as compared to a suitable control. In some embodiments, the suitable control is the unmodified ITPK and/or VIP gene product.

In some embodiments, the ITPK and/or VIP gene product is (a) a modified ITPK and/or VIP mRNA, wherein the mRNA has been modified as compared to an unmodified control to increase protein translation, mRNA stability, or both; (b) a modified ITPK and/or VIP polypeptide, wherein the polypeptide has been modified as compared to an unmodified control to increase protein stability; or both (a) and (b).

mRNA Modifications

In some embodiments, the modified ITPK and/or VIP mRNA comprises (a) one or more mutations, insertions, deletions, substitutions, or any combination thereof such that translation and/or stability of the ITPK and/or VIP mRNA is increased; (b) one or more nucleic acid modifications that increases translation and/or mRNA stability; or both (a) and (b).

In some embodiments, the modifications are genetically encoded in DNA that encodes the mRNA such that when transcribed, the one or more modifications are present in the transcribed mRNA so as to produce the modified mRNA. In other embodiments, the mRNA can be directly modified, such as by a programmable nuclease. Methods of using programmable nuclease systems, such as CRISPR-Cas based systems, and other systems to modify RNA are generally known in the art. See e.g., Porto et al., Nat Rev Drug Discov. 2020 December; 19(12):839-859. doi: 10.1038/s41573-020-0084-6; Rees et al., Nat Rev Genet. 2018 December; 19(12):770-788. doi: 10.1038/s41576-018-0059-1; Xu et al., Mol Cell. 2022 Jan. 20; 82(2):389-403. doi: 10.1016/j.molcel.2021.10.010; Li et al., J Zhejiang Univ Sci B. 2021 Apr. 15; 22(4):253-284. doi: 10.1631/jzus.B2100009; Zeballos and Gaj. Trends Biotechnol. 2021 July; 39(7):692-705. doi: 10.1016/j.tibtech.2020.10.010; Lo et al., Front Genet. 2022 Jan. 28; 13:834413. doi: 10.3389/fgene.2022.834413. eCollection 2022; Li et al., Funct Integr Genomics. 2022 December; 22(6):1089-1103. doi: 10.1007/s10142-022-00910-3; Khosravi and Jantsch. RNA Biol. 2021 Oct. 15; 18(sup1):41-50. doi: 10.1080/15476286.2021.1983288; Kavuri et al., Cells. 2022 Aug. 27; 11(17):2665. doi: 10.3390/cells11172665, which can be adapted for use with the present disclosure, particularly to generate the engineered plants having modified ITPK and/or VIP mRNA.

In some embodiments, an insertion, deletion, or indel in a modified endogenous ITPK and/or VIP gene can range in size from 1-50 or more base pairs. In some embodiments, an insertion, deletion, or indel in a modified endogenous ITPK and/or VIP gene can be 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more.

In some embodiments, the modified ITPK and/or VIP RNA has 1-500 or more mutated or substituted bases or base pairs In some embodiments, the modified ITPK and/or VIP RNA has 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 or more mutated or substituted bases or base pairs.

In some embodiments, the ITPK and/or VIP RNA, such as mRNA contains one or more modifications that can modulate stability of the ITPK and/or VIP RNA. Without being bound by theory some embodiments, where the modification increases stability the translation of an ITPK and/or VIP RNA, such as an mRNA, is increased, which can increase the amount of an ITPK and/or VIP polypeptide. Without being bound by theory some embodiments, where the modification decreases stability the translation of an ITPK and/or VIP RNA, such as an mRNA, is decreased, which can decrease the amount of an ITPK and/or VIP polypeptide. In some embodiments, the modifications that modulate the stability of the ITPK and/or VIP RNA are genetically encoded or otherwise introduced into the sequence of the mRNA. In some embodiments, the modifications that modulate the stability of the ITPK and/or VIP RNA are chemical modifications or synthetic bases that can be coupled to or otherwise integrated into the ITPK and/or VIP RNA.

In some embodiments, the polynucleotides of an ITPK and/or VIP RNA are structurally modified and/or chemically modified. As used in this context herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, the ITPK and/or VIP RNA polynucleotide, e.g., an mRNA, described herein comprises at least one chemical modification. In some embodiments, the at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

In some embodiments, about 10%, 15%, 20%, 24%, 30%, 35%, 40%, 45%, 50%, 55%, 60%, 65%, 70%, 75%, 80%, 85%, 90%, 95%, to/or about 100% of the uracil in of the ITPK and/or VIP RNA, such in the open reading frame of the ITPK and/or VIP encoding polynucleotide, have a chemical modification.

In some embodiments, the ITPK and/or VIP mRNA polynucleotide includes a stabilization element. In some embodiments, the stabilization element is a histone stem-loop. In some embodiments, the stabilization element is a nucleic acid sequence having increased GC content relative to wild type sequence.

In some embodiments, the ITPK and/or VIP mRNA comprises and/or encodes one or more 5′terminal cap (or cap structure), 3′terminal cap, 5′untranslated region, 3′untranslated region, a tailing region, or any combination thereof. In some embodiments, the capping region of the ITPK and/or VIP mRNA region may be from 1 to 10, e.g., 2-9, 3-8, 4-7, 1-5, 5-10, or at least 2, or 10 or fewer nucleotides in length. In some embodiments, the cap is absent. In some embodiments, a 5′-cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine. In some embodiments, the 5′terminal cap is 7mG(5′)ppp(5′)NlmpNp, m7GpppG cap, N′-methylguanine. In some embodiments, the 3′terminal cap is a 3′-O-methyl-m7GpppG.

In some embodiments, the 3′-UTR is an alpha-globin 3′-UTR. In some embodiments, the 5′-UTR comprises a Kozak sequence.

In some embodiments, the tailing sequence may range from absent to 500 nucleotides in length (e.g., at least 60, 70, 80, 90, 120, 140, 160, 180, 200, 250, 300, 350, 400, 450, or 500 nucleotides). In some embodiments, the tailing region is or includes a polyA tail. Where the tailing region is a poly A tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. In some embodiments, the poly-A tail is at least 160 nucleotides in length.

Other RNA modifications for mRNAs and production of mRNA can be as described e.g., U.S. Pat. Nos. 8,278,036, 8,691,966, 8,748,089, 9,750,824, 10,232,055, 10,703,789, 10,702,600, 10,577,403, 10,442,756, 10,266,485, 10,064,959, 9,868,692, 10,064,959, 10,272,150; U.S. Publications, US20130197068, US20170043037, US20130261172, US20200030460, US20150038558, US20190274968, US20180303925, US20200276300; International Patent Application Publication Nos. WO/2018/081638A1, WO/2016/176330A1, which are incorporated herein by reference and can be adapted for use with the present invention.

### Polypeptide Modifications

In some embodiments, the one or more modified gene products is one or more modified ITPK and/or VIP polypeptide. In some embodiments, at least one of the one or more modified ITPK and/or VIP polypeptides has increased activity as compared to an unmodified control. In some embodiments, the engineered plant having increased ITPK and/or VIP enzyme activity has one or more modified ITPK and/or VIP polypeptides. In some embodiments, one or more modified ITPK and/or VIP polypeptides comprises one or more amino acid mutations, insertions, deletions, substitutions, or any combination thereof such that activity of the one or more modified ITPK and/or VIP polypeptides is increased as compared to a control; (b) comprises one or more post-translational modifications such that activity of the one or more modified ITPK and/or VIP polypeptides is increased as compared to a control.

The modified ITPK and/or VIP polypeptide contains 1 or more mutations, insertions, deletions, indels, substitutions, or any combination thereof of one or more continuous or discontinuous amino acids. In some embodiments, an N- and/or C-terminal region is truncated by 1-50 or more amino acids.

In some embodiments, the modification is a post-translational modification. The post translational modification can be reversible or irreversible. The post translational modifications can modulate one or more activities or functions of the ITPK and/or VIP polypeptide, such as protein lifespan, protein-protein interactions, cell to cell and/or cell to cell-matrix interactions, molecular trafficking, receptor activation and/or ligand binding, protein solubility, protein folding, protein localization and/or any combination thereof. Exemplary post translational modifications include, but are not limited to glycosylation, methylation, phosphorylation, acetylation, succinylation, malonylation, sumolation, S-nitrosylation, glutathionylation, amidation, hydroxylation, palmitolation, pyrrolidone carboxcylic acid, glutarylation, gamma-carboxyglutamic acid, crotonylation, oxidation, myristoylation, sulfantion, formylation, and citrullination. See e.g., Ramazi and Zahiri. Database, Volume 2021, 2021, baab012, https://doi.org/10.1093/database/baab012 for additional exemplary post translational modifications. In some embodiments, the amino acid sequence of an ITPK and/or VIP polypeptide is modified to influence the post-translational modification. For example, in some embodiments, the ITPK and/or VIP polypeptide is modified so as to increase or decrease the number of residues capable of being post translationally modified by any one or more post translational modifications.

In some embodiments, the ITPK and/or VIP polypeptide contains one or more signaling and/or trafficking polypeptides. Exemplary signaling and trafficking sequences can be genetically encoded and are described in greater detail elsewhere herein. In some embodiments, the ITPK and/or VIP polypeptide contains or is otherwise operatively coupled to one or more reporter polypeptides and/or polynucleotides. Exemplary reporter polypeptides and/or polynucleotides are described in greater detail elsewhere herein.

### Exemplary Engineered Plants

Also described herein are engineered plants that can contain one or more of the engineered cells expressing an I ITPK and/or VIP gene and/or gene product described herein. In some embodiments, the engineered plant is an engineered monocotyledonous plant or an engineered dicotyledonous plant. In some embodiments the engineered dicotyledonous plant belongs to the order Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Brassicales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, Brassicales, or Asterales.

In some embodiments, the engineered monocotyledonous plant belongs to the order Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, Orchidales, Pinales, Ginkogoales, Cycadales, Araucariales, Cupressales or Gnetales.

In some embodiments, the engineered plant is a species of Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Thlaspi, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, or Pseudotsuga.

In some embodiments, the engineered plant is a fern. In some embodiments, the engineered plant is a moss. In some embodiments, the engineered plant is a liverwort. In some embodiments, the engineered plant is of the phyla Bryophyta, Pterophyta, or Lycopodiophita. In some embodiments, the engineered plant is from the order Sphagnales, Jungermanniales, Marchantiles, Polytrichales, Hypnobryales, Dicranales, Polypodiales, Equisetales, or Selaginellales. In some embodiments, the engineered plant is from the genus Sphagnum, Bazzania, Marchantia, Conocephalum, Riccia, Polytrichum, Pleurozium, Dicranum, Nephrolepis, Equistum, or Selaginella. In some embodiments, the engineered plant is Marchantia polymorpha.

In some embodiments, the engineered plant is a grain crop plant (e.g., wheat, maize, rice, millet, barley), a fruit crop plant (e.g., tomato, apple, pear, strawberry, orange), forage crops (e.g., alfalfa), a root vegetable crop plant (e.g., carrot, potato, sugar beets, yam), a leafy vegetable crop plant (e.g., lettuce, spinach); a flowering crop plant (e.g., petunia, rose, chrysanthemum), a conifers or pine tree (e.g., pine fir, spruce); a plant used in phytoremediation (e.g., heavy metal accumulating plants); an oil crop plant (e.g., sunflower, rape seed), or a plant typically used for experimental purposes (e.g., Arabidopsis).

In some embodiments, the engineered plant is an angiosperm or a gymnosperm plant.

In some embodiments, the engineered plant is acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pennycress, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, or zucchini.

In some embodiments, the engineered plant is a turfgrass. In some embodiments, the engineered plant is of the order Cyperales. In some embodiments, the engineered plant is from the family of Poaceae. In some embodiments, the engineered plant is of the genus Achnatherum P. Beauv. (needlegrass), Achnella Barkworth (ricegrass), Acrachne Chiov. (goosegrass), Acroceras Stapf (Acroceras), Aegilops L. (goatgrass), Aegopogon Humb. & Bonpl. ex Willd. (relaxgrass), Aeluropus Trin. (Indian walnut), ×Agroelymus E.G. camus ex A. camus (Agroelymus), ×Agropogon Fourn. (Agropogon), Agropyron Gaertn. (wheatgrass), Agrostis L. (bentgrass), Aira L. (hairgrass), Allolepis Söderst. & Decker (Texas salt), Alloteropsis J. Presl (summergrass), Alopecurus L. (foxtail), Amblyopyrum Eig (Amblyopyrum), ×Ammocalamagrostis P. Fourn., Ammophila Host (beachgrass), Ampelodesmos Link (Mauritanian grass), Amphibromus Nees (wallaby grass), Amphicarpum Kunth (maidencane), Ancistrachne S.T. Blake, Andropogon L. (bluestem), Anthaenantia P. Beauv. (silkyscale), Anthephora Schreb. (oldfield grass), Anthoxanthum L. (hornwort), Apera Adans. (silkybent), Apluda L. (Mauritian grass), Arctagrostis Griseb. (polargrass), ×Arctodupontia Tzvelev (Arctodupontia), Arctophila Rupr. ex Andersson (pendantgrass), Genus Aristida L. (threeawn), Arrhenatherum P. Beauv. (oatgrass), Arthraxon P. Beauv. (carpetgrass), Arthrostylidium Rupr. (climbing bamboo), Arundinaria Michx. (cane), Arundinella Raddi (rabo de gato), Arundo L. (giant reed), Astrebla F. Muell. ex Benth., Austrostipa S.W.L. Jacobs & J. Everett, Avellinia Parl., Avena L. (oat), Avenula (Dumort.) Dumort. (oatgrass), Axonopus P. Beauv. (carpetgrass), Bambusa Schreb. (bamboo), Beckmannia Host (sloughgrass), Blepharidachne Hack. (desertgrass), Blepharoneuron Nash (dropseed), Borinda Stapleton (Borinda), Bothriochloa Kuntze (beardgrass), Bouteloua Lag. (grama), Brachiaria (Trin.) Griseb. (signalgrass), Brachyachne Stapf, Brachyelytrum P. Beauv. (shorthusk), Brachypodium P. Beauv. (false brome), Brachypodium distachyon, Briza L. (quakinggrass), Bromidium Nees & Meyen (tropical bent), Bromus L. (brome), Calamagrostis Adans. (reedgrass), ×Calammophila Brand (Calammophila), Calamovilfa (A. Gray) Hack. ex Scribn. & Southworth (sandreed), Calyptochloa C.E. Hubb., Castellia Tineo, Catabrosa P. Beauv. (whorlgrass), Catapodium Link (ferngrass), Cathestecum J. Presl (false grama), Celtica F.M. Vázquez & Barkworth, Cenchrus L. (sandbur), Centotheca Desv., Centropodia (R. Br.) Rchb., Chasmanthium Link (woodoats), Chloris Sw. (windmill grass), Chrysopogon Trin. (false beardgrass), Chusquea Kunth (Chusquea bamboo), Cinna L. (woodreed), Cladoraphis Franch. (bristly lovegrass), Cleistachne Benth., Cleistogenes Keng, Coelorachis Brongn. (jointtail grass), Coix L. (Job's tears), Coleanthus Seidel (mossgrass), Cortaderia Stapf (pampas grass), Corynephorus P. Beauv. (clubawn grass), Cottea Kunth (cotta grass), Crypsis Aiton (pricklegrass), Ctenium Panzer (toothache grass), Cutandia Willk. (Memphisgrass), Cymbopogon Spreng. (lemon grass), Cynodon Rich. (Bermudagrass), Cynosurus L. (dogstail grass), Cyrtococcum Stapf, Dactylis L. (orchardgrass), Dactyloctenium Willd. (crowfoot grass), Danthonia DC. (oatgrass), Danthoniopsis Stapf, Dasyochloa Willd. ex Rydb. (woollygrass), Dasypyrum (Coss. & Durieu) T. Dur. (mosquitograss), Dendrocalamus Nees, Deschampsia P. Beauv. (hairgrass), Desmostachya (Stapf) Stapf, Diarrhena P. Beauv. (beakgrain), Dichanthelium (Hitchc. & Chase) Gould (rosette grass), Dichanthium Willem. (bluestem), Dichelachne Endl. (Plumegrass), Diectomis Kunth (foldedleaf grass), Digitaria Haller (crabgrass), Dimeria R. Br., Dinebra Jacq. (viper grass), Dissanthelium Trin. (Catalina grass), Dissochondrus (Hillebr.) Kuntze (false brittlegrass), Distichlis Raf. (saltgrass), ×Dupoa J. Cay. & S. Darbyshire (dupoa), Dupontia R. Br. (tundragrass), Echinochloa P. Beauv. (cockspur grass), Echinopogon P. Beauv., Ectrosia R. Br. (ectrosia), Ehrharta Thunb. (veldtgrass), Eleusine Gaertn. (goosegrass), Elionurus Humb. & Bonpl. ex Willd. (balsamscale grass), ×Elyhordeum Mansf. ex Zizin & Petrowa (barley), ×Elyleymus Baum (wildrye), Elymus L. (wildrye), Elytrigia Desv., Enneapogon Desv. ex P. Beauv. (feather pappusgrass), Enteropogon Nees (umbrellagrass), Entolasia Stapf (entolasia) Eragrostis von Wolf (lovegrass), Eremochloa Büse (centipede grass), Eremopyrum (Ledeb.) Jaubert & Spach (false wheatgrass), Eriachne R. Br., Eriochloa Kunth (cupgrass), Eriochrysis P. Beauv. (moco de pavo), Erioneuron Nash (woollygrass), Euclasta Franch. (mock bluestem), Eulalia Trin., Eulaliopsis Honda (sabaigrass), Eustachys Desv. (fingergrass), Festuca L. (fescue), ×Festulolium Asch. & Graebn. (Festulolium), Fingerhuthia Nees (Zulu fescue), Garnotia Brongn. (lawngrass), Gastridium P. Beauv. (nit grass), Gaudinia P. Beauv. (fragile oat), Gigantochloa Kurz ex Munro (gigantochloa), Glyceria R. Br. (mannagrass), Gymnopogon P. Beauv. (skeletongrass), Gynerium Willd. ex P. Beauv. (wildcane), Hackelochloa Kuntze (pitscale grass), Hainardia Greuter (barbgrass), Hakonechloa Makino ex Honda (Hakone grass), Helictotrichon Besser ex Schult. & Schult. f. (alpine oatgrass), Hemarthria R. Br. (jointgrass), Hesperostipa (Elias) Barkworth (needle and thread), Heteranthelium Hochst. ex Jaub. & Spach, Heteropogon Pers. (tanglehead), Hierochloe R. Br. (sweetgrass), Hilaria Kunth (curly-mesquite), Holcus L. (velvetgrass), Homolepis Chase (panicgrass), Homopholis C.E. Hubb., Hordelymus (Jess.) Harz, Hordeum L. (barley), Hygroryza Nees (watergrass), Hymenachne P. Beauv. (marsh grass), Hyparrhenia Andersson ex Fourn. (thatching grass), Hypogynium Nees (West Indian bluestem), Ichnanthus P. Beauv. (bedgrass), Imperata Cirillo (satintail), Isachne R. Br. (bloodgrass), Ischaemum L. (murainagrass), Iseilema Andersson, Ixophorus Schltdl. (Central America grass), Jarava Ruiz & Pav. (rice grass), Kalinia H.L. Bell & Columbus (kalinia grass), Karroochloa Conert & Türpe (South African oatgrass), Koeleria Pers. (Junegrass), Lagurus L. (harestail grass), Lamarckia Moench (goldentop grass), Lasiacis (Griseb.) Hitchc. (smallcane), Lasiurus Boiss., Leersia Sw. (cutgrass), Leptochloa P. Beauv. (sprangletop), Leptochloopsis Yates (limestone grass), Leptocoryphium Nees (lanilla), Lepturus R. Br. (thintail), Leucopoa Griseb. (spike fescue), ×Leydeum Barkworth (hybrid ryegrass), Leymus Hochst. (wildrye), Limnodea L.H. Dewey (Ozark grass), Lithachne P. Beauv. (diente de perro), Loliolum Krecz. & Bobr., Lolium L. (ryegrass), Lophatherum Brongn. (lophatherum), Loudetia Hochst., Luziola Juss. (watergrass), Lycurus Kunth-(wolfstail), Lygeum Loefl. ex L. (lygeum), Melica L. (melicgrass), Melinis P. Beauv. (stinkgrass), Merxmuellera Conert, Mibora Adans. (sandgrass), Microchloa R. Br. (smallgrass), Microlaena R. Br. (weeping grass), Micropyrum Link, Microstegium Nees (browntop), Milium L. (milletgrass), Miscanthus Andersson (silvergrass), Mnesithea Kunth (jointtail grass), Molinia Schrank (moorgrass), Monanthochloe Engelm. (shoregrass), Muhlenbergia Schreb. (muhly), Munroa Torr., orth. cons. (false buffalograss), Nardus L. (matgrass), Nassella (Trin.) Desv. (needlegrass), Neeragrostis Bush (creeping lovegrass), Neololeba Widjaja (neololeba), Neostapfia Burtt Davy (Colusagrass), Neyraudia Hook. f. (neyraudia), Ochthochloa Edgew., Olyra L. (carrycillo), Ophiuros C.F. Gaertn., Oplismenus P. Beauv. (basketgrass), Orcuttia Vasey (Orcutt grass), Oryza L. (rice), Oryzopsis Michx. (ricegrass), Ottochloa Dandy, Oxychloris M. Lazarides, Oxytenanthera Munro (oxytenanthera), Panicum L. (panicgrass), Pappophorum Schreb. (pappusgrass), Parapholis C.E. Hubbard (sicklegrass), Pascopyrum A. Löve (wheatgrass), Paspalidium Stapf (watercrown grass), Paspalum L. (crowngrass), Patis Ohwi (ricegrass), Pennisetum Rich. ex Pers. (fountaingrass), Perotis Aiton, Phalaris L. (canarygrass), Phanopyrum (Raf.) Nash (savannah-panicgrass), Pharus L. (stalkgrass), Phippsia (Trin.) R. Br. (icegrass), Phleum L. (timothy), Phragmites Adans. (reed), Phyllostachys Siebold & Zucc. (bamboo), Piptatheropsis Romasch., P.M. Peterson & R. J. Soreng (ricegrass), Piptatherum P. Beauv. (ricegrass), Piptochaetium J. Presl (speargrass), Plectrachne Henrard, Pleioblastus Nakai (dwarf bamboo), Pleuraphis Torr. (galleta grass), Pleuropogon R. Br. (semaphoregrass), Poa L. (bluegrass), Pogonatherum P. Beauv., Polypogon Desf. (rabbitsfoot grass), Polytoca R. Br., Polytrias Hack. (Java grass), Psathyrostachys Nevski (wildrye), ×Pseudelymus Barkworth & D.R. Dewey (foxtail wheatgrass), Pseudoroegneria (Nevski) A. Löve (wheatgrass), Pseudosasa Makino ex Nakai (arrow bamboo), Psilurus Trin., Ptilagrostis Griseb. (false needlegrass), Puccinellia Parl. (alkaligrass), ×Pucciphippsia Tzvelev (pucciphippsia), Redfieldia Vasey (blowout grass), Reimarochloa Hitchc. (reimar grass), Rostraria Trin. (hairgrass), Rottboellia L. f. (itchgrass), Rytidosperma Steud. (wallaby grass), Saccharum L. (sugarcane), Sacciolepis Nash (cupscale grass), Sasa Makino & Shib. (broadleaf bamboo), ×Schedolium Holub (fescue ryegrass), Schedonnardus Steud. (tumblegrass), Schedonorus P. Beauv. (fescue), Schismus P. Beauv. (Mediterranean grass), Schizachne Hack. (false melic), Schizachyrium Nees (little bluestem), Schizostachyum Nees (Polynesian 'ohe P), Schmidtia Moench, Sclerochloa P. Beauv. (hardgrass), Scleropogon Phil. (burrograss), Sclerostachya A. camus, Scolochloa Link (rivergrass), Scribneria Hack. (Scribner's grass), Secale L. (rye), Sehima Forssk., Genus Semiarundinaria Makino, Sesleria Scop., Setaria P. Beauv. (bristlegrass), Setaria viridis (e.g. of the Andropogoneae tribe), Setariopsis Scribn. ex Millsp. (setariopsis), Shibataea Makino ex Nakai, Sinocalamus McClure (wideleaf bamboo), Snowdenia C.E. Hubb., Sorghastrum Nash (Indiangrass), Sorghum Moench (sorghum), Spartina Schreb. (cordgrass), Sphenopholis Scribn. (wedgescale), Spinifex L., Spodiopogon Trin., Sporobolus R. Br. (dropseed), Steinchisma Raf. (gaping grass), Stenotaphrum Trin. (St. Augustine grass), Stipa L., Stipagrostis Nees, Swallenia Söderst. & Decker (dunegrass), Taeniatherum Nevski (medusahead), Tetrachne Nees, Tetrapogon Desf., Thaumastochloa C.E. Hubb., Themeda Forssk. (kangaroo grass), Thinopyrum A. Löve (wheatgrass), Thuarea Pers. (Kuroiwa grass), Thyridolepis S.T. Blake, Thysanolaena Nees (tiger grass), Torreyochloa Church (false mannagrass), Trachypogon Nees (crinkleawn grass), Tragus Haller (bur grass), Tribolium Desv. (tribolium), Trichloris Fourn. ex Benth. (false Rhodes grass), Tricholaena Schrad., Trichoneura Andersson (Silveus' grass), Tridens Roem. & Schult. (tridens), Triodia R. Br., Triplasis P. Beauv. (sandgrass), Tripogon Roem. & Schult. (fiveminute grass), Tripsacum L. (gamagrass), Triraphis R. Br. (needlegrass), Trisetum Pers. (oatgrass), ×Triticosecale Wittm. ex A. camus (triticale), Triticum L. (wheat), Tuctoria J. Reeder (spiralgrass), Uniola L. (seaoats), Urochloa P. Beauv. (signalgrass), Vahlodea Fr. (hairgrass), Vaseyochloa Hitchc. (Texasgrass), Ventenata Koeler (North Africa grass), Vetiveria Bory (vetivergrass), Vossia Wall. & Griffith (hippo grass), Vulpia C.C. Gmel. (fescue), Whiteochloa C.E. Hubb., Willkommia Hack. (willkommia), Zea L. (corn), Zingeria P.A. Smirn., Zizania L. (wildrice), Zizaniopsis Döll & Asch. (cutgrass), P. australis, or Zoysia Willd. (lawngrass).

In some embodiments, the engineered plant is of the genus Striga. In some embodiments, the engineered plant is witchweed (e.g., Striga hermonthica).

In some embodiments, the engineered plant is of the genus Cardamine. In some embodiments, the engineered plant is Cardamine hirsute (hairy bittercress).

In some embodiments, the engineered plant is an algae.

In some embodiments, the engineered plant is an algae from the phyla Rhodophyta (red algae), Chlorophyta (green algae), Phaeophyta (brown algae), Bacillariophyta (diatoms), Eustigmatophyta, a dinoflagellates, or the prokaryotic phylum Cyanobacteria (blue-green algae).

In some embodiments, the engineered algae the species of amphora, Anabaena, Anikstrodesmis, Botryococcus, Chaetoceros, Chlamydomonas, Chlorella, chlorococcum, cyclotella, Cylindrotheca, Dunaliella, Emiliana, euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, or Trichodesmium.

In some embodiments, the engineered cell, the engineered plant, or both include an ITPK and/or VIP gene and/or ITPK and/or VIP gene product encoding polynucleotide stably integrated into the genome of the engineered cell.

In some embodiments, the engineered cell, the engineered plant, or both include a ITPK and/or VIP gene and/or ITPK and/or VIP gene product encoding polynucleotide that is transiently expressed in the engineered cell, engineered plant, or both.

Methods of Generating ITPK, VIP, and/or PSR Overexpressing Plants

Also described herein are methods of modifying a plant cell such that it contains and optionally expresses an ITPK and/or VIP polypeptide and/or ITPK and/or VIP gene and/or ITPK and/or VIP gene product encoding polynucleotide and/or vector or vector system containing an ITPK and/or VIP gene product encoding polynucleotide, or a combination thereof. In some embodiments, the method includes modifying a plant cell such that the plant cell contains and optionally expresses a heterologous or non-native ITPK and/or VIP R polypeptide, a vector or vector system containing a non-native or heterologous ITPK and/or VIP gene or ITPK and/or VIP gene product encoding polynucleotide, or a combination thereof. In some embodiments, modifying includes delivering a polynucleotide (a) having a sequence that is about 70-100% (e.g., 70 to/or 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%) identical to any one of SEQ ID NO: 1-2 or 5-6; (b) that encodes a polypeptide that has a sequence that is about 70-100% (e.g., 70 to/or 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%) identical to any one of SEQ ID NO: 3-4 or 7-8; or (c) both (a) and (b); or a vector or vector system thereof to the cell; delivering a polynucleotide (a) having a sequence that is about 70-100% (e.g., 70 to/or 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%) identical to any one of SEQ ID NO: 1-2 or 5-6; (b) that encodes a polypeptide that has a sequence that is about 70-100% (e.g., 70 to/or 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%) identical to any one of SEQ ID NO: 3-4 or 7-8; or (c) both (a) and (b) to the cell; or both.

In some embodiments, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

“Plant” as used herein encompasses any plant tissue or part of the plant of the invention. In some embodiments, said part is selected from the group of a plant cell, a somatic embryo, a pollen, a gametophyte, an ovule, an inflorescence, a leaf, a seedling, a stem, a callus, a stolon, a microtuber, a root, a shoot, a seed, a fruit and a spore. Further encompassed are T1 generation plants produced from the seeds of the transformed plant (TO). Any suitable method can be used to confirm and detect the modification made in the plant. Such methods are generally known in the art. In some examples, when a variety of modifications are made, one or more desired modifications or traits resulting from the modifications may be selected and detected. The detection and confirmation may be performed by biochemical and molecular biology techniques such as Southern analysis, PCR, Northern blot, S1 RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

A part of a plant, e.g., a “plant tissue” can be engineered to include an ITPK and/or VIP gene product encoding polynucleotide, vector, and/or polypeptide described elsewhere herein to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized unites, such as, for example, plant tissue, a plant organ, or a whole plant.

A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processing colored substances (e.g., the β-glucuronidase, luciferase, B or C1 genes).

Any suitable method may be used to deliver the transgene to the plant, plant cell, and/or plant cell population. Such transformation techniques are generally known in the art. Example methods and techniques include those in U.S. Pat. No. 6,603,061—Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, Morrell et al “Crop genomics: advances and applications,” Nat Rev Genet. 2011 Dec. 29; 13(2):85-96, all the contents and disclosure of each of which are herein incorporated by reference in their entirety.

In some embodiments, where transient expression is desired transgene DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

A method of generating engineered cells and/or plants can include transformation of one or more cells. The term “transformation” broadly refers to the process by which a plant host is genetically modified by the introduction of DNA by means of Agrobacteria or one of a variety of chemical or physical methods. As used herein, the term “plant host” refers to plants, including any cells, tissues, organs, or progeny of the plants. Many suitable plant tissues or plant cells can be transformed and include, but are not limited to, protoplasts, somatic embryos, pollen, leaves, seedlings, stems, calli, stolons, microtubers, roots, and shoots. A plant tissue also refers to any clone of such a plant, seed, progeny, propagule whether generated sexually or asexually, and descendants of any of these, such as cuttings or seed. The term “transformed” as used herein, refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is transmitted to the subsequent progeny. In these embodiments, the “transformed” or “transgenic” cell or plant may also include progeny of the cell or plant and progeny produced from a breeding program employing such a transformed plant as a parent in a cross and exhibiting an altered phenotype resulting from the presence of the introduced DNA molecule. Preferably, the transgenic plant is fertile and capable of transmitting the introduced DNA to progeny through sexual reproduction.

In some embodiments, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the ITPK and/or VIP transgene or other ITPK and/or VIP genetic or epigenetic modification system is expressed. Vectors and vector systems are described in greater detail elsewhere herein.

The term plant also encompasses progeny of the plant. The term “progeny”, such as the progeny of a transgenic (or engineered) plant, is one that is born of, begotten by, or derived from a plant or the transgenic plant. The introduced DNA molecule may also be transiently introduced into the recipient cell such that the introduced DNA molecule is not inherited by subsequent progeny and thus not considered “transgenic”. Accordingly, as used herein, a “non-transgenic” plant or plant cell is a plant which does not contain a foreign DNA stably integrated into its genome.

Also described herein are gametes, seeds, germplasm, embryos, either zygotic or somatic, progeny or hybrids of plants comprising the genetic modification (e.g., inclusion and/or expression of an ITPK and/or VIP encoding polynucleotide or modified ITPK and/or VIP polynucleotide), which are produced by traditional breeding methods, are also included within the scope of the present invention. Such plants may contain a heterologous or foreign DNA sequence inserted at or instead of a target sequence. Alternatively, such plants may contain only an alteration (mutation, deletion, insertion, substitution) in one or more nucleotides. As such, such plants will only be different from their progenitor plants by the presence of the particular modification.

### Stable Integration in the Genome of Plants and Plant Cells

In particular embodiments, the polynucleotides encoding ITPK and/or VIP gene product are introduced for stable integration into the genome of a plant cell. In these embodiments, the design of the transformation vector or the expression system can be adjusted depending on for when, where and under what conditions the ITPK and/or VIP gene product encoding polynucleotides are expressed. Suitable vectors and delivery are described in greater detail elsewhere herein.

In particular embodiments, the ITPK and/or VIP gene product encoding polynucleotides are stably introduced into the genomic DNA of a plant cell. In particular embodiments, the ITPK and/or VIP gene product encoding polynucleotides are introduced for stable integration into the DNA of a plant organelle such as, but not limited to a plastid, mitochondrion or a chloroplast. In some embodiments, the expression system for stable integration into the genome of a plant cell can contain one or more of the following elements: a promoter element that can be used to express ITPK and/or VIP gene product encoding polynucleotide(s) in a plant cell; a 5′ untranslated region to enhance expression; an intron element to further enhance expression in certain cells, such as monocot cells; a multiple-cloning site to provide convenient restriction sites for inserting the polynucleotide modifying agent(s) or a system thereof and other desired elements; and a 3′ untranslated region to provide for efficient termination of the expressed transcript. The elements of the expression system may be on one or more expression constructs which are either circular such as a plasmid or transformation vector, or non-circular such as linear double stranded DNA.

DNA construct(s) containing the components of the systems, and, where applicable, template sequence may be introduced into the genome of a plant, plant part, or plant cell by a variety of conventional techniques. The process generally comprises the steps of selecting a suitable host cell or host tissue, introducing the construct(s) into the host cell or host tissue.

In particular embodiments, the DNA construct may be introduced into the plant cell using techniques such as but not limited to electroporation, microinjection, aerosol beam injection of plant cell protoplasts, or the DNA constructs can be introduced directly to plant tissue using biolistic methods, such as DNA particle bombardment (see also Fu et al., Transgenic Res. 2000 February; 9(1):11-9). The basis of particle bombardment is the acceleration of particles coated with gene/s of interest toward cells, resulting in the penetration of the protoplasm by the particles and typically stable integration into the genome. (see, e.g., Klein et al, Nature (1987), Klein et ah, Bio/Technology (1992), Casas et ah, Proc. Natl. Acad. Sci. USA (1993).).

In particular embodiments, the DNA constructs containing components of the systems may be introduced into the plant by Agrobacterium-mediated transformation. The DNA constructs may be combined with suitable T-DNA flanking regions and introduced into a conventional Agrobacterium tumefaciens host vector. The foreign DNA can be incorporated into the genome of plants by infecting the plants or by incubating plant protoplasts with Agrobacterium bacteria, containing one or more Ti (tumor-inducing) plasmids. (see, e.g., Fraley et al., (1985), Rogers et al., (1987) and U.S. Pat. No. 5,563,055).

### Transient Expression of in Plants and Plant Cells

In some embodiments, the ITPK and/or VIP gene product encoding polynucleotides can be transiently expressed in the plant cell. In these embodiments, the system can ensure ITPK and/or VIP gene product expression and any Pi accumulation can further be controlled. As the expression of the necessary components of the ITPK and/or VIP gene product encoding polynucleotide(s) is transient, plants regenerated from such plant cells typically contain no foreign DNA.

In particular embodiments, the ITPK and/or VIP gene product encoding polynucleotides can be transiently introduced in the plant cells using a plant viral vector (Scholthof et al. 1996, Annu Rev Phytopathol. 1996; 34:299-323). In further particular embodiments, said viral vector is a vector from a DNA virus. For example, geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). In other particular embodiments, said viral vector is a vector from an RNA virus. For example, tobravirus (e.g., tobacco rattle virus, tobacco mosaic virus), potexvirus (e.g., potato virus X), or hordeivirus (e.g., barley stripe mosaic virus). The replicating genomes of plant viruses are non-integrative vectors. Other suitable vectors are described elsewhere herein.

In particular embodiments, the vector used for transient expression of constructs in plants is for instance a pEAQ vector, which is tailored for Agrobacterium-mediated transient expression (Sainsbury F. et al., Plant Biotechnol J. 2009 September; 7(7):682-93) in the protoplast. Precise targeting of genomic locations was demonstrated using a modified Cabbage Leaf Curl virus (CaLCuV) vector to express gRNAs in stable transgenic plants expressing a CRISPR enzyme (Scientific Reports 5, Article number: 14926 (2015), doi: 10.1038/srep14926).

In particular embodiments, double-stranded DNA fragments encoding ITPK and/or VIP gene product can be transiently introduced into the plant cell. In such embodiments, the introduced double-stranded DNA fragments are provided in sufficient quantity to modify the cell but do not persist after a contemplated period of time has passed or after one or more cell divisions. Methods for direct DNA transfer in plants are known by the skilled artisan (see for instance Davey et al. Plant Mol Biol. 1989 September; 13(3):273-85.)

In other embodiments, an RNA polynucleotide encoding the ITPK and/or VIP gene product is introduced into the plant cell, which is then translated and processed by the host cell generating the protein in sufficient quantity accumulate Pi but which does not persist after a contemplated period of time has passed or after one or more cell divisions. Methods for introducing mRNA to plant protoplasts for transient expression are known by the skilled artisan (see for instance in Gallie, Plant Cell Reports (1993), 13; 119-122).

In some embodiments, a combination of the different methods described above can be used.

Translocation to and/or Expression in Specific Plant Organelles

The system may comprise elements for translocation to and/or expression in a specific plant organelle. In some embodiments, a tissue specific promoter can be included in the expression construct. In some embodiments, a tissue localization or organelle localization sequence or signal can be incorporated into the expression constructs. Such promoters and localization signals are described in greater detail elsewhere herein and/or will be appreciated by one of ordinary skill in the art.

### Chloroplast Targeting

In some embodiments, the engineered plants can be engineered to contain modified chloroplast genes or to ensure expression in the chloroplast. In some embodiments, chloroplast transformation methods or compartmentalization of the ITPK and/or VIP gene product encoding polynucleotides and/or polypeptides to the chloroplast. For instance, the introduction of genetic modifications in the plastid genome can reduce biosafety issues such as gene flow through pollen.

Methods of chloroplast transformation are known in the art and include Particle bombardment, PEG treatment, and microinjection. Additionally, methods involving the translocation of transformation cassettes from the nuclear genome to the plastid can be used as described in WO2010061186.

In some embodiments, one or more of the ITPK and/or VIP gene product encoding polynucleotides can be targeted to the plant chloroplast. This can be achieved by incorporating in the expression construct a sequence encoding a chloroplast transit peptide (CTP) or plastid transit peptide, operably linked to the 5′ region of the sequence encoding the Cas protein. The CTP is removed in a processing step during translocation into the chloroplast. Chloroplast targeting of expressed proteins is well known to the skilled artisan (see for instance Protein Transport into Chloroplasts, 2010, Annual Review of Plant Biology, Vol. 61:157-180). In such embodiments it is also can be desirable to target the guide RNA to the plant chloroplast. Methods and constructs which can be used for translocating guide RNA into the chloroplast by means of a chloroplast localization sequence are described, for instance, in US20040142476, incorporated herein by reference. Such variations of constructs can be incorporated into the expression systems of the invention to efficiently translocate the ITPK and/or VIP gene product encoding polynucleotides.

### Methods of Using the Engineered Plants

Also described herein are methods of using the engineered plants of the present invention for improved phosphorous utilization and/or soil remediation. Without being bound by theory, the engineered plants of the present invention can have increased phosphorus use efficiency such that they can grow better in growth mediums that have low amounts of or low availability of phosphorus and/or Pi as compared to a suitable control (e.g., wild-type or unmodified plants). In some embodiments, a method includes planting, growing, harvesting, and/or cultivating one or more engineered plants in a growth medium, wherein the growth medium has low phosphorous amounts, low phosphorus availability, or both. In some embodiments, the growth medium has low Pi, low Pi availability, or both. In some embodiments, the growth medium is a soil or an aqueous environment. In this context herein, “low” refers to an amount of or an availability of P and/or Pi in the growth medium that is less than ideal, optimal, or required for growth and/or development of a suitable control plant to the engineered plant of the present invention. In some embodiments, “low” is less than 1 mM P or Pi. In some embodiments, a low P or Pi amount or availability is less than 1 mM P or Pi but greater than 0 mM P or Pi. In some embodiments, a low P or Pi amount or availability is 0.01 mM up to and including 1 mM P or Pi. In some embodiments, a low P or Pi amount or availability is 0.01 mM up to but excluding 1 mM P or Pi. In some embodiments, a low P or Pi amount or availability is 0.01 mM, to/or 0.02 mM, 0.03 mM, 0.04 mM, 0.05 mM, 0.06 mM, 0.07 mM, 0.08 mM, 0.09 mM, 0.1 mM, 0.11 mM, 0.12 mM, 0.13 mM, 0.14 mM, 0.15 mM, 0.16 mM, 0.17 mM, 0.18 mM, 0.19 mM, 0.2 mM, 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.41 mM, 0.42 mM, 0.43 mM, 0.44 mM, 0.45 mM, 0.46 mM, 0.47 mM, 0.48 mM, 0.49 mM, 0.5 mM, 0.51 mM, 0.52 mM, 0.53 mM, 0.54 mM, 0.55 mM, 0.56 mM, 0.57 mM, 0.58 mM, 0.59 mM, 0.6 mM, 0.61 mM, 0.62 mM, 0.63 mM, 0.64 mM, 0.65 mM, 0.66 mM, 0.67 mM, 0.68 mM, 0.69 mM, 0.7 mM, 0.71 mM, 0.72 mM, 0.73 mM, 0.74 mM, 0.75 mM, 0.76 mM, 0.77 mM, 0.78 mM, 0.79 mM, 0.8 mM, 0.81 mM, 0.82 mM, 0.83 mM, 0.84 mM, 0.85 mM, 0.86 mM, 0.87 mM, 0.88 mM, 0.89 mM, 0.9 mM, 0.91 mM, 0.92 mM, 0.93 mM, 0.94 mM, 0.95 mM, 0.96 mM, 0.97 mM, 0.98 mM, 0.99 mM up to but excluding 1 mM. In some embodiments, a low P or Pi amount or availability is 0.01 mM, to/or 0.02 mM, 0.03 mM, 0.04 mM, 0.05 mM, 0.06 mM, 0.07 mM, 0.08 mM, 0.09 mM, 0.1 mM, 0.11 mM, 0.12 mM, 0.13 mM, 0.14 mM, 0.15 mM, 0.16 mM, 0.17 mM, 0.18 mM, 0.19 mM, 0.2 mM, 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.41 mM, 0.42 mM, 0.43 mM, 0.44 mM, 0.45 mM, 0.46 mM, 0.47 mM, 0.48 mM, 0.49 mM, 0.5 mM, 0.51 mM, 0.52 mM, 0.53 mM, 0.54 mM, 0.55 mM, 0.56 mM, 0.57 mM, 0.58 mM, 0.59 mM, 0.6 mM, 0.61 mM, 0.62 mM, 0.63 mM, 0.64 mM, 0.65 mM, 0.66 mM, 0.67 mM, 0.68 mM, 0.69 mM, 0.7 mM, 0.71 mM, 0.72 mM, 0.73 mM, 0.74 mM, 0.75 mM, 0.76 mM, 0.77 mM, 0.78 mM, 0.79 mM, 0.8 mM, 0.81 mM, 0.82 mM, 0.83 mM, 0.84 mM, 0.85 mM, 0.86 mM, 0.87 mM, 0.88 mM, 0.89 mM, 0.9 mM, 0.91 mM, 0.92 mM, 0.93 mM, 0.94 mM, 0.95 mM, 0.96 mM, 0.97 mM, 0.98 mM, 0.99 mM, 1 mM.

In some embodiments, the engineered plant has one or more ITPK and/or VIP genes or gene product encoding polynucleotides under control of an inducible promoter. In some embodiments, expression of one or more ITPK and/or VIP genes is not induced until after the engineered plant has reached a desired growth stage, until after a fruit or other component of the plant has been harvested, and/or until after a set period of time after planting, germinating, and/or sprouting.

In some embodiments, the ITPK and/or VIP gene and/or gene product overexpression is not induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days post planting, germinating, and/or sprouting. In some embodiments, the ITPK and/or VIP gene and/or gene product overexpression is not induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks post planting, germinating, and/or sprouting. In some embodiments, the ITPK and/or VIP gene and/or gene product overexpression is not induced for 1, 2, 3, 4, 5, or more years post planting, germinating, and/or sprouting.

In some embodiments, the ITPK and/or VIP gene and/or gene product overexpression is induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the ITPK and/or VIP gene and/or gene product overexpression is induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks. In some embodiments, the ITPK and/or VIP gene and/or gene product overexpression is induced for 1, 2, 3, 4, 5, or more years.

As previously described and without being bound by theory, because the engineered plants can grow in conditions with low and/or sub-optimal P and/or Pi availability, the engineered plants can have a lower or eliminated need for supplemental P and/or Pi as compared to a suitable control plant, such as that that can be provided in a fertilizer applied to plants. In some embodiments, the method includes planting, growing, harvesting, and/or cultivating comprises a reduced application of an amount of supplemental phosphorous and/or Pi as compared to planting, growing, harvesting, and/or cultivating a suitable control plant. In some embodiments, the method includes planting, growing, harvesting, and/or cultivating the engineered plants with no or reduced supplemental P and/or Pi (including but not limited to that which could be provided in a fertilizer) as compared to a suitable control plant and/or planting, growing, harvesting, and/or cultivating conditions. In some embodiments, the amount of supplemental P and/or Pi applied during planting, growing, harvesting, and/or cultivating is reduced by 1-1000 fold or more. In some embodiments, the amount of supplemental P and/or Pi applied during planting, growing, harvesting, and/or cultivating is reduced by 0.01-100%, 0.01-100%, or 1-100%, with a 100% reduction meaning that no supplemental P and/or Pi is provided to the engineered plant during planting, growing, harvesting, and/or cultivating.

In some embodiments, the amount of supplemental P and/or Pi applied during planting, growing, harvesting, and/or cultivating is reduced by 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 fold or more.

In some embodiments, the amount of supplemental P and/or Pi applied during planting, growing, harvesting, and/or cultivating is reduced by 0.01%, to/or 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, 30%, 30.1%, 30.2%, 30.3%, 30.4%, 30.5%, 30.6%, 30.7%, 30.8%, 30.9%, 31%, 31.1%, 31.2%, 31.3%, 31.4%, 31.5%, 31.6%, 31.7%, 31.8%, 31.9%, 32%, 32.1%, 32.2%, 32.3%, 32.4%, 32.5%, 32.6%, 32.7%, 32.8%, 32.9%, 33%, 33.1%, 33.2%, 33.3%, 33.4%, 33.5%, 33.6%, 33.7%, 33.8%, 33.9%, 34%, 34.1%, 34.2%, 34.3%, 34.4%, 34.5%, 34.6%, 34.7%, 34.8%, 34.9%, 35%, 35.1%, 35.2%, 35.3%, 35.4%, 35.5%, 35.6%, 35.7%, 35.8%, 35.9%, 36%, 36.1%, 36.2%, 36.3%, 36.4%, 36.5%, 36.6%, 36.7%, 36.8%, 36.9%, 37%, 37.1%, 37.2%, 37.3%, 37.4%, 37.5%, 37.6%, 37.7%, 37.8%, 37.9%, 38%, 38.1%, 38.2%, 38.3%, 38.4%, 38.5%, 38.6%, 38.7%, 38.8%, 38.9%, 39%, 39.1%, 39.2%, 39.3%, 39.4%, 39.5%, 39.6%, 39.7%, 39.8%, 39.9%, 40%, 40.1%, 40.2%, 40.3%, 40.4%, 40.5%, 40.6%, 40.7%, 40.8%, 40.9%, 41%, 41.1%, 41.2%, 41.3%, 41.4%, 41.5%, 41.6%, 41.7%, 41.8%, 41.9%, 42%, 42.1%, 42.2%, 42.3%, 42.4%, 42.5%, 42.6%, 42.7%, 42.8%, 42.9%, 43%, 43.1%, 43.2%, 43.3%, 43.4%, 43.5%, 43.6%, 43.7%, 43.8%, 43.9%, 44%, 44.1%, 44.2%, 44.3%, 44.4%, 44.5%, 44.6%, 44.7%, 44.8%, 44.9%, 45%, 45.1%, 45.2%, 45.3%, 45.4%, 45.5%, 45.6%, 45.7%, 45.8%, 45.9%, 46%, 46.1%, 46.2%, 46.3%, 46.4%, 46.5%, 46.6%, 46.7%, 46.8%, 46.9%, 47%, 47.1%, 47.2%, 47.3%, 47.4%, 47.5%, 47.6%, 47.7%, 47.8%, 47.9%, 48%, 48.1%, 48.2%, 48.3%, 48.4%, 48.5%, 48.6%, 48.7%, 48.8%, 48.9%, 49%, 49.1%, 49.2%, 49.3%, 49.4%, 49.5%, 49.6%, 49.7%, 49.8%, 49.9%, 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9%, 55%, 55.1%, 55.2%, 55.3%, 55.4%, 55.5%, 55.6%, 55.7%, 55.8%, 55.9%, 56%, 56.1%, 56.2%, 56.3%, 56.4%, 56.5%, 56.6%, 56.7%, 56.8%, 56.9%, 57%, 57.1%, 57.2%, 57.3%, 57.4%, 57.5%, 57.6%, 57.7%, 57.8%, 57.9%, 58%, 58.1%, 58.2%, 58.3%, 58.4%, 58.5%, 58.6%, 58.7%, 58.8%, 58.9%, 59%, 59.1%, 59.2%, 59.3%, 59.4%, 59.5%, 59.6%, 59.7%, 59.8%, 59.9%, 60%, 60.1%, 60.2%, 60.3%, 60.4%, 60.5%, 60.6%, 60.7%, 60.8%, 60.9%, 61%, 61.1%, 61.2%, 61.3%, 61.4%, 61.5%, 61.6%, 61.7%, 61.8%, 61.9%, 62%, 62.1%, 62.2%, 62.3%, 62.4%, 62.5%, 62.6%, 62.7%, 62.8%, 62.9%, 63%, 63.1%, 63.2%, 63.3%, 63.4%, 63.5%, 63.6%, 63.7%, 63.8%, 63.9%, 64%, 64.1%, 64.2%, 64.3%, 64.4%, 64.5%, 64.6%, 64.7%, 64.8%, 64.9%, 65%, 65.1%, 65.2%, 65.3%, 65.4%, 65.5%, 65.6%, 65.7%, 65.8%, 65.9%, 66%, 66.1%, 66.2%, 66.3%, 66.4%, 66.5%, 66.6%, 66.7%, 66.8%, 66.9%, 67%, 67.1%, 67.2%, 67.3%, 67.4%, 67.5%, 67.6%, 67.7%, 67.8%, 67.9%, 68%, 68.1%, 68.2%, 68.3%, 68.4%, 68.5%, 68.6%, 68.7%, 68.8%, 68.9%, 69%, 69.1%, 69.2%, 69.3%, 69.4%, 69.5%, 69.6%, 69.7%, 69.8%, 69.9%, 70%, 70.1%, 70.2%, 70.3%, 70.4%, 70.5%, 70.6%, 70.7%, 70.8%, 70.9%, 71%, 71.1%, 71.2%, 71.3%, 71.4%, 71.5%, 71.6%, 71.7%, 71.8%, 71.9%, 72%, 72.1%, 72.2%, 72.3%, 72.4%, 72.5%, 72.6%, 72.7%, 72.8%, 72.9%, 73%, 73.1%, 73.2%, 73.3%, 73.4%, 73.5%, 73.6%, 73.7%, 73.8%, 73.9%, 74%, 74.1%, 74.2%, 74.3%, 74.4%, 74.5%, 74.6%, 74.7%, 74.8%, 74.9%, 75%, 75.1%, 75.2%, 75.3%, 75.4%, 75.5%, 75.6%, 75.7%, 75.8%, 75.9%, 76%, 76.1%, 76.2%, 76.3%, 76.4%, 76.5%, 76.6%, 76.7%, 76.8%, 76.9%, 77%, 77.1%, 77.2%, 77.3%, 77.4%, 77.5%, 77.6%, 77.7%, 77.8%, 77.9%, 78%, 78.1%, 78.2%, 78.3%, 78.4%, 78.5%, 78.6%, 78.7%, 78.8%, 78.9%, 79%, 79.1%, 79.2%, 79.3%, 79.4%, 79.5%, 79.6%, 79.7%, 79.8%, 79.9%, 80%, 80.1%, 80.2%, 80.3%, 80.4%, 80.5%, 80.6%, 80.7%, 80.8%, 80.9%, 81%, 81.1%, 81.2%, 81.3%, 81.4%, 81.5%, 81.6%, 81.7%, 81.8%, 81.9%, 82%, 82.1%, 82.2%, 82.3%, 82.4%, 82.5%, 82.6%, 82.7%, 82.8%, 82.9%, 83%, 83.1%, 83.2%, 83.3%, 83.4%, 83.5%, 83.6%, 83.7%, 83.8%, 83.9%, 84%, 84.1%, 84.2%, 84.3%, 84.4%, 84.5%, 84.6%, 84.7%, 84.8%, 84.9%, 85%, 85.1%, 85.2%, 85.3%, 85.4%, 85.5%, 85.6%, 85.7%, 85.8%, 85.9%, 86%, 86.1%, 86.2%, 86.3%, 86.4%, 86.5%, 86.6%, 86.7%, 86.8%, 86.9%, 87%, 87.1%, 87.2%, 87.3%, 87.4%, 87.5%, 87.6%, 87.7%, 87.8%, 87.9%, 88%, 88.1%, 88.2%, 88.3%, 88.4%, 88.5%, 88.6%, 88.7%, 88.8%, 88.9%, 89%, 89.1%, 89.2%, 89.3%, 89.4%, 89.5%, 89.6%, 89.7%, 89.8%, 89.9%, 90%, 90.1%, 90.2%, 90.3%, 90.4%, 90.5%, 90.6%, 90.7%, 90.8%, 90.9%, 91%, 91.1%, 91.2%, 91.3%, 91.4%, 91.5%, 91.6%, 91.7%, 91.8%, 91.9%, 92%, 92.1%, 92.2%, 92.3%, 92.4%, 92.5%, 92.6%, 92.7%, 92.8%, 92.9%, 93%, 93.1%, 93.2%, 93.3%, 93.4%, 93.5%, 93.6%, 93.7%, 93.8%, 93.9%, 94%, 94.1%, 94.2%, 94.3%, 94.4%, 94.5%, 94.6%, 94.7%, 94.8%, 94.9%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, 100%.

Products Produced from the Engineered Plants

In some embodiments, the engineered plants and/or parts (e.g., fruits, nuts, seeds, grains, etc.) of the present invention are harvested. In some embodiments, one or more parts or the whole plant is harvested for fertilizer. In some embodiments, after one or more parts of the engineered plant of the present invention is harvested for a non-fertilizer product, the rest is harvested for fertilizer production. Once an engineered plant of the present invention has accumulated the Pi, it can be harvested and be used in any suitable form as a fertilizer. In this way, sequestered Pi can be reapplied when desired as a fertilizer and thus serve to recycle phosphorous.

In some embodiments, the harvested engineered plants or parts thereof are applied as a fresh-biomass fertilizer. Such fresh biomass can be chopped, shredded, pulverized, and/or otherwise mechanically broken down prior to application.

In other embodiments, the harvested engineered plants and/or parts thereof of the present invention are further processed, such as carbonized, prior to applying as a fertilizer. In some embodiments, the harvested engineered plants and/or parts thereof of the present invention are processed into biochar. “Biochar” as used herein is a term of art and refers to a charcoal-like byproduct of the process of pyrolysis, or the anaerobic (meaning without oxygen) thermal decomposition of organic material, particularly plant material in the present context. The biochar can be applied as a fertilizer for plant cultivation.

The cycle can be continued by growing engineered plants and/or parts thereof of the present invention in soil fertilized with fresh biomass and/or biochar made from engineered plants and/or parts thereof of the present invention.

In some embodiments, the biochar is combined with one or more other nutrients, soil fortifiers, soils, mulches, binders, or other soil additives.

Methods of Fertilizing Using the Engineered Plants and/or Fertilizers Produced Therefrom

Also described herein is methods of using biochar and/or fresh biomass produced from engineered plants and/or parts thereof of the present invention that have accumulated/sequestered Pi from the soil in which they were grown as a fertilizer. In some embodiments, biochar and/or fresh biomass produced from the engineered plants or parts thereof of the present invention have accumulated/sequestered Pi from the soil in which they were grown is applied to an area in which plants will be grown or are actively growing as a source of phosphorus for growth of the plants.

In some embodiments, biochar and/or fresh biomass is applied 1-10 (e.g., 1, 2, 3, 4, 5, 6, 7, 8, 9, 10) or more times to an area in which plants will be grown or are growing in a year.

The amount of phosphorous and other nutrient qualities can be measured by any suitable methods and/or techniques. Such methods and/or techniques will be appreciated by one of ordinary skill in the art and/or are described elsewhere herein.

The amount of fresh biomass or biochar applied to any given area, will depend on, for example, the plant to which it is being applied, the starting soil nutrient load, the amount allowed as per nutrient management plan and/or local, state, or federal limit imposed on the particular area, the specific Pi present in the biomass and/or biochar being applied, and the like. Other relevant factors will be appreciated by one of ordinary skill in the art in view of this disclosure.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

### Introduction

Phosphorous is a crucial macronutrient necessary for plant growth and development and is a major limiting factor in crop yield in the developed world. Plants uptake phosphate in the form of inorganic phosphate (Pi), however, Pi is often limited in agricultural soils. In addition, Pi is a non-renewable resource with current reserves projected to be depleted in as early as 30 years (Cordell & White 2011; Herrera-Estrella & López-Arredondo 2016). Under low Pi, plants undergo dynamic physiological responses to scavenge Pi from internal sources and increase Pi uptake from their environment (Chien et al. 2018; Chiou & Lin 2011). These responses, known as the Phosphate Starvation Response (PSR), can be identified by phenotypic changes such as root architecture. Under low Pi, plants will prioritize the elongation of lateral roots and production of root hairs to increase surface area and increase Pi uptake (Péret et al. 2011). Other physiological responses include increased anthocyanin production, lipid remodeling, increased production of Pi transporters to increase Pi uptake, and secretion of phosphatases to increase available Pi uptake from the environment (Chien et al. 2018; Nakamura 2013).

Driving the dynamic physiological changes that occur during the PSR is the transcriptional reprogramming that occurs in both the root and shoot under low Pi. This transcriptional reprogramming is controlled mostly by the transcription factors, phosphate starvation response 1 (PHR1) and its paralog, PHR1-like (PHL1) (Bustos et al. 2010; Rubio 2001). PHR1 and PHL1 regulate the transcription of PSR genes through binding to a GNATATNC motif (PHR1 binding sequence; P1BS), found in the promoters of many PSR genes, activating gene expression. PHR1 interacts with stand-alone SPX (Syg1/Pho81/Xpr1) proteins in a Pi-dependent manner, reducing the DNA-binding ability of PHR1 (Puga et al. 2014). Originally, it was thought that Pi facilitates the SPX: PHR1 interaction, however, recent studies have identified inositol phosphates (InsPs) and more so, inositol pyrophosphates (PP-InsPs) as the preferred ligands (Wild et al. 2016).

InsPs are a family of signaling molecules found across eukaryotes derived from a 6-carbon polyol, myo-inositol. InsPs are synthesized by InsP kinases that reversibly add phosphate groups to specific positions on myo-inositol. InsP₆ is the most abundant InsP molecule in plants, serving as the primary phosphate and energy store in many cell types (Raboy 2007). The last step in InsP₆ synthesis involves the conversion of InsP₅ to InsP₆ by inositol pentakisphosphate 2-kinase 1 (IPK1) (Sweetman et al. 2007; Verbsky et al. 2002). PP-InsPs are molecules that contain high-energy phosphoanhydride bonds. InsP₆ can be converted to the PP-InsP, 5-PP-InsP₅, also known as InsP₇, by the multifunctional inositol tetrakisphosphate 1-kinase 1 (ITPK1) (Adepoju et al. 2019; Laha et al. 2019). The diphosphoinositol pentakisphosphate (PPIP5K) enzymes catalyze InsP₈ synthesis from InsP₇, (Adepoju et al. 2019; Desai et al. 2014; Laha et al. 2019; Land et al. 2021; Mulugu et al. 2007; Wang et al. 2012), and also referred to as VIP or VIH.

In plants there are 2 VIP genes, that like their counterparts in other species, encode a pair of dual-domain enzymes consisting of a kinase domain (KD) linked to a C-terminal phosphatase domain (PD) (Gokhale et al. 2011). The KD of VIP1 and VIP2 has been characterized as a 1-kinase, phosphorylating 5-PP-InsP₅ to produce 1,5-PP-InsP₅, the most common form of InsP₈ (Zhu et al. 2019). However, the PD specificity is poorly understood, and is hypothesized to act as a 1-phosphatase based on the activity of the human HsPPIP5Ks and the yeast homolog, Asp1 (Dollins et al. 2020; Gu et al. 2017; Pascual-Ortiz et al. 2018; Pöhlmann et al. 2014). Loss-of-function PP-InsP synthetic mutants in plants have been crucial in better understanding the role that PP-InsPs play in Pi sensing and homeostasis (Dong et al. 2019; Land et al. 2021; Zhu et al. 2019). VIP synthetic mutants, herein referred to as vih double mutants, have significantly reduced PP-InsP levels and have induced PSR gene expression, increased Pi accumulation, and greatly stunted growth (Dong et al. 2019; Zhu et al. 2019). Additionally, a knock-down of IPK1, ipk1, results in decreased PP-InsPs, elevated Pi accumulation, PSR gene induction and stunted growth (Kuo et al. 2018; Laha et al. 2015; Stevenson-Paulik et al. 2005). Similarly, ITPK1 loss-of-function mutant, itpk1, has a similarly reduced InsPs and PP-InsPs, and similar PSR phenotype ((Kuo et al. 2018; Laha et al. 2019)). These mutants further the understanding of the PSR, and the current of model PP-InsP-mediated PSR regulation.

PP-InsPs have also been implicated in plant hormone signaling, specifically in jasmonic acid (JA) and auxin pathways. InsP₆ has been identified as ligand of the transport inhibitor 1 (TIR1) auxin receptor complex (Tan et al. 2007). The auxin response of the itpk1 mutant was recently characterized, and itpk1 was found to have compromised auxin perception (Laha et al. 2020). InsP₇ and InsP₈ have also been found to bind to the coronatine insensitive-1 (COI1) jasmonate receptor complex (Laha et al. 2015, 2016). vih2, VIP1 knockdown mutants, have reduced InsP₈ levels, and have reduced JA perception and decreased defense responses to herbivorous insects (Laha et al. 2015).

In addition to their role in Pi sensing and hormone signaling, PP-InsPs have been implicated in a number of plant development and defense pathways, however, current studies have only focused on loss-of-function mutants. Applicant sought to generate gain-of-function ITPK1 and VIP2 transgenic plants, which Applicant reasoned would elevate InsPs and perturb native signaling pathways that utilize PP-InsPs. Such plants may, inter alia, further the understanding of the role of these signaling molecules. Here, Applicant describes overexpression of an ITPK1 construct and a KD only construct of VIP2 (VIP2KD), both of which result in distinct elevations in InsP and PP-InsP levels, with both having a shared elevation in InsP₈. Both types of transgenic plants have similar phenotypes and Pi-sensing responses, with key differences that suggest that the full complement of InsP₆, InsP₇ and InsP₈ are important in regulating the PSR. Together these transgenic plants provide a novel strategy for understanding the role of PP-InsPs in the context of Pi-sensing, plant development and defense responses.

### Results

### ITPK1 and VIP2KD Overexpression Delays Plant Growth and Development

To determine how elevated InsPs and PP-InsPs impact plant growth and development, Applicant generated transgenic plants overexpressing Inositol-Tetrakisphosphate 1-Kinase (ITPK1; EC 2.7.1.134, AT5G16760.1) and diphosphoinositol pentakisphosphate kinase VIP2 Kinase Domain (VIP2KD; EC: 2.7.4.21, AT5G15070.2) fused to a C-terminal GFP tag under control of the CaMV 35S promoter in Arabidopsis Col-0 plants. With the dual domain structure of the VIPs, the kinase domain alone drives InsP₈ synthesis. As overexpression of the full-length VIP2 construct (VIP2FL) gave rise to a small elevation in InsP₈, Applicant decided to overexpress the KD without the PD to drive synthesis of a large amount of InsP₈. For the ITPK1 OX overexpressor lines (ITPK1 OX), 15 transformants were obtained from the primary screen, and 6 lines with similar phenotypes were assayed for ITPK1-GFP fusion protein expression by Western blotting with an anti-GFP antibody, and fusion proteins of the expected molecular mass were detected (FIG. 1A). For the VIP2KD OX overexpressor lines (VIP2KD OX), 16 transformants were obtained from the primary screen, and 4 lines with similar phenotypes were assayed for VIP2KD-GFP fusion protein expression by Western blotting with an anti-GFP antibody, and fusion proteins of the expected molecular mass were detected (FIG. 1A). From the 6 lines of ITPK1 OX and 4 lines of VIP2KD OX, 2 lines of each type of transgenic were characterized in this study. Both ITPK1 OX lines have similar levels of protein accumulation (FIG. 1A) which correlates with their similar phenotypes. VIP2KD OX-1 has greater protein accumulation than VIP2KD OX-2 (FIG. 1A), which correlates with a more dramatic phenotype observed in VIP2KD OX-1 (FIG. 1B).

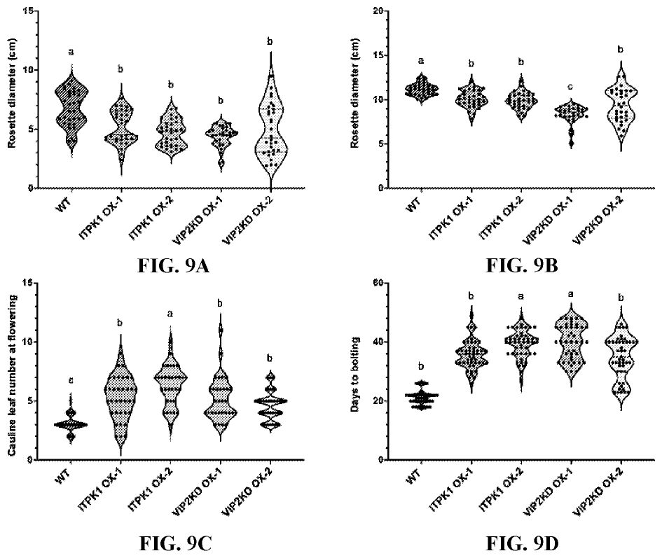

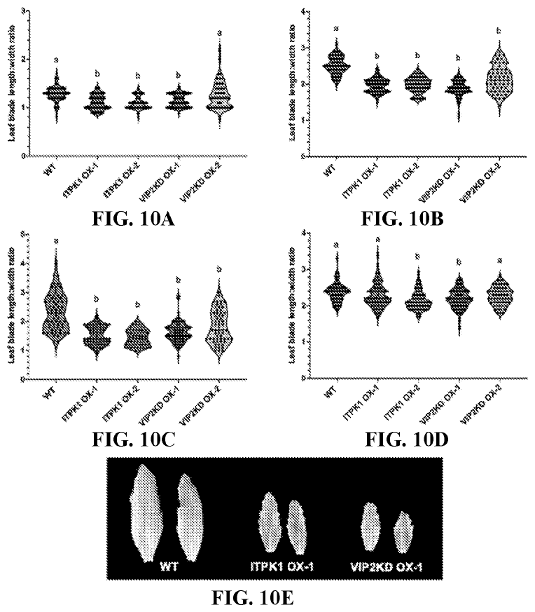

Applicant characterized both lines of ITPK1 OX and VIP2KD OX in comparison to WT (Col-0 ecotype). ITPK1 OX and VIP2KD OX plants exhibit various shared phenotypes throughout their development, including changes in leaf growth, coloration and morphology, onset of senescence, and delayed time to flowering, as compared to WT (FIGS. 1A-1G, 9A-9D, 10A-10E, 11, and 12A-12F). At 18 days of growth ITPK1 OX and VIP2KD OX plants have a reduced rosette diameter, and altered leaf morphology (FIG. 1C). At 24-days, ITPK1 OX and VIP2KD OX have a reduced rosette diameter (FIG. 9A), and altered leaf morphology (FIG. 1B-1D). By 35 days ITPK1 OX and VIP2KD OX plants contain several yellow leaves, indicative of early onset of senescence. The yellowing observed in these transgenic plants is distinct from the lesion formation seen in loss-of-function mutants containing reduced levels of PP-InsPs, as the vasculature of leaves remain green while the surrounding leaf tissue turns yellow over time. Also, at 35 days ITPK1 OX and VIP2KD OX plants contain darker green rosette leaves, as compared to WT (FIG. 1B). Also noted at 35 days, while WT plants have begun to flower, ITPK1 and VIP2KD are not, leading to a significantly delayed time to flowering (FIG. 1E, FIG. 9C-9D). ITPK1 OX and VIP2KD OX have significantly reduced rosette diameters at 35-days (FIG. 9B). At 50 days, most of the transgenic lines have reduced rosette diameters compared to WT (FIG. 1D). Yellow and dark green leaf coloration present in both types of transgenic lines and purple leaf coloration in VIP2KD OX is very pronounced. These purple leaves are suggestive of increased anthocyanin production, which usually occurs in response to perceived stresses, such as a low Pi environment. The leaf coloration phenotypes observed in the rosette leaves is also present in the cauline leaves (FIG. 10A). In addition to these phenotypes, throughout development ITPK1 OX and VIP2KD OX plants have a reduced leaf blade length to leaf blade width ratio (FIG. 10A-10D). In conclusion, ITPK1 OX and VIP2KD OX have similar phenotypes, but have key differences in leaf coloration, particularly after 35-days (FIG. 1A-1G).

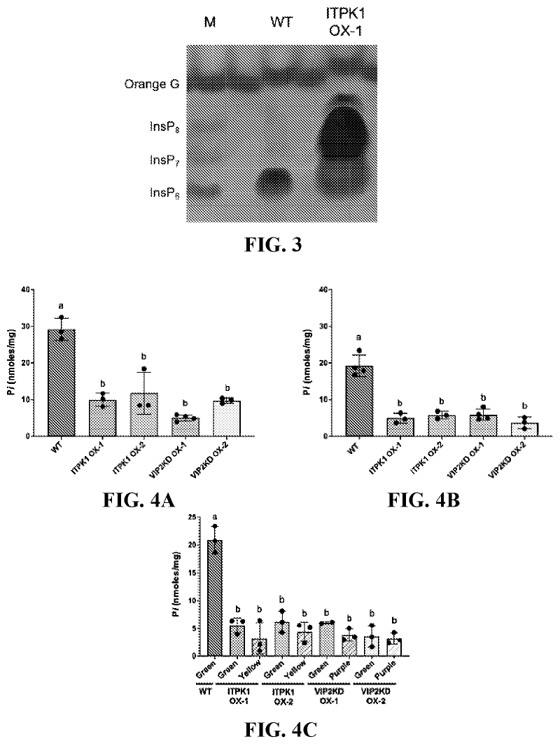

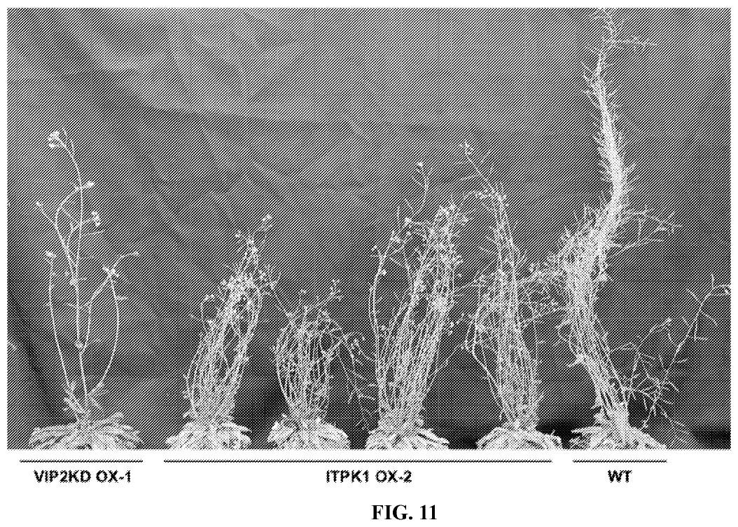

All transgenic lines have an increased time to flowering as measured by days to flowering and total number of cauline leaves at time of flowering (FIG. 1E), and increased time to bolting as measured as days to bolting. FITPK1 OX-1 and ITPK1 OX-2 have a significantly increased time to flowering of 42.6±2.2 days and 47.0±4.2 days respectively, compared to WT time to flowering of 28.2±2.2 days (FIG. 1D). VIP2KD OX-1 and VIP2KD OX-2 have an increase time to flowering of 48.0±6.6 and 41.5±7.1 days, respectively. Additionally, ITPK1 OX-1 and ITPK1 OX-2 exhibit altered inflorescence architecture (FIG. 1F-1G). ITPK1 OX lines develop stunted primary inflorescences and increased lateral inflorescence branching on the primary inflorescence, as well as increased secondary inflorescences, giving ITPK1 OX lines a “bushy” inflorescence phenotype (FIG. 1E-1F, FIG. 3). FIG. 10E shows an image demonstrating Cauline leaf phenotype of WT, ITPK1 OX-1 and VIP2KD OX-1 at 50-days. FIG. 11 shows the flowering phenotype of ITPK1 OX. Flowering phenotype of ITPK1 OX. Flowering phenotypes of ITPK1 OX-2 compared to age-match WT (right) and VIP2KD OX-2 (left) at 70-days.

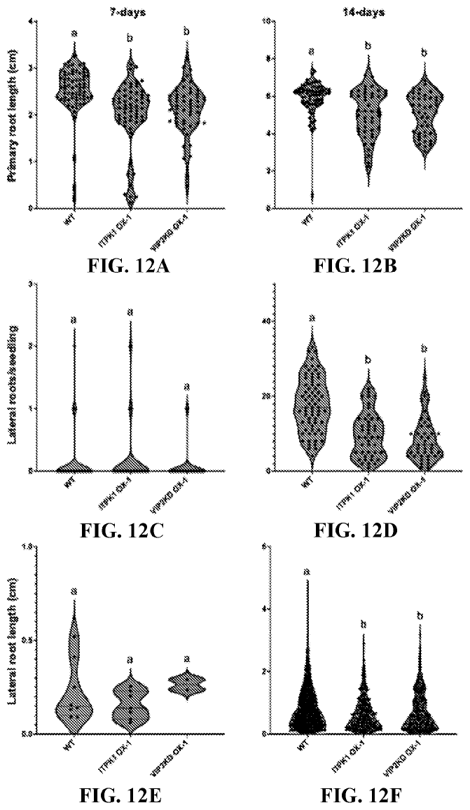

To determine if root development is altered in these two types of transgenic plants, Applicant quantified primary root and lateral root length, and number of lateral roots of ITPK1 OX-1 and VIP2KD OX-1 on ½×MS+1% sucrose plates at 7- and 14-days. Both ITPK1 OX-1 and VIP2KD OX-1 grown on Pi-sufficient plates have a reduced primary root length compared to WT at 7-days (FIG. 12A). At 14-days, both ITPK1 OX-1 and VIP2KD OX-1 have significantly reduced primary and lateral root length and reduced lateral root number per seedling compared to WT (FIG. 12B, 12D, 12F). These data show that ITPK1 OX-1 and VIP2KD OX-1 seedlings have an altered root development phenotype (FIG. 12A-12F).

Taken together, developmental transitions such as growth and onset of flowering have been impacted, at least suggesting that levels of PP-InsPs are key modulators of growth and development in these plants.

ITPK1 and VIP2KD OX Plants have Elevated PP-InsPs

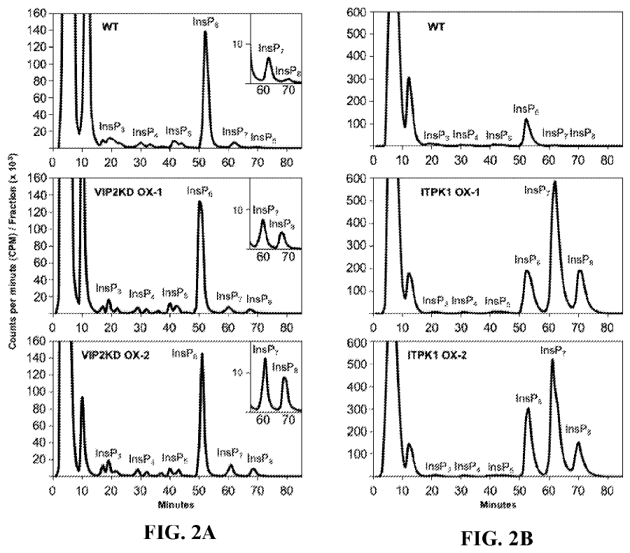

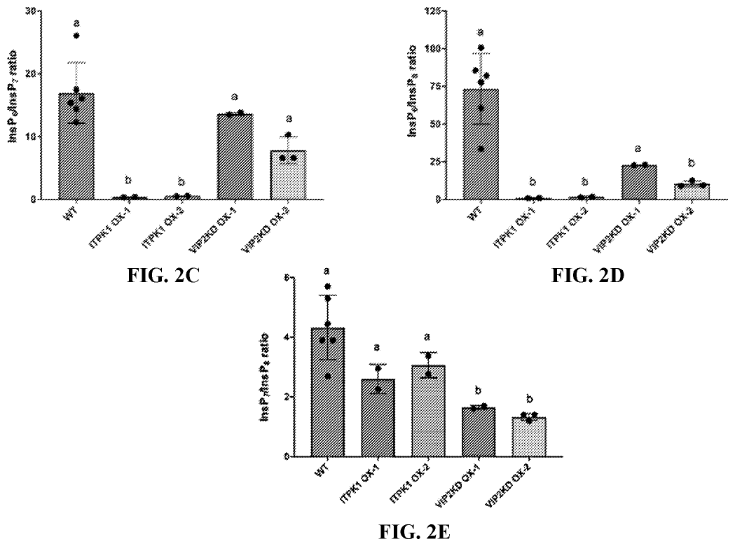

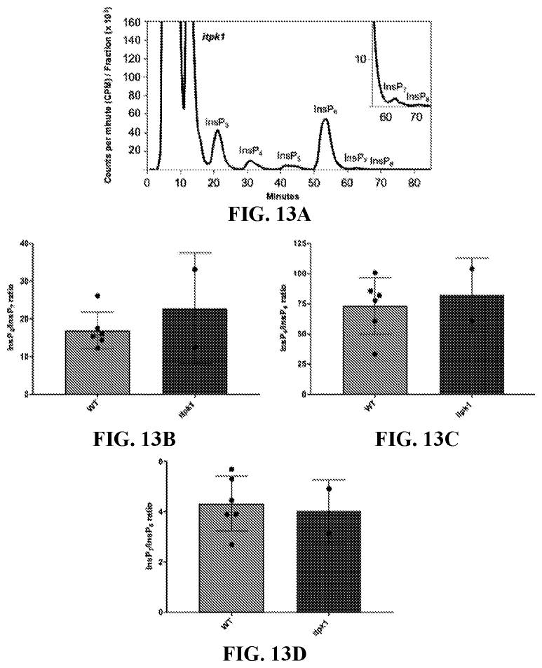

To determine the impact of ITPK1 and VIP2KD overexpression on InsP and PP-InsPs levels in planta, Applicant quantified PP-InsPs by radiolabeling with 3H-myo-inositol using previously described methods (Desai et al. 2014). Applicant also compared WT to previously described itpk1 loss-of-function mutants (FIG. 13A-13D). WT, VIP2KD OX and itpk1 traces were normalized based on fractions 16-85. Due to the large increase in total counts per minute (CPMs) in ITPK1 OX compared to other genotypes, Applicant did not normalize CPMs in ITPK OX and instead present this data as percentages and ratios. As expected, Applicant found that itpk1 mutants contained elevated levels of InsP₃ and decreased levels of InsP₆, InsP₇, and InsP₈ (FIG. 13A). In the ITPK1 OX lines, Applicant observed a large increase in InsP₅, InsP₆, InsP₇ and InsP₈ (FIG. 2B). ITPK1 OX-1 had 276%±65%, 17004%±10177%, and 29944%±15042% of the total WT InsP₆, InsP₇, and InsP₈ pool, respectively (Table 7). ITPK1 OX-2 had similar levels of InsPs as compared to ITPK1 OX-1, with 458%±25%, 14549%±1425%, and 21580%±3562% of the total WT pool of InsP₆, InsP₇ and InsP₈ pools, respectively.

In VIP2KD OX lines, Applicant observed a large increase only in InsP₈ levels. VIP2KD OX-1 had ˜400% of the total WT InsPs pool and comparable levels of InsP₆ and InsP₇ to WT (FIG. 2A, Table 7). Interestingly, VIP2KD OX-2, a line which exhibits less developmental delays compared to the VIP2KD OX-1 line, has a similar increase in InsP₈ (337%±99% of the total WT pool) yet had a significant decrease in InsP₆ compared to WT (42%±6% of the total WT InsP₆ pool).

TABLE 7
Average percent of InsPs as compared to the total WT pool.

| | ITPK1 OX-1 | | ITPK1 OX-2 | | VIP2KD OX-1 | | VIP2KD OX-2 | |
|---|---|---|---|---|---|---|---|---|
| | Average | Std Dev | Average | Std Dev | Average | Std Dev | Average | Std Dev |
| InsP₆ | 276% | ±65% | 458% | ±25% | 97% | ±3% | 42% | ±6% |
| InsP₇ | 17004% | ±10177% | 14549% | ±1425% | 110% | ±5% | 102% | ±17% |
| InsP₈ | 29944% | ±15042% | 21580% | ±3562% | 397% | ±57% | 337% | ±99% |

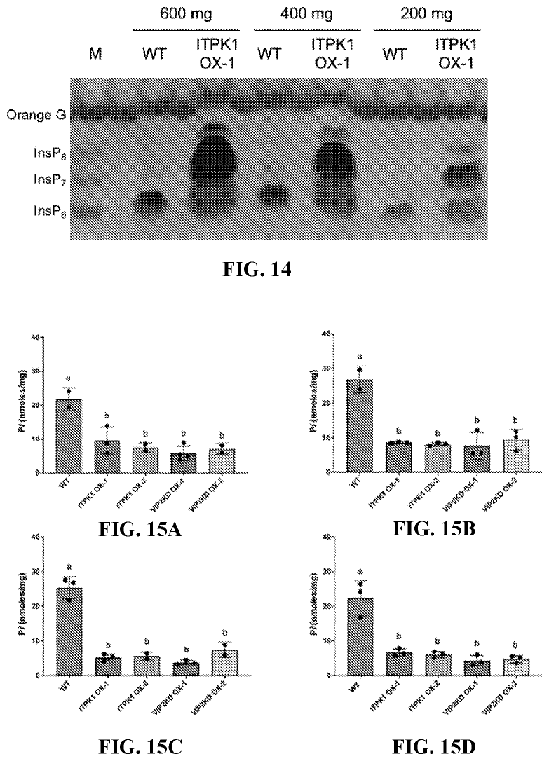

To determine whether these elevations in InsPs and PP-InsPs remained over development, Applicant analyzed InsP₆, InsP₇ and InsP₈ levels in 4-week rosette tissue from ITPK1 OX-1 soil-grown plants using 40% Polyacrylamide Gel Electrophoresis (PAGE) (FIG. 3, FIG. 14). Based on PAGE analysis, ITPK1 OX-1 has a large elevation in InsP₆, InsP₇ and InsP₈ compared to WT. In summary, elevations in InsPs and PP-InsPs persists throughout development in ITPK1 OX.

Examination of PP-InsP ratios is often used in studies of InsP mutants to compare the impact of loss- or gain-of functions. InsP₆/InsP₇, InsP₆/InsP₈ and InsP₇/InsP₈ ratios are presented in FIG. 2C-2E. ITPK1 OX lines have a significantly reduced InsP₆/InsP₇ and InsP₆/InsP₈ ratio, and a slightly reduced InsP₇/InsP₈ ratio (FIG. 2C-2E), which is not surprising given the large increase noted in InsP₆ levels. VIP2KD OX have a WT-like InsP₆/InsP₇ ratio which is expected as these molecules did not change, and a significantly reduced InsP₇/InsP₈ ratio. In general, the VIP2KD OX have a reduced InsP₆/InsPs ratio, but the difference is only significant in the OX-2 line (FIG. 2C-2E). Here, Applicant demonstrates the first example of elevated PP-InsPs in an overexpression system. Together, these data indicate that overexpression of ITPK1 and VIP2KD increase levels of InsP₈, although ITPK1 OX has an accompanying increase in InsP₆ and InsP₇. Applicant concludes both types of transgenic lines are useful to dissect the impact of elevated InsPs in plant signaling.

### Misregulation of PP-InsPs Impacts Pi Homeostasis

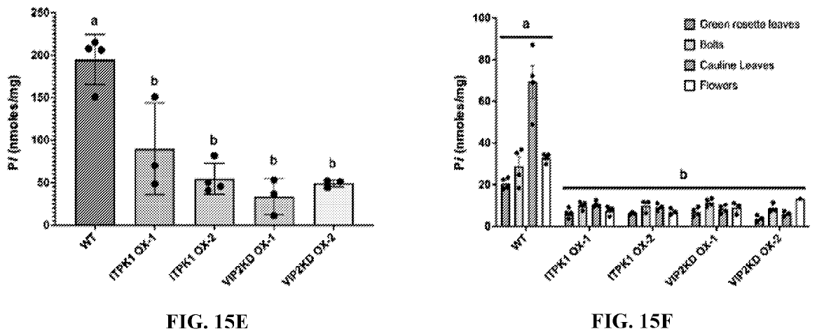

When PP-InsPs are greatly reduced, as in the case of vih mutants (Dong et al. 2019; Zhu et al. 2019) or when a synthetic gene is used to decrease PP-InsPs (Freed 2022), plants turn on the PSR and accumulate increase Pi as a result. Based on the current model of Pi sensing, Applicant hypothesized that both ITPK1 and VIP2KD OX plants would have reduced Pi accumulation, due to their elevation in PP-InsPs, specifically InsP₈. Applicant measured inorganic Pi in mixed rosette leaf tissue from soil-grown plants throughout development and found that both ITPK1 OX and VIP2KD OX have significantly decreased Pi accumulation across all queried time points, from 14-days to 70-days (FIG. 4A-4C, FIG. 15A-15D). In both ITPK1 OX and VIP2KD OX lines, inorganic Pi accumulation is two to four-fold lower than WT. Between ITPK1 OX and VIP2KD OX plants, there was no significant difference in Pi accumulation. Applicant also quantified Pi accumulation in senescing leaf tissue and reproductive tissues, where Applicant observed the same significant decrease in inorganic Pi (FIG. 15E-15F). Applicant concludes that ITPK1 OX and VIP2KD OX have a similar, decreased Pi accumulation over development.

To determine whether the yellow and purple rosette leaves of ITPK1 OX and VIP2KD OX had a different physiological status, Applicant quantified Pi levels in different colored rosette leaves from 50-day soil-grown plants (FIG. 4C). Similar to when querying mixed rosette tissue, Applicant found that yellow rosette tissue from ITPK1 OX plants and purple rosette tissue from VIP2KD OX plants have significantly reduced inorganic Pi levels as compared to WT.

Together, these results show that both ITPK1 OX and VIP2KD OX plants have significantly reduced inorganic Pi accumulation across development and tissue types.

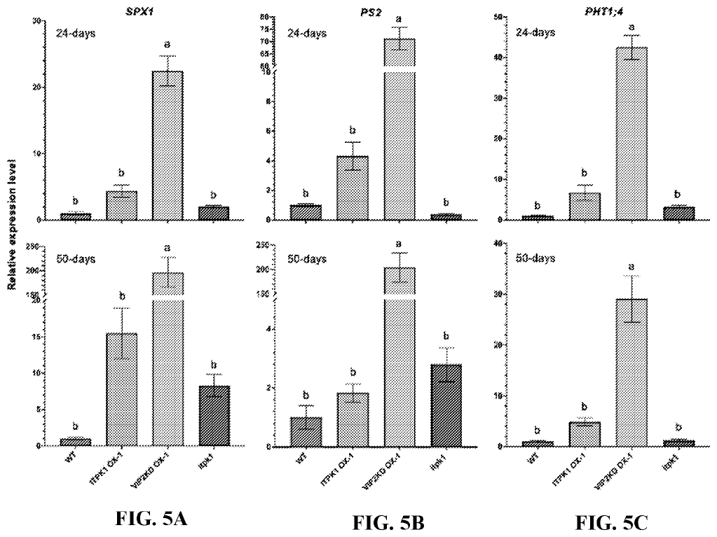

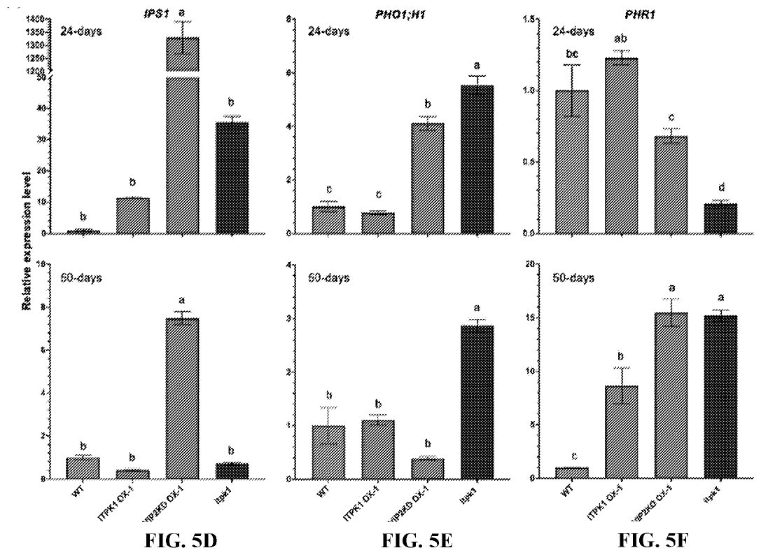

The model presented in the introduction of this Example (see also FIG. 8) shows how InsP₈ is hypothesized to control PSR gene expression. Based on the elevated PP-InsP levels and reduced inorganic Pi accumulation of both types ITPK1 OX and VIP2KD OX plants, Applicant hypothesized that PSR gene expression would be repressed in both types of transgenic plants. To test this, Applicant queried PSR marker genes in mixed rosette tissue in 24- and 50-day soil-grown ITPK1 OX-1 and VIP2KD OX-1 plants using qRT-PCR. At the 24-day time point both types of transgenics have similar phenotypes, and at 50-days key aspects of their phenotypes have diverged. Applicant also queried PSR marker genes in the itpk1 mutant as a control and to compare to previously published data (Kuo et al. 2018). Expression of these genes from itpk1 mutants recapitulates previously published data (Kuo et al. 2018), indicating these conditions are suitable for PSR gene expression analysis. Surprisingly, Applicant found that a majority of PSR marker genes were induced in both ITPK1 OX-1 and VIP2KD OX-2. For most of the PSR genes queried (SPX1, PS2, PHT1;4, IPS1; PHO1;H1 FIG. 5A-5E), ITPK1 OX-1 had an insignificant but consistent elevation in gene expression compared to WT in 24-day and 50-day old plants. VIP2KD OX-1 had a greater, significant increase in gene expression compared to the other mutants at both 24- and 50-days. However, VIP2KD OX induction of PHO1;H1 disappears at the 50-day time point. This trend was not observed in PHR1 (FIG. 5F). At 24-days, neither ITPK1 OX-1 or VIP2KD OX-1 have significantly different expression levels to WT, however, at the later time point, both types of transgenics have a significant induction of PHR1. Together, these data show that despite having elevated levels of PP-InsPs, there is evidence of PSR gene induction in both ITPK1 OX-1 and VIP2KD OX-1 plants. Furthermore, these data show that there are key differences in the level of induction in these genes between ITPK1 OX-1 and VIP2KD OX-1 plants.

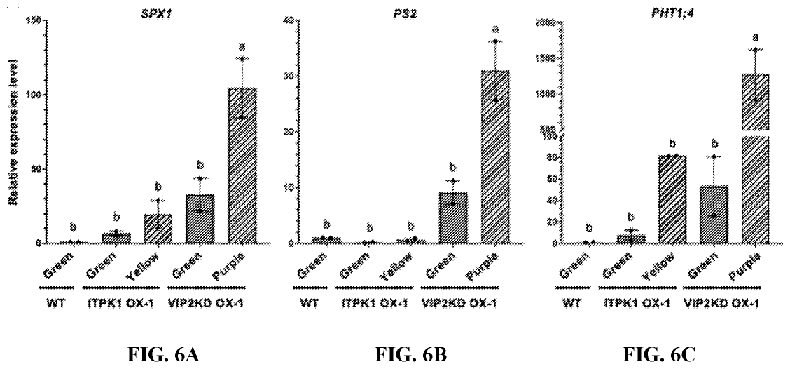

Because of the potentially different physiological status of the yellow and purple rosette leaves in ITPK1 OX and VIP2KD OX plants, Applicant queried a select number of PSR markers in yellow and purple rosette leaves in 50-day soil-grown plants (FIG. 6A-6C). VIP2KD OX-1 purple rosette leaves were the only sample to differ significantly from WT and other transgenic rosette leaves, with significant induction of all three queried genes.

Together, this data shows at least that despite having elevated levels of PP-InsPs, ITPK1 OX-1 and VIP2KD OX-1 plants have induced a subset of PSR genes. Additionally, the greater induction of PSR genes in VIP2KD OX-1 as compared to ITPK1 OX-1 suggests that the InsP₇/InsP₈ ratio may play a role in increasing PSR gene transcription in these transgenic lines.

### Jasmonic Acid Signaling is Impacted in ITPK1 OX and VIP2KD OX Lines

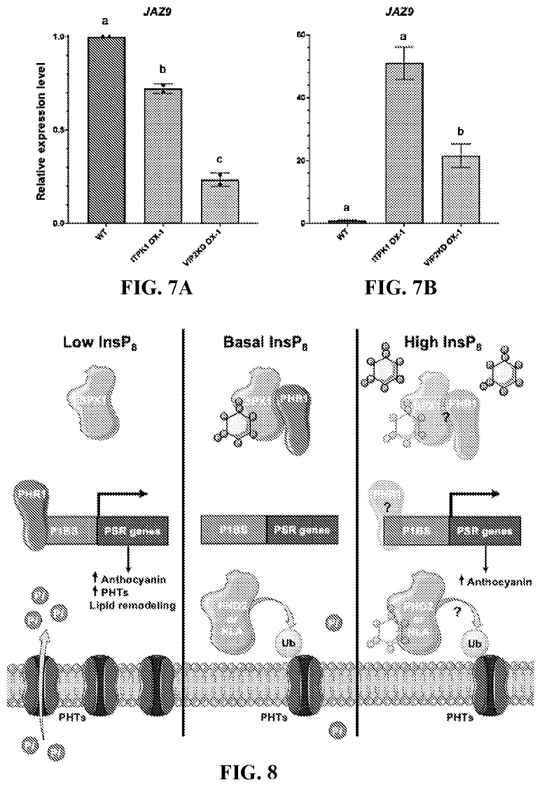

Previous work has identified Ins(1,2,4,5,6)P5 and InsP₈ as the likely ligand(s) in the jasmonic acid (JA) receptor complex (Mosblech et al. 2011; Sheard et al. 2010). Furthermore, plants with reduced InsP₈ have decreased JA-induced gene expression and are more susceptible to damage from herbivorous insects (Laha et al. 2015). To investigate whether elevating PP-InsPs JA regulation of gene expression, Applicant queried JA marker gene JAZ9 gene expression in green rosette tissue from 50-day soil-grown plants. Under regular growth conditions, both ITPK1 OX-1 and VIP2KD OX-1 leaves had significantly reduced JAZ9 expression (FIG. 7A). ITPK1 OX-1 was least impacted, with a ˜28% reduction in relative expression compared to WT, whereas VIP2KD OX-1 had a ˜77% reduction in relative expression compared to WT. Applicant conclude that under these conditions, JA signaling in ITPK1 OX and VIP2KD OX is downregulated.

Applicant and others in the lab observed phenotypic differences in surrounding WT plants when ITPK1 OX and VIP2KD OX were grown in close proximity, such as earlier senescence and increased anthocyanin production, suggesting ITPK1 OX and/or VIP2KD OX were volatizing compounds impacting plant growth. To test whether volatiles from ITPK1 OX and VIP2KD OX impacted WT growth, Applicant grew WT, ITPK1 OX-1 and VIP2KD OX-1 under a dome from 4-8-weeks. WT, ITPK1 OX-1 and VIP2KD OX-1 under domed conditions were characterized for their development, Pi-sensing status and JA signaling phenotypes. Applicant queried JAZ9 in 56-day green rosette tissue from WT, ITPK1 OX-1 and VIP2KD OX-1 grown under a dome for 4 weeks. Under domed conditions, ITPK1 OX-1 and VIP2KD OX-1 both had significantly induced JAZ9 expression (FIG. 7B). ITPK1 OX-1 had the most significant induction of JAZ9, with a 51-fold increase compared to WT. VIP2KD OX-1 had a significant 21-fold increase in JAZ9 expression compared to WT. Applicant conclude that concentrating potential volatiles from ITPK1 OX-1 and VIP2KD OX-1 plants can influence the regulation of a JA marker gene within these plants, while not impacting Pi-sensing signaling.

### Discussion

PP-InsPs are important messenger signaling molecules involved in nutrient sensing, energy homeostasis and hormone signaling pathways. Since most previous work has only focused on InsP and PP-InsP loss-of-function mutants, Applicant sought to understand how elevating PP-InsPs impacted plant growth and development. My work provides key insights into the role of PP-InsPs in Pi sensing, flowering time regulation, and JA signaling.

### Overexpression of ITPK1 and VIP2KD in Arabidopsis Leads to Large Elevations in PP-InsPs

Applicant hypothesized that overexpression of the final two enzymes in the PP-InsP synthesis pathway would drive elevated synthesis of PP-InsPs in Applicant's transgenic plants. A careful review of the literature suggests this is first example of such significant elevation in PP-InsPs in plants. Applicant shows that ITPK1 OX lines have elevated InsP₅, InsP₆, InsP₇, and InsP₈, and VIP2KD OX lines have elevated InsP₈. Furthermore, PAGE analysis of ITPK1 OX-1 indicates that elevated levels of PP-InsPs persist throughout development. Given ITPK1 impacts the InsP pathway in multiple places, it is not surprising that ITPK1 OX have large increases in multiple InsP and PP-InsP species. Because of these large InsP and PP-InsP increases, ITPK1 OX lines have 26 to 44-fold more CPM than other genotypes, and to prevent skewing WT and VIP2KD OX data, ITPK1 OX was not normalized. To address this, Applicant also analyzed the PP-InsP ratios, which do not necessitate normalization and are a commonly used approach to compare InsP mutants (Dong et al. 2019; Zhu et al. 2019). Analysis of PP-InsP ratios shows that the specific modulation of PP-InsPs in these transgenic plants leads to significantly reduced InsP₆/InsP₇ and InsP₆/InsP₈ in ITPK1 plants, and significantly reduced InsP₇/InsP₈ and InsP₆/InsP₈ ratio in VIP2KD OX plants. Taken together, the labelling and gel analysis shows that ITPK1 OX and VIP2KD OX plants have distinct elevations in PP-InsPs, with a common elevation in InsP₈.

Previous work by others reported only a slight increase in InsP₆ and InsP₇ in ITPK1 OX plants (Kuo et al. 2018), which may indicate that my ITPK1 transgenic protein was stabilized by its C-terminal fusion to GFP. Previous work that examined overexpression of VIP2 full-length protein in transgenic Arabidopsis revealed very small changes in InsP₈, which correlated with very little transgenic protein expression in these plants (Adepoju et al. 2019). Two other groups have overexpressed both VIP1 and VIP2 full length constructs which contained a mutation rendering the phosphatase domain inactive (Dong et al. 2019; Zhu et al. 2019). In principle these plants are hypothesized to be similar to the VIP2KD plants in that there is no phosphatase domain activity to limit InsPs synthesis and accumulation. In both of these studies there was no measurement or quantification of PP-InsPs, and developmental analyses were not reported (Dong et al. 2019; Zhu et al. 2019).

### ITPK1 and VIP2KD Overexpression Impact Pi-Sensing and Homeostasis

ITPK1 OX and VIP2KD OX have significantly reduced inorganic Pi levels in rosette leaves and reproductive tissue at all developmental time points queried (FIGS. 4A-4C and FIGS. 15A-15F). Other groups have reported similar decreases in Pi content and Pi uptake in roots of plate-grown OxITPK1 (Kuo et al. 2018). While this decrease in Pi accumulation in ITPK1 OX and VIP2KD OX are suggestive of repressed PSR gene expression, Applicant surprisingly found that there was a consistent induction of PSR genes in both ITPK1 OX and VIP2KD OX at 24 and 50 days (FIG. 4A-4B). When comparing PSR gene expression of the two types of transgenic plants, VIP2KD OX had a consistently greater induction than ITPK1 OX. Additionally, for a majority of the PSR genes queried, VIP2KD OX had a greater induction than the well-characterized itpk1 mutant, which is considered to have a constitutive and upregulated PSR physiological status. Applicant also shows that purple rosette tissue from VIP2KD OX have significantly induced PSR gene expression compared to green rosette tissue, which suggests that these purple leaves have an advanced PSR physiological status.

This induction of PSR gene expression, despite the plant having elevated levels of InsP₈, suggests a more complex model for InsP₈-mediated Pi sensing than is currently described (Azevedo & Saiardi 2017; Wild et al. 2016). In the current model, InsP₈ is thought to be the primary ligand for facilitating PHR1: SPX1 interactions, and negatively regulating PSR gene induction. In the absence of InsP₈, PSR gene expression is induced via PHR1. However, both ITPK1 OX and VIP2KD OX plants have elevated InsP₈ and elevated PSR gene expression. Previous work characterizing loss-of-function vip1-2/vip2-2 mutants (Land et al. 2021) has shown that small changes in InsP₇ and InsP₈ levels between vip and vih mutants result in near opposite PSR gene expression and Pi accumulation phenotypes. Applicant proposes that alterations in PP-InsPs impact genes from multiple pathways (the PSR (FIGS. 5-6) and JA signaling (FIG. 7A-B)) which have an impact on the plant's phenotype. In ITPK1 OX and VIP2KD OX, induction of PSR genes does not result in elevated Pi accumulation, suggesting that some of these gene products are being targeted for degradation. PHO2, an E2 ubiquitin-conjugating enzyme, is a well-characterized enzyme involved in PSR regulation. In response to Pi availability, PHO2 mediates Pi transporter degradation (Huang et al. 2013; Liu et al. 2012). PHO2 is thought to act as a secondary form of regulation, degrading Pi transporters under Pi-sufficient conditions, and being blocked from degrading Pi transporters under low Pi conditions (Bari et al. 2006; Huang et al. 2013; Liu et al. 2012). With the disconnect between PSR gene expression and Pi accumulation in ITPK1 OX and VIP2KD OX, it is conceivable the PHO2-mediated Pi transporter degradation may be elevated in these plants. It is noteworthy to add that another E3 Ubiquitin ligase encoded by NLA1 or NLA2 includes an SPX domain, and NLA is also known to regulate phosphate transporter stability under nitrogen limitation (Hsieh et al. 2009; Kant et al. 2011; Peng et al. 2007; Yaeno & Iba 2008). Given that InsP₈ likely has the capability to bind to NLA, this Ubiquitin ligase might also be affected in ITPK1 and VIP2KD plants. Future studies should investigate protein stability and accumulation in these two transgenic types. Furthermore, the Arabidopsis genome encodes 20 SPX domain-containing proteins, many of which have functions related to Pi transport, and nutrient- and energy-sensing (Table 8). As elevated InsP₈, and possibly other InsPs, could bind and modulate the activity of these SPX domain-containing proteins, one of more these may be affecting altered PSR gene regulation and Pi accumulation within the ITPK1 OX and VIP2KD OX plants.

The results of this study reveal an unexpected, and complex regulation of the PSR by the PP-InsPs, and provide a novel system for future studies on PSR regulation in plants. With the limited Pi supply and growing global population, ITPK1 OX and VIP2KD OX plants offer key insights and potential strategy for developing plants that uptake less Pi without a large growth trade-off.

TABLE 8
Arabidopsis thaliana proteins with SPX domains. Adapted and updated from
(Secco et al. 2012; Wang et al. 2021).

| SPX | | | InsP | | |
|---|---|---|---|---|---|
| domain | Protein | Locus | binding | | |
| type | Name | identifier | dependency | Function | Reference |
| SPX | SPX1 | At5g20150 | InsP₆, InsP₇, | Negative regulator | (Dong et al. 2019; |
| | | | InsP₈ | of PSR gene | Duan et al. 2008; |
| | | | | expression; positive | Lv et al. 2014; |
| | | | | regulator of leaf | Puga et al. 2014; |
| | | | | senescence, and | Wang et al. 2014; |
| | | | | salicylic acid | Wild et al. 2016; |
| | | | | signaling | Yan et al. 2019; |
| | | | | | Zhou et al. 2015) |
| | SPX2 | At2g26660 | Unknown | Negative regulator | (Duan et al. 2008; |
| | | | | of PSR gene | Ruan et al. 2019) |
| | | | | expression | |
| | SPX3 | At2g45130 | Unknown | Negative regulator | (Duan et al. 2008) |
| | | | | of PSR gene | |
| | | | | expression; | |
| | | | | negative regulator | |
| | | | | of SPX1 | |
| | SPX4 | At5g15330 | Unknown | Negative regulator | (Duan et al. 2008; |
| | | | | of PHR1-dependent | Osorio et al. 2019) |
| | | | | and PHR1- | |
| | | | | independent PSR; | |
| | | | | not involved in | |
| | | | | short-term recovery | |
| | | | | after Pi resupply | |
| SPX- | PHO1 | At3g23430 | Unknown | Pi transporter; | (Hamburger et al. |
| EXS | | | | facilitates transport | 2002; Poirier et al. |
| | | | | of Pi from root to | 1991; Rouached et |
| | | | | shoot | al. 2011; Stefanovic |
| | | | | | et al. 2007; Wang |
| | | | | | et al. 2004) |
| | PHO1; H1 | At1g68740 | Unknown | Pi transporter; | (Stefanovic et al. |
| | | | | facilitates transport | 2007; Wang et al. |
| | | | | of Pi from root to | 2004) |
| | | | | shoot | |
| | PHO1; H2 | At2g03260 | Unknown | Pi transporter | (Wang et al. 2004) |
| | PHO1; H3 | At1g14040 | Unknown | Pi transporter | (Wang et al. 2004) |
| | PHO1; H4/ | At4g25350 | Unknown | Putative Pi | (Kang & Ni 2006; |
| | SHB1 | | | transporter; | Wang et al. 2004; |
| | | | | Involved in | Zhou & Ni 2009; |
| | | | | cryptochrome | Zhou et al. 2009) |
| | | | | signaling, regulates | |
| | | | | seed development | |
| | PHO1; H5 | At2g03240 | Unknown | Pi transporter | (Wang et al. 2004) |
| | PHO1; H6 | At2g03250 | Unknown | Pi transporter | (Wang et al. 2004) |
| | PHO1; H7 | At1g26730 | Unknown | Pi transporter | (Wang et al. 2004) |
| | PHO1; H8 | At1g35350 | Unknown | Pi transporter | (Wang et al. 2004) |
| | PHO1; H9 | At3g29060 | Unknown | Pi transporter | (Wang et al. 2004) |
| | PHO1; H10 | At1g69480 | Unknown | Pi transporter; | (Ribot et al. |
| | | | | involved in biotic | 2008b, a; Wang et |
| | | | | and abiotic stress | al. 2004) |
| | | | | signaling pathways | |
| SPX- | NLA/ | Atlg02860 | Unknown | E3 ubiquitin ligase; | (Hsieh et al. 2009; |
| RING | BAHI | | | regulates Pi | Kant et al. 2011; |
| | | | | homeostasis under | Lin et al. 2013; |
| | | | | N starvation; | Park et al. 2018; |
| | | | | involved in | Peng et al. 2007; |
| | | | | regulation of | Yaeno & Iba 2008) |
| | | | | anthocyanin | |
| | | | | synthesis | |
| | NLA2 | At2g38920 | Unknown | Predicted E3 | |
| | | | | ubiquitin ligase | |
| SPX- | PHT5; 1 | At1g63010 | Unknown | Pi transporter; | (Liu et al. 2016; |
| MFS | | | | involved in | Luan et al. 2019) |
| | | | | transport of Pi | |
| | | | | between cytoplasm | |
| | | | | and vacuole | |
| | PHT5; 2 | At4g11810 | Unknown | Pi transporter; | (Liu et al. 2016; |
| | | | | involved in | Luan et al. 2019) |
| | | | | transport of Pi | |
| | | | | between cytoplasm | |
| | | | | and vacuole | |
| | PHT5; 3 | At4g22990 | Unknown | Pi transporter; | (Liu et al. 2016; |
| | | | | involved in | Luan et al. 2019) |
| | | | | transport of Pi | |
| | | | | between cytoplasm | |
| | | | | and vacuole | |

### Elevations in PP-InsPs Impacts Development and Hormone Signaling Pathways

ITPK1 OX and VIP2KD OX plants exhibit distinct phenotypes, including changes in leaf coloration and morphology, onset of senescence, and delayed time to flowering, as compared to WT. From 35-days, ITPK1 OX and VIP2KD OX plants have several yellow rosette leaves indicative of an altered senescence pattern and distinct from the yellow lesion formation seen in loss-of-function InsP mutants. From 50-days, ITPK1 OX plants have pronounced dark green and yellow rosette leaves, and VIP2KD OX plants have dark purple rosette leave, suggestive of increased anthocyanin accumulation. Anthocyanin production is increased when plants are under perceived stress, such as low Pi (Chien et al.; Chiou & Lin 2011; Crombez et al. 2019). Others have identified VIP1/VIH2 as a gene involved in flowering time regulation in Arabidopsis (Zhang 2017). Zhang et al., 2017 characterized time to flowering vih2 and VIP1OX lines, and found that vih2 loss-of-function mutants have decreased time to flowering, and VIP1OX lines have delayed time to flowering. This group suggested this difference in flowering time was related to the InsP₇/InsP₈ ratio. Similarly, Applicant observed a significant delay in time to flowering in ITPK1 OX and VIP2KD OX, which all have reduced InsP₇/InsP₈ ratios. Additional evidence comes from analysis of the itpk1 mutant, which has decreased InsP₅, InsP₆, InsP₇ and InsP₈, but no change in InsP₇/InsP₈ ratios, and no significant difference in time to flowering compared to WT (FIG. 1F, FIG. 13B-13D).

Given the interaction between PP-InsPs and plant hormone pathways, we used qRT-PCR to quantify JA marker gene expression in ITPK1 OX and VIP2KD OX. When querying undomed green rosette tissue in both types of transgenics, Applicant observed a significant repression of the JA marker gene JAZ9 in ITPK1 OX-1 plants, and a further significant repression of this same gene in VIP2KD OX-1 plants. These results suggest that JA signaling is dampened in both types of transgenics, and more greatly impacted in VIP2KD OX-1. However, when both types of transgenics are domed, which can concentrate any released plant volatiles, JAZ9 is significantly induced, and more greatly induced in ITPK1 OX-1. Only one study has investigated the cross-talk between PP-InsP and JA signaling, and showed that vih2-4 mutants with decreased InsP₈ have reduced resistance to herbivorous insects and decreased JA marker gene expression (Laha et al. 2015). While the ITPK1 OX and VIP2KD OX transgenics and vih2-4 mutants have opposite InsPs levels but similar JA signaling gene expression, this suggests a more complex model of PP-InsP-JA crosstalk than is currently proposed. Future work will investigate JA levels, herbivorous insect defense and volatile production in ITPK1 OX and VIP2KD OX plants.

PP-InsPs have also been implicated in auxin perception and signaling (Laha et al. 2020; Tan et al. 2007). Others have demonstrated that InsP₅, InsP₆, and InsP₇ can bind to the ASK1-TIR1 auxin receptor complex (Laha et al. 2020; Tan et al. 2007). The altered inflorescence architecture observed ITPK1 OX plants (FIG. 1F-1G) is suggestive of altered auxin and sugar signaling (Barbier et al. 2015, 2017; Mason et al. 2014). Recent studies have investigated the relationship between InsP₇, InsP₈ and auxin signaling in itpk1 mutants, finding that itpk1 mutants with reduced, InsP₇, and InsP₈ have compromised auxin signaling (Laha et al. 2020). Given ITPK1 OX plants have elevated InsP₇ and InsP₈, based on previous studies Applicant would predict ITPK1 OX plants to have elevated auxin signaling. Together, this data at least shows that elevating InsP₈ levels in Arabidopsis delays developmental transitions and impacts plant hormone signaling pathways.

### Experimental Procedures

### Cloning and Transformation

ITPK1 and VIP2KD from Arabidopsis thaliana were PCR amplified for ligation in Gateway pENTR/D-TOPO destination vector. Using the Gateway® LR Clonase™ II kit (Invitrogen Corp., Carlsbad, CA), ITPK1 and VIP2KD were recombined with Gateway plant destination vectors pK7FWG2 (C-terminus ITPK1-GFP and VIP2KD-GFP) (VIB UGent Center for Plant Systems Biology). E. coli transformed with these constructs was selected on antibiotic media. Plasmids purified from resulting colonies were sequencing for verification of correct cloning. Agrobacterium (strain GV3101) was transformed with these constructs and were used to transform Arabidopsis thaliana (ecotype Col-0) plants.

### Plant Materials, Growth Conditions and Flowering Time Scoring

itpk1 (SAIL_65_D03) T-DNA mutants were obtained from the Arabidopsis Biological Resource Center (ABRC, Columbus, OH, USA). Arabidopsis seeds were stratified in distilled water at 4° C. for 3 days and sown in soil under long day conditions (16 hours light/8 hours dark, 55% humidity, day/night temperature 23/21° C. and 120 μE light intensity). For radiolabeling experiments seeds were sterilized using 100% ethanol for one minute, transferred to a 30% (v/v) Clorox solution for 5 minutes and washed five times with ddH₂O. Seeds were placed in 0.2% agar and stratified for 3 days at 4° C. Seedlings were transferred to multiwell plates containing semi-solid media (0.5×MS media+0.2% agar). Flowering time was scored for each plant by the number of days until the first flower opened and number of cauline leaves when the first flower opened. All flowering experiments were repeated in duplicate with n=12 to 32 plants per replicate.

### Protein Blots of GFP-Fusion Proteins

Protein blots were performed as previously reported (Burnette et al. 2003). Leaf tissue from soil grown plants from was pulverized in liquid nitrogen, homogenized, and separated from cell debris. SDS-bromophenol blue loading dye was added to the extracted proteins and boiled for 5 minutes at 85° C. After a subsequent centrifugation, the supernatant was loaded onto a polyacrylamide gel; equal amounts of protein were added from each sample. For western blotting, a 1:5000 dilution of anti-GFP antibody (Invitrogen Molecular Probes, Eugene, OR) and a 1:2000 dilution of secondary goat anti-rabbit horseradish peroxidase antibody (Bio-Rad Laboratories, Hercules, CA) were used to detect GFP.

### InsP and PP-InsP Profiles of Arabidopsis Seedlings

Arabidopsis seedlings were grown in 0.5×MS+0.2% w/v agarose, pH 5.7 with a 16 h light, 8 h dark cycle, 30 μE light intensity. After 2 weeks of growth, 50 μCi of [³H]-myo-Inositol (20 Ci/mmol, American Radiolabeled Chemicals, St. Louis, MO, USA) was added to 24 seedlings in the same media. After 5 days of incubation under constant light, the seedlings were rinsed 3 times with water, frozen, then thawed and extracted with 1 M perchloric acid+3 mM EDTA+0.1 mg/mL InsP₆ (Sigma Aldrich, St. Louis, MO, USA) by douncing with a plastic pestle on ice. The extract was then centrifuged for 5 min at maximum speed, the supernatant was transferred to a microcentrifuge tube and neutralized to pH 6-8 with 1 M potassium carbonate+3 mM EDTA. The tubes were loosely capped and incubated on ice for 2 h, then centrifuged at maximum speed for 5 min. The supernatant was transferred to a microcentrifuge tube and concentrated under a stream of nitrogen. Inositol polyphosphates were separated by HPLC using a 125×4.6 mm Partisphere SAX-column (MAC-MOD Analytical, Chadds Ford, PA, USA), and eluted with a previously described ammonium phosphate gradient (Azevedo and Saiardi, 2006) Eighty-five fractions were collected and radioactivity was measured by liquid scintillation counting (Beckman Coulter, New Brunswick, NJ, USA). WT, VIP2KD OX and itpk1 traces were normalized based on fractions 16-85, as previously described (Desai et al. 2014; Land et al. 2021). Due to the large increase in a majority of the InsP and PP-InsP species in ITPK1 OX, Applicant did not normalize CPMs and instead present this data as percentages and ratios.

### Extraction, Isolation of InsPs and PAGE Analysis

InsPs were extracted from 1 g of 4-week-old soil-grown rosette leaves as described in (Wilson et al. 2015). Samples were pulverized in liquid nitrogen, then extracted in a glass dounce homogenizer with 3 mL of 1 M ice-cold perchloric acid (PA). The volume was brought to 9 ml with cold PA and incubated on ice for 15-20 min. Samples were then centrifuged at 16 000 g for 10 min at 4° C., and the supernatant was transferred to a new tube containing 10 mg of PA-equilibrated TiO₂ beads (5 μM Titansphere; GLSciences, Japan) and rotated at 4° C. for 20 min. After centrifugation at 3200 g for 2 min at room temperature, the supernatant was discarded and TiO₂ beads were washed twice with cold perchloric acid. InsPs were eluted twice with 0.3 mL of 10% ammonium hydroxide for 5 min rotating at 4° C. Pooled eluates were concentrated under a stream of nitrogen gas to 20 μL at room temperature, then the samples were mixed with orange G loading buffer and resolved on a 35% polyacrylamide/Tris-borate-EDTA gel (Losito et al. 2009).

### Pi Accumulation Assays

Pi was extracted from ˜55-65 mg from Arabidopsis tissue of various ages. Samples were pulverized in liquid nitrogen. A 1:10 ratio of 1% acetic acid was added to each sample, which was vortexed and incubated on ice, and centrifuged. Assays were performed on Pi extracts using a modified micro-titer assay as previously reported (Ames, 1966). 50 μL of the supernatant and 1 mL of working reagent (5% w/v FeH₁₄O₁₁S·7H₂O, 1% w/v (NH₄)2MoO₄, 1N H₂SO₄ aq.) was incubated for an hour. Samples were placed in quartz cuvettes and absorbance at 660 nm was measured using a spectrophotometer. (Model, Manufacturer). All Pi concentrations were calculated based on a standard curve from a set of standards made with known Pi concentrations.

### RNA Isolation and Quantitative Real-Time PCR

RNA was extracted from 10-day-old whole seedlings grown on ½×MS media containing 1% sucrose and 0.8% agar (Plant RNeasy kit (Qiagen)). During this process, samples were DNAse treated with RNase-free DNase (Qiagen). cDNA was generated using the iScript cDNA Synthesis Kit (Bio-Rad Laboratories, USA) using manufacturer's instructions. Quantitative PCR was performed using materials and methods previously described (Donahue et al., 2010). All primer sequences used in the qRT-PCR analysis can be found in Table 9. Relative expression was calculated using the ΔΔCT method. Expression data was normalized to PEX4 and relative to the fold change in WT.

TABLE 9

| Primer | Sequence |
|---|---|
| PEX4 F | 5′ CTTAACTGCGACTCAGGGAATCTTCTAAG 3′ (SEQ ID NO: 13) |
| PEX4 R | 5′ TCATCCTTTCTTAGGCATAGCGGC 3′ (SEQ ID NO: 14) |
| SPX1 F1 | 5′ GATTCCATTGTTGGAGCAAGA 3′ (SEQ ID NO: 15) |
| SPX1 R1 | 5′ AATCTGTTAGCTTCTTCTATTGTA 3′ (SEQ ID NO: 16) |
| PHT1; 4 F | 5′ GAACGGTCCCAATAGTTTAGGTGAT 3′ (SEQ ID NO: 17) |
| PHT1; 4 R | 5′ GAGTTGCTAGAGACAAGGAGAAAGAAA 3′ (SEQ ID NO: 18) |
| PS2 F | 5′ TCTTGAGAACAATCCCAATTCATC 3′ (SEQ ID NO: 19) |
| PS2 R | 5′ CCTAAGCTCACACCCTAAATCATGT 3′ (SEQ ID NO: 20) |
| IPS1 F | 5′ TTTGGAGAATAGTCAGACCAGTGC 3′ (SEQ ID NO: 21) |
| IPS1 R | 5′ TCACTATAAAGAGAATCGGAAGCA 3′ (SEQ ID NO: 22) |
| PHO1; H1 F | 5′ TACCGATTGGAGAATGAGCATCTAA 3′ (SEQ ID NO: 23) |
| PHO1; H R | 5′ TTAGTCTTCTTCATCCACTTCTCTGAAAG 3′ (SEQ ID NO: 24) |
| JAZ9 F | 5′ GAGGTTAACGATGATGCTGTC 3′ (SEQ ID NO: 25) |
| JAZ9 R | 5′ CCTGGAAATCTGAAGAAGGC 3′ (SEQ ID NO: 26) |

### REFERENCES FOR EXAMPLE 1

Adepoju O, Williams S P, Craige B, Cridland C A, Sharpe A K, et al. 2019. Inositol Trisphosphate Kinase and Diphosphoinositol Pentakisphosphate Kinase Enzymes Constitute the Inositol Pyrophosphate Synthesis Pathway in Plants. bioRxiv. 724914
Azevedo C, Saiardi A. 2017. Eukaryotic Phosphate Homeostasis: The Inositol Pyrophosphate Perspective. Trends Biochem. Sci. 42(3):219-31
Barbier F F, Dun E A, Beveridge C A. 2017. Apical dominance. Curr. Biol. 27(17):R864-65
Barbier F F, Lunn J E, Beveridge C A. 2015. Ready, steady, go! A sugar hit starts the race to shoot branching. Curr. Opin. Plant Biol. 25:39-45
Bari R, Pant B D, Stitt M, Scheible W R. 2006. PHO2, MicroRNA399, and PHR1 Define a Phosphate-Signaling Pathway in Plants. Plant Physiol. 141(3):988-99
Burnette R N, Gunesekra B M, Gillaspy G E. 2003. An Arabidopsis inositol 5-phosphatase gain-of-function alters abscisic acid signaling. Plant Physiol. 132(2):1011-19
Bustos R, Castrillo G, Linhares F, Puga M I, Rubio V, et al. 2010. A central regulatory system largely controls transcriptional activation and repression responses to phosphate starvation in Arabidopsis. PLoS Genet. 6(9):e1001102
Chien P-S, Chiang C-P, Leong S J, Chiou T-J. Sensing and Signaling of Phosphate Starvation: From Local to Long Distance
Chien P S, Chiang C P, Leong S J, Chiou T J. 2018. Sensing and Signaling of Phosphate Starvation: From Local to Long Distance
Chiou T-J, Lin S-I. 2011. Signaling Network in Sensing Phosphate Availability in Plants. Annu. Rev. Plant Biol. 62(1):185-206
Cordell D, White S. 2011. Peak Phosphorus: Clarifying the Key Issues of a Vigorous Debate about Long-Term Phosphorus Security. Sustain. 2011, Vol. 3, Pages 2027-2049. 3(10):2027-49
Crombez H, Motte H, Beeckman T. 2019. Tackling Plant Phosphate Starvation by the Roots. Dev. Cell. 48(5):599-615
Desai M, Rangarajan P, Donahue J L, Williams S P, Land E S, et al. 2014. Two inositol hexakisphosphate kinases drive inositol pyrophosphate synthesis in plants. Plant J. 80(4):642-53
Dollins E D, Bai W, Fridy P C, Otto J C, Neubauer J L, et al. 2020. Vip1 is a kinase and pyrophosphatase switch that regulates inositol diphosphate signaling. Proc. Natl. Acad. Sci. U.S.A 117(17):9356-64
Dong J, Ma G, Sui L, Wei M, Satheesh V, et al. 2019. Inositol Pyrophosphate InsP8 Acts as an Intracellular Phosphate Signal in Arabidopsis. Mol. Plant. 12(11):1463-73
Duan K, Yi K, Dang L, Huang H, Wu W, Wu P. 2008. Characterization of a sub-family of Arabidopsis genes with the SPX domain reveals their diverse functions in plant tolerance to phosphorus starvation. Plant J. 54(6):965-75
Freed C P. 2022. Inositol Pyrophosphate Phosphatases as Key Enzymes to Understand and Manipulate Phosphate Sensing in Plants
Gokhale N A, Zaremba A, Shears S B. 20011. Receptor-dependent compartmentalization of PPIP5K1, a kinase with a cryptic polyphosphoinositide binding domain. Biochem. J. 434(3):415-26
Gu C, Nguyen H N, Hofer A, Jessen H J, Dai X, et al. 2017. The significance of the bifunctional kinase/phosphatase activities of diphosphoinositol pentakisphosphate kinases (PPIP5Ks) for coupling inositol pyrophosphate cell signaling to cellular phosphate homeostasis. J. Biol. Chem. 292(11):4544-55
Hamburger D, Rezzonico E, Petétot J M D C, Somerville C, Poirier Y. 2002. Identification and characterization of the Arabidopsis PHO1 gene involved in phosphate loading to the xylem. Plant Cell. 14(4):889-902
Herrera-Estrella L, López-Arredondo D. 2016. Phosphorus: The Underrated Element for Feeding the World. Trends Plant Sci. 21(6):461-63
Hsieh L C, Lin S I, Shih A C C, Chen J W, Lin W Y, et al. 2009. Uncovering small RNA-mediated responses to phosphate deficiency in Arabidopsis by deep sequencing. Plant Physiol. 151(4):2120-32
Huang T K, Han C L, Lin S I, Chen Y J, Tsai Y C, et al. 2013. Identification of downstream components of ubiquitin-conjugating enzyme PHOSPHATE2 by quantitative membrane proteomics in Arabidopsis roots. Plant Cell. 25(10):4044-60
Kang X, Ni M. 2006. Arabidopsis SHORT HYPOCOTYL UNDER BLUE1 contains SPX and EXS domains and acts in cryptochrome signaling. Plant Cell. 18(4):921-34
Kant S, Peng M, Rothstein S J. 2011. Genetic regulation by NLA and microRNA827 for maintaining nitrate-dependent phosphate homeostasis in Arabidopsis. PLoS Genet. 7(3):
Kuo H F, Hsu Y Y, Lin W C, Chen K Y, Munnik T, et al. 2018. Arabidopsis inositol phosphate kinases IPK1 and ITPK1 constitute a metabolic pathway in maintaining phosphate homeostasis. Plant J. 95(4):613-30
Laha D, Johnen P, Azevedo C, Dynowski M, Weiß M, et al. 2015. VIH2 regulates the synthesis of inositol pyrophosphate InsP8 and jasmonate-dependent defenses in Arabidopsis. Plant Cell. 27(4):1082-97
Laha D, Parvin N, Dynowski M, Johnen P, Mao H, et al. 2016. Inositol polyphosphate binding specificity of the jasmonate receptor complex
Laha D, Parvin N, Hofer A, Giehl R F H, Fernandez-Rebollo N, et al. 2019. Arabidopsis ITPK1 and ITPK2 Have an Evolutionarily Conserved Phytic Acid Kinase Activity. ACS Chem. Biol. 14(10):2127-33
Laha N P, Dhir Y W, Giehl R F H, Schäfer E M, Gaugler P, et al. 2020. ITPK1-Dependent Inositol Polyphosphates Regulate Auxin Responses in Arabidopsis thaliana. bioRxiv. 2020.04.23.058487
Land E S, Cridland C A, Craige B, Dye A, Hildreth S B, et al. 2021. Metabolic Adaptations to Low Phosphate in Plants; A Role for Inositol Pyrophosphates. 1-23
Lin W Y, Huang T K, Chiou T J. 2013. Nitrogen limitation adaptation, a target of microRNA827, mediates degradation of plasma membrane-localized phosphate transporters to maintain phosphate homeostasis in Arabidopsis. Plant Cell. 25(10):4061-74
Liu T Y, Huang T K, Tseng C Y, Lai Y S, Lin S I, et al. 2012. PHO2-dependent degradation of PHO1 modulates phosphate homeostasis in Arabidopsis. Plant Cell. 24(5):2168-83
Liu T Y, Huang T K, Yang S Y, Hong Y T, Huang S M, et al. 2016. Identification of plant vacuolar transporters mediating phosphate storage. Nat. Commun. 7(1):11095
Losito O, Szijgyarto Z, Resnick A C, Saiardi A. 2009. Inositol pyrophosphates and their unique metabolic complexity: analysis by gel electrophoresis. PLoS One. 4(5):
Luan M, Zhao F, Han X, Sun G, Yang Y, et al. 2019. Vacuolar Phosphate Transporters Contribute to Systemic Phosphate Homeostasis Vital for Reproductive Development in Arabidopsis. Plant Physiol. 179(2):640-55
Lv Q, Zhong Y, Wang Y, Wang Z, Zhang L, et al. 2014. SPX4 Negatively Regulates Phosphate Signaling and Homeostasis through Its Interaction with PHR2 in Rice. Plant Cell. 26(4):1586-97
Mason M G, Ross J J, Babst B A, Wienclaw B N, Beveridge C A. 2014. Sugar demand, not auxin, is the initial regulator of apical dominance. Proc. Natl. Acad. Sci. U.S.A 111(16):6092-97
Mosblech A, Thurow C, Gatz C, Feussner I, Heilmann I. 2011. Jasmonic acid perception by COI1 involves inositol polyphosphates in Arabidopsis thaliana. Plant J. 65(6):949-57
Mulugu S, Bai W, Fridy P C, Bastidas R J, Otto J C, et al. 2007. A conserved family of enzymes that phosphorylate inositol hexakisphosphate. Science (80-.). 316(5821):106-9
Nakamura Y. 2013. Phosphate starvation and membrane lipid remodeling in seed plants
Osorio M B, Ng S, Berkowitz O, De Clercq I, Mao C, et al. 2019. SPX4 Acts on PHR1-Dependent and -Independent Regulation of Shoot Phosphorus Status in Arabidopsis. Plant Physiol. 181(1):332
Park B S, Yao T, Seo J S, Wong E C C, Mitsuda N, et al. 2018. Arabidopsis NITROGEN LIMITATION ADAPTATION regulates ORE1 homeostasis during senescence induced by nitrogen deficiency. Nat. plants. 4(11):898-903
Pascual-Ortiz M, Saiardi A, Walla E, Jakopec V, Künzel N A, et al. 2018. Asp1 Bifunctional Activity Modulates Spindle Function via Controlling Cellular Inositol Pyrophosphate Levels in Schizosaccharomyces pombe. Mol. Cell. Biol. 38(9):e00047-18
Peng M, Hannam C, Gu H, Bi Y M, Rothstein S J. 2007. A mutation in NLA, which encodes a RING-type ubiquitin ligase, disrupts the adaptability of Arabidopsis to nitrogen limitation. Plant J. 50(2):320-37
Péret B, Clément M, Nussaume L, Desnos T. 2011. Root developmental adaptation to phosphate starvation: better safe than sorry. Trends Plant Sci. 16(8):442-50
Pöhlmann J, Risse C, Seidel C, Pohlmann T, Jakopec V, et al. 2014. The Vip1 Inositol Polyphosphate Kinase Family Regulates Polarized Growth and Modulates the Microtubule Cytoskeleton in Fungi. PLoS Genet. 10(9):e1004586
Poirier Y, Thoma S, Somerville C, Schiefelbein J. 1991. Mutant of Arabidopsis deficient in xylem loading of phosphate. Plant Physiol. 97(3):1087-93
Puga M I, Mateos I, Charukesi R, Wang Z, Franco-Zorrilla J M, et al. 2014. SPX1 is a phosphate-dependent inhibitor of Phosphate Starvation Response 1 in Arabidopsis. Proc. Natl. Acad. Sci. U.S.A 111(41):14947-52
Raboy V. 2007. The ABCs of low-phytate crops
Ribot C, Wang Y, Poirier Y. 2008a. Expression analyses of three members of the AtPHO1 family reveal differential interactions between signaling pathways involved in phosphate deficiency and the responses to auxin, cytokinin, and abscisic acid. Planta. 227(5):1025-36
Ribot C, Zimmerli C, Farmer E E, Reymond P, Poirier Y. 2008b. Induction of the Arabidopsis PHO1;H10 gene by 12-oxo-phytodienoic acid but not jasmonic acid via a CORONATINE INSENSITIVE1-dependent pathway. Plant Physiol. 147(2):696-706
Rouached H, Stefanovic A, Secco D, Bulak Arpat A, Gout E, et al. 2011. Uncoupling phosphate deficiency from its major effects on growth and transcriptome via PHO1 expression in Arabidopsis. Plant J. 65(4):557-70
Ruan W, Guo M, Wang X, Guo Z, Xu Z, et al. 2019. Two RING-Finger Ubiquitin E3 Ligases Regulate the Degradation of SPX4, An Internal Phosphate Sensor, for Phosphate Homeostasis and Signaling in Rice. Mol. Plant. 12(8):1060-74
Rubio V. 2001. A conserved MYB transcription factor involved in phosphate starvation signaling both in vascular plants and in unicellular algae. Genes Dev. 15(16):2122-33
Secco D, Wang C, Arpat B A, Wang Z, Poirier Y, et al. 2012. The emerging importance of the SPX domain-containing proteins in phosphate homeostasis
Sheard L B, Tan X, Mao H, Withers J, Ben-Nissan G, et al. 2010. Jasmonate perception by inositol-phosphate-potentiated COI1-JAZ co-receptor. Nature. 468(7322):400-407
Stefanovic A, Ribot C, Rouached H, Wang Y, Chong J, et al. 2007. Members of the PHO1 gene family show limited functional redundancy in phosphate transfer to the shoot, and are regulated by phosphate deficiency via distinct pathways. Plant J. 50(6):982-94
Stevenson-Paulik J, Bastidas R J, Chiou S T, Frye R A, York J D. 2005. Generation of phytate-free seeds in Arabidopsis through disruption of inositol polyphosphate kinases. Proc. Natl. Acad. Sci. U.S.A 102(35):12612-17
Sweetman D, Stavridou I, Johnson S, Green P, Caddick S E K, Brearley C A. 2007. Arabidopsis thaliana inositol 1,3,4-trisphosphate 5/6-kinase 4 (AtITPK4) is an outlier to a family of ATP-grasp fold proteins from Arabidopsis. FEBS Lett. 581(22):4165-71
Tan X, Calderon-Villalobos L I A, Sharon M, Zheng C, Robinson C V, et al. 2007. Mechanism of auxin perception by the TIR1 ubiquitin ligase. Nature. 446(7136):640-45
Verbsky J W, Wilson M P, Kisseleva M V., Majerus P W, Wente S R. 2002. The synthesis of inositol hexakisphosphate: Characterization of human inositol 1,3,4,5,6-pentakisphosphate 2-kinase. J. Biol. Chem. 277(35):31857-62
Wang H, Falck J R, Hall T M T, Shears S B. 2012. Structural basis for an inositol pyrophosphate kinase surmounting phosphate crowding. Nat. Chem. Biol. 8(1):111-16
Wang Y, Ribot C, Rezzonico E, Poirier Y. 2004. Structure and expression profile of the Arabidopsis PHO1 gene family indicates a broad role in inorganic phosphate homeostasis. Plant Physiol. 135(1):400-411
Wang Z, Kuo H F, Chiou T J. 2021. Intracellular phosphate sensing and regulation of phosphate transport systems in plants. Plant Physiol. 187(4):2043
Wang Z, Ruan W, Shi J, Zhang L, Xiang D, et al. 2014. Rice SPX1 and SPX2 inhibit phosphate starvation responses through interacting with PHR2 in a phosphate-dependent manner. Proc. Natl. Acad. Sci. U.S.A 111(41):14953-58
Wild R, Gerasimaite R, Jung J-Y, Truffault V, Pavlovic I, et al. 2016. Control of eukaryotic phosphate homeostasis by inositol polyphosphate sensor domains. Science (80-.). 352(6288):986-90
Wilson M S C, Bulley S J, Pisani F, Irvine R F, Saiardi A. 2015. A novel method for the purification of inositol phosphates from biological samples reveals that no phytate is present in human plasma or urine. Open Biol. 5(3):
Yaeno T, Iba K. 2008. BAH1/NLA, a RING-type ubiquitin E3 ligase, regulates the accumulation of salicylic acid and immune responses to Pseudomonas syringae DC3000. Plant Physiol. 148(2):1032-41
Yan H, Sheng M, Wang C, Liu Y, Yang J, et al. 2019. AtSPX1-mediated transcriptional regulation during leaf senescence in Arabidopsis thaliana. Plant Sci. 283:238-46
Zhang L. 2017. Identification and Characterization of Genes underlying Natural Variation in Flowering Time in Arabidopsis thaliana
Zhou Y, Ni M. 2009. SHB1 plays dual roles in photoperiodic and autonomous flowering. Dev. Biol. 331(1):50-57
Zhou Y, Zhang X, Kang X, Zhao X, Zhang X, Ni M. 2009. SHORT HYPOCOTYL UNDER BLUE1 associates with MINISEED3 and HAIKU2 promoters in vivo to regulate Arabidopsis seed development. Plant Cell. 21(1):106-17
Zhou Z, Wang Z, Lv Q, Shi J, Zhong Y, et al. 2015. SPX proteins regulate Pi homeostasis and signaling in different subcellular level. Plant Signal. Behav. 10(9):e1061163
Zhu J, Lau K, Puschmann R, Harmel R K, Zhang Y, et al. 2019. Two bifunctional inositol pyrophosphate kinases/phosphatases control plant phosphate homeostasis. Elife. 8:1-25

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. An engineered plant comprising:
increased inositol pyrophosphates and/or synthesis thereof as compared to a wild-type or unmodified plant.

2. The engineered plant of aspect 1, wherein the engineered plant comprises increased expression and/or amount of
a. an inositol tetrakisphosphate kinase (ITPK) gene and/or gene product;
b a diphosphoinositol pentakisphosphate (VIP) gene and/or gene product; or
c. both (a) and (b).

3. The engineered plant of aspect 2, wherein the ITPK gene and/or gene product is an inositol tetrakisphosphate 1-kinase 1 (ITPK1) gene and/or gene product or an inositol tetrakisphosphate kinase 2 (ITPK2) gene and/or gene product.

4. The engineered plant of any one of aspects 2-3, wherein the VIP gene and/or gene product is a inositol diphosphoinositol pentakisphosphate 1 (VIP1) gene and/or gene product or an inositol diphosphoinositol pentakisphosphate 2 (VIP2) gene and/or gene product.

5. The engineered plant of any one of aspects 2-4, wherein (a) the VIP gene and/or gene product comprises or consists of the kinase domain of dual domain diphosphoinositol pentakisphosphate kinase 1 (VIP1KD) and/or the kinase domain of the dual domain diphosphoinositol pentakisphosphate kinase 2 (VIP2KD), (b) the ITPK gene and/or gene product comprises or consists of the kinase domain of the inositol tetrakisphosphate 1-kinase 1 (ITPK1KD) and/or the kinase domain of the inositol tetrakisphosphate kinase 2 (ITPK2KD).

6. The engineered plant of any one of aspects 2-5, wherein the increase in expression and/or amount of ITPK1 gene and/or gene product, VIP gene and/or gene product, and/or the one or more gene and/or gene products thereof associated with the phosphate starvation response is 1-1,000 fold or more as compared to a wild-type or unmodified plant.

7. The engineered plant of any one of aspects 1-6, wherein the engineered plant has increased phosphorous use efficiency as compared to a wild-type or unmodified plant.

8. The engineered plant of any one of aspects 1-7, wherein phosphorous use efficiency is increased 1-1,000 fold or more as compared to a wild-type or unmodified plant.

9. The engineered plant of any one of aspects 1-8, wherein the engineered plant has reduced Pi accumulation capability and/or capacity as compared to a wild-type or unmodified plant.

10. The engineered plant of aspect 9, wherein the Pi accumulation capability and/or capacity is reduced by 1-1,000 fold or more as compared to a wild-type or unmodified plant.

11. The engineered plant of any one of aspects 1-10, wherein the engineered plant has one or more modified developmental pathways and/or hormone signaling pathways.

12. The engineered plant of any one of aspects 1-11, wherein the engineered plant comprises one or more modifications to
a. an endogenous ITPK1 gene and/or gene product;
b an endogenous VIP gene and/or gene product; or
c. both (a) and (b),
wherein the one or more modifications increases the expression of an ITPK gene and/or gene product and/or a VIP gene and/or gene product as compared to a wild-type or unmodified plant or cell(s) thereof.

13. The engineered plant of aspect 13, wherein the one or more modifications comprise an insertion of one or more nucleotides, a deletion of one or more nucleotides, a substitution of one or more nucleotides, or any combination thereof in the endogenous ITPK gene and/or gene product and/or endogenous VIP gene and/or gene product.

14. A method of producing an engineered plant and/or increasing phosphorus use efficiency in a plant comprising:
overexpressing, in a plant and/or one or more plant cells
a. an inositol tetrakisphosphate kinase (ITPK) gene and/or gene product;
b a diphosphoinositol pentakisphosphate (VIP) gene and/or gene product; or
c. both (a) and (b).

15. The method of aspect 14, wherein the ITPK gene and/or gene product is an inositol tetrakisphosphate 1-kinase 1 (ITPK1) gene and/or gene product or an inositol tetrakisphosphate kinase 2 (ITPK2) gene and/or gene product.

16. The method of any one of aspects 14-15, wherein the VIP gene and/or gene product is a inositol diphosphoinositol pentakisphosphate 1 (VIP1) gene and/or gene product or an inositol diphosphoinositol pentakisphosphate 2 (VIP2) gene and/or gene product

17. The method of any one of aspects 14-16, wherein overexpressing comprises introducing, into one or more cells of a plant,
a. an exogenous ITPK gene and/or gene product;
b. an exogenous VIP gene and/or gene product; or
c. both (a) and (b).

18. The method of any one of aspects 14-17, wherein overexpression comprises introducing, into one or more cells of a plant, one or more modifications in
a. an endogenous ITPK gene and/or gene product;
b. an endogenous VIP gene and/or gene product; or
c. both (a) and (b),
wherein the one or more modifications increases the expression of a ITPK1 gene and/or gene product, a VIP gene and/or gene product, one or more gene and/or gene products associated with the phosphate starvation response, or any combination thereof as compared to a wild-type or unmodified plant or cell(s) thereof.

19. The method of aspect 18, wherein the one or more modifications comprise an insertion of one or more nucleotides, a deletion of one or more nucleotides, a substitution of one or more nucleotides, or any combination thereof in the endogenous ITPK gene and/or gene product and/or endogenous VIP gene and/or gene product.

20. A method comprising:
planting, growing, harvesting, and/or cultivating an engineered plant of any one of aspects 1-13 or made by a method of any one of aspects 14-19 in a growth medium, wherein the growth medium has low phosphorous, low phosphorus availability, or both.

21. The method of aspect 20, wherein the growth medium has low Pi, low Pi availability, or both.

22. The method of any one of aspects 20-21, wherein the growth medium is soil or an aqueous environment.

23. The method of any one of aspects 20-22, wherein planting, growing, harvesting, and/or cultivating comprises a reduced or eliminated application of an amount supplemental phosphorous and/or Pi as compared to planting, growing, harvesting, and/or cultivating a suitable control plant and/or suitable control planting, growing, harvesting, and/or cultivating conditions.

1. An engineered plant comprising:
increased inositol pyrophosphates and/or synthesis thereof as compared to a wild-type or unmodified plant.

2. The engineered plant of claim 1, wherein the engineered plant comprises increased expression and/or amount of
a. an inositol tetrakisphosphate kinase (ITPK) gene and/or gene product;
b. a diphosphoinositol pentakisphosphate (VIP) gene and/or gene product; or
c. both (a) and (b).

3. The engineered plant of claim 2, wherein the ITPK gene and/or gene product is an inositol tetrakisphosphate 1-kinase 1 (ITPK1) gene and/or gene product or an inositol tetrakisphosphate kinase 2 (ITPK2) gene and/or gene product.

4. The engineered plant of claim 2, wherein the VIP gene and/or gene product is an inositol diphosphoinositol pentakisphosphate 1 (VIP1) gene and/or gene product or an inositol diphosphoinositol pentakisphosphate 2 (VIP2) gene and/or gene product.

5. The engineered plant of claim 2, wherein (a) the VIP gene and/or gene product comprises the kinase domain of dual domain diphosphoinositol pentakisphosphate kinase 1 (VIP1KD) and/or the kinase domain of the dual domain diphosphoinositol pentakisphosphate kinase 2 (VIP2KD), (b) the ITPK gene and/or gene product comprises the kinase domain of the inositol tetrakisphosphate 1-kinase 1 (ITPK1KD) and/or the kinase domain of the inositol tetrakisphosphate kinase 2 (ITPK2KD), or both (a) and (b).

6. The engineered plant of claim 2, wherein the increase in expression and/or amount of ITPK1 gene and/or gene product, VIP gene and/or gene product, and/or the one or more gene and/or gene products thereof associated with the phosphate starvation response is 1-1,000 fold or more as compared to a wild-type or unmodified plant.

7. The engineered plant of claim 1, wherein
(a) the engineered plant has increased phosphorous use efficiency as compared to a wild-type or unmodified plant;
(b) phosphorous use efficiency is increased 1-1,000 fold or more as compared to a wild-type or unmodified plant;
(c) the engineered plant has reduced Pi accumulation capability and/or capacity as compared to a wild-type or unmodified plant;
(d) or any combination of (a)-(c).

8. (canceled)

9. (canceled)

10. The engineered plant of claim 7, wherein the Pi accumulation capability and/or capacity is reduced by 1-1,000 fold or more as compared to a wild-type or unmodified plant.

11. The engineered plant of claim 1, wherein the engineered plant has one or more modified developmental pathways and/or hormone signaling pathways.

12. The engineered plant of claim 1, wherein the engineered plant comprises one or more modifications to
a. an endogenous ITPK1 gene and/or gene product;
b. an endogenous VIP gene and/or gene product; or
c. both (a) and (b),
wherein the one or more modifications increases the expression of an ITPK gene and/or gene product and/or a VIP gene and/or gene product as compared to a wild-type or unmodified plant or cell(s) thereof, and wherein the one or more modifications comprise an insertion of one or more nucleotides, a deletion of one or more nucleotides, a substitution of one or more nucleotides, or any combination thereof in the endogenous ITPK gene and/or gene product and/or endogenous VIP gene and/or gene product.

13. (canceled)

14. A method of producing an engineered plant and/or increasing phosphorus use efficiency in a plant comprising:
overexpressing, in a plant and/or one or more plant cells
a. an inositol tetrakisphosphate kinase (ITPK) gene and/or gene product;
b. a diphosphoinositol pentakisphosphate (VIP) gene and/or gene product; or
c. both (a) and (b).

15. The method of claim 14, wherein the ITPK gene and/or gene product is an inositol tetrakisphosphate 1-kinase 1 (ITPK1) gene and/or gene product or an inositol tetrakisphosphate kinase 2 (ITPK2) gene and/or gene product.

16. The method of claim 14, wherein the VIP gene and/or gene product is an inositol diphosphoinositol pentakisphosphate 1 (VIP1) gene and/or gene product or an inositol diphosphoinositol pentakisphosphate 2 (VIP2) gene and/or gene product

17. The method of claim 14, wherein overexpressing comprises introducing, into one or more cells of a plant,
a. an exogenous ITPK gene and/or gene product;
b. an exogenous VIP gene and/or gene product; or
c. both (a) and (b).

18. The method of claim 14, wherein overexpression comprises introducing, into one or more cells of a plant, one or more modifications in
a. an endogenous ITPK gene and/or gene product;
b. an endogenous VIP gene and/or gene product; or
c. both (a) and (b),
wherein the one or more modifications increases the expression of a ITPK1 gene and/or gene product, a VIP gene and/or gene product, one or more gene and/or gene products associated with the phosphate starvation response, or any combination thereof as compared to a wild-type or unmodified plant or cell(s) thereof, wherein the one or more modifications comprise an insertion of one or more nucleotides, a deletion of one or more nucleotides, a substitution of one or more nucleotides, or any combination thereof in the endogenous ITPK gene and/or gene product and/or endogenous VIP gene and/or gene product.

19. The method of claim 15, wherein (a) the VIP gene and/or gene product comprises the kinase domain of dual domain diphosphoinositol pentakisphosphate kinase 1 (VIP1KD) and/or the kinase domain of the dual domain diphosphoinositol pentakisphosphate kinase 2 (VIP2KD), (b) the ITPK gene and/or gene product comprises the kinase domain of the inositol tetrakisphosphate 1-kinase 1 (ITPK1KD) and/or the kinase domain of the inositol tetrakisphosphate kinase 2 (ITPK2KD), or both (a) and (b).

20. A method comprising:
planting, growing, harvesting, and/or cultivating an engineered plant of claim 1 in a growth medium, wherein the growth medium has low phosphorous, low phosphorus availability, or both.

21. The method of claim 20, wherein the growth medium has low Pi, low Pi availability, or both.

22. The method of claim 20, wherein the growth medium is soil or an aqueous environment.

23. The method of claim 20, wherein planting, growing, harvesting, and/or cultivating comprises a reduced or eliminated application of an amount supplemental phosphorous and/or Pi as compared to planting, growing, harvesting, and/or cultivating a suitable control plant and/or suitable control planting, growing, harvesting, and/or cultivating conditions.


SEQUENCE LISTING (26 sequences)

SEQ ID NO: 1 - RNA, 1698 nt, Arabidopsis thaliana
  source 1..1698: mol_type = mRNA
gtacaacattcccttctctctctgttatttcatctctagaaaagggcacaaaaaaccaaactttacgatttctcctctctggaatctcgattctgtatgatctcttcgatctaatcgagtcttctaaacgttccgtgtagaatttcaaatctagggtttgctaaagcttgaacctttcttacccttttctctcgaagaggttttgattcctgggcatgaatttttccctaatcttgttaccttcgaagcataacaacaacgaaagatgtcagattcaatccaggaaagatacttagttggatacgcactcgcagccaagaaacagcatagtttcatccaaccttctttgatcgaacactcaaggcaacgaggcattgatctggtcaagcttgatccgacgaaatcgttgttggagcaagggaagcttgattgcataatccacaagctttacgatgtgtattggaaggagaatcttcatgagtttcgcgagaaatgtcctggtgttcctgtaatcgatttgcctgaggctatcgagcgattgcacaatagggtttcgatgcttgaggtgataactcaattgaggtttcctgtttcagatagcgagagatttggtgtcccggagcaagttgttgttatggattcgagcgtcttgagcggtggaggagctttaggggagcttaagtttccggtgattgctaagccgttggatgctgatgggagtgctaaatctcataagatgtttttgatttatgatcaagaagggatgaagattttgaaagctcctattgtgttgcaggagtttgtgaatcatggtggtgtgatctttaaggtctatgtggttggagatcatgtcaaatgtgtaaagagaagatctttacctgatatctctgaagagaagattgggacgtcgaaagggtctcttccgttttcgcagatatcgaatctgactgctcaagaggacaagaacattgagtatggtgaggataggagcttagagaaagtggaaatgcctccgttaagtttcttgacagatctagcaaaggccatgagggaatcaatgggactcaatctctttaacttcgacgtgattagggatgccaaagatgctaataggtaccttataattgatattaactactttcctggatatgctaagatgccctcttacgagcctgtgttgactgagttcttctgggacatggtcactaagaagaatcatgtctgagaaataatctgttggggagttagcaggaacattgttggggaggatttgtaccattgcaatgtgttgcaactgccattgcaattctggcctatggtgactagctgctttgctaattttcatttggattcttgaacttgtgctttttatatttttttagttgtttctcttttgtttacttccgagttttgcaaagccatgttcaaatgcataactgttgctactcttgaaaagcattgaatctctaaattcttggcaatgtggtctgttctcaactgttggtagtttgtgatttcttttcaagcttttatggtgcctgttttcttcctcaaatctgacaagactgagatatttgattaagcattggtataagttgattagtaaccttcacttggcaatgcttttccattctctatattatgaactgtttatgtgaaaccggtgctaagtctttgtatcggatttctaagacatgg

SEQ ID NO: 2 - RNA, 1786 nt, Arabidopsis thaliana
  source 1..1786: mol_type = mRNA
cccaagcttgaacttcttgctaggtaaccaataacaaacccaaaatgcttctttggagttataaagttttgatttttcattgcaaagtttccaaatttgttgaacaatcttttttggttctgctttttctttcctgagagacgactgttcctttgttggcgtttaaacataaccgacaaactcttttctactttcaaaaagatctttaggactttgacattttcttccagatttggttcttgtttccttactcttgttttggtacctattattggtttgcctgattctatgaggaccattgccacttgccatgtcttgtcacctgccatgtttttgtccttaatcggctcttttgcttagggttaggcttacactagtatactctacttgtggtatcaggatgtttctatgggaatattccttgtgtaatccttttggattagtctctattgaggtttgactctggcttttaaaaaataaaatacttagatgttttgccttttctgtggcttggaaatcttcttcaaccacatgggtgtttttgttaaagcaatcactggttctacttaatatgtcatcttctagaatgtctctaataggtcagagagaacatttcttattggtttagtttgacgtctggtctctgttttgcttctttctaatcaacccatagctttcattctttttcttctttcagcagtgttttcaagaaaatgctagtaatactttttgtgtctgtcaatttcaggagaaaaggcattttctttgtcgccatcgatttgaaccggcccctatctgaacaagggccatttgatgttgttttgcataagttgttgggaaaagagtgggaagaggttattgaggattaccaacaaaaacacccagaagtgactgtgcttgatcctccaggatcaatacagcgtatatataatcgacaatcgatgcttcagggtatggcagatttgaaactgtcagattgcagtggcagcctttttgttccaaagcaaatggttgtcttgaaagattcagcagctagtgctgatgcagttgtggaagctggtctcaaatttccactagttgcaaagccgctctggatcgatgggactgcaaagtcacatcaattgtacttggcttatgacaggcgctcgcttgcagagcttgatccgcctttagtccttcaagagtttgttaatcatggtggagttatgttcaaggtatttgtggtgggtgatgttataaaagtcatgagacggttttctctaccaaatgtgagtaattgtgaaaaagccaaagttgatggcgtcttccaattcccaagggtttcatcagctgctgcttcagctgataacgcagacttggaccctcgtgttgctgagctacctccaaagcctttcctcgaggcgcttgtgaaagagctaagaagcttattgggacttcggcttttcaacatagacatgatcagggaacatgggagcaaaaacgtgttttatgttattgacatcaactattttcctggttacggaaaactgccagactacgagcaagtctttgtagatttcttccaaaatctggcgcaggtcaaatataagaagagacaacattgtaaatgaaagaaaatggcggcagtttttagatggtctactaagaagcgacaaataataaaatgtctaattatggatttgtacagaatttagctctcccttttggaagcagtatcagataaacaagttttgattactgattttgaattttcagtgaaataaacgtctcttcatccaaagttgac

SEQ ID NO: 3 - protein, 319 aa, Arabidopsis thaliana
MSDSIQERYLVGYALAAKKQHSFIQPSLIEHSRQRGIDLVKLDPTKSLLEQGKLDCIIHKLYDVYWKENLHEFREKCPGVPVIDLPEAIERLHNRVSMLEVITQLRFPVSDSERFGVPEQVVVMDSSVLSGGGALGELKFPVIAKPLDADGSAKSHKMFLIYDQEGMKILKAPIVLQEFVNHGGVIFKVYVVGDHVKCVKRRSLPDISEEKIGTSKGSLPFSQISNLTAQEDKNIEYGEDRSLEKVEMPPLSFLTDLAKAMRESMGLNLFNFDVIRDAKDANRYLIIDINYFPGYAKMPSYEPVLTEFFWDMVTKKNHV

SEQ ID NO: 4 - protein, 391 aa, Arabidopsis thaliana
MFGTLASGEIETARLNRNLGITSNLGVSCGGFEDFAMRFEGENMVPYKGEEQEEEEDQVVVNETTPFQFQQPLFLQQQQKLVVGYALTSKKKKSFLQPKLELLARRKGIFFVAIDLNRPLSEQGPFDVVLHKLLGKEWEEVIEDYQQKHPEVTVLDPPGSIQRIYNRQSMLQGMADLKLSDCSGSLFVPKQMVVLKDSAASADAVVEAGLKFPLVAKPLWIDGTAKSHQLYLAYDRRSLAELDPPLVLQEFVNHGGVMFKVFVVGDVIKVMRRFSLPNVSNCEKAKVDGVFQFPRVSSAAASADNADLDPRVAELPPKPFLEALVKELRSLLGLRLFNIDMIREHGSKNVFYVIDINYFPGYGKLPDYEQVFVDFFQNLAQVKYKKRQHCK

SEQ ID NO: 5 - RNA, 3561 nt, Arabidopsis thaliana
  source 1..3561: mol_type = mRNA
aattctgattcaaagcaacgcctctgacgcctgaatcttctggctctctctctttcctcgtgccatcagcgcactatctctagctattgctctctctgcttgtgatcatcattttccccaacggagaacgagagaacgtcgttcgaggaacgaaacggaaagaggttcgtatcgggaggagagagcgaaaatgggggtggaagaaggagctggtgttgataagaagataacgattggagtctgcgtcatggaaaagaaggttttttcagctcccatgggacaaattatggacaggatacatgcgtttggcgaatttgagattatacattttggggacaaggtcatacttgaagacccagtagaaagttggccgatttgtgattgtttgattgctttctattcctctggatatcctcttgagaaagttcaagcatattcttctttaagaaagccctttttagtgaatgaactcgatccccaatatcttcttcatgaccgccggaaagtgtatgagcatcttgagatgtatggtatcccagttcctagatatgcttgtgtcaatagaaaagtaccagacgaagatcttgattatttcgtcgaggaagaagattttgttgaggttaaaggtgaacgattctggaagccatttgtggaaaagcccgtcaatggagatgaccatagtataatgatatactaccctagctcagctggtggaggcatgaaagaattgtttcgtaaggttgggaatcgatcaagtgagtttcatcctgatgtcagaagagtaaggagagaaggttcttatatatatgaggagtttatgcctactgggggaactgatgtcaaggtctacactgtgggtcccgaatatgcacacgctgaagcaagaaaatcacctgttgttgatggtgtagtaatgagaaatccagatgggaaggaagtgagatatccagttttacttacacctgctgagaaacaaatggcgagagaagtttgcattgcatttaggcaagcggtttgcggttttgatctcctacgatctgagggaagttcatatgtttgtgacgtaaatggatggagttttgtgaagaactcctataagtattacgacgatgctgcttgtgtgctacggaaaatgtttttggatgcaaaggctcctcatctttcttcaacaattcctcccattctgccatggaaaatcaacgaacctgtccaatctaatgaaggtcttacccgccaaggaagtggcatcatcggaacttttgggcagtcagaggagctacgctgtgtcattgctattgttcggcatggtgatagaacccccaagcagaaagtgaaactaaaagttacagaggagaaactgttaaacctgatgttgaagtacaatggaggaaaaccaagagctgagacaaaacttaaaactgccgtccaattgcaagatctattggatgccacaagaatgttaattcctcgtgcaagatcaggtgaaagtgatagtgatgcagaagaccttgaacacgctgacaagcttcggcaagttaaagccgttcttgaagagggtgggcatttctctggcatatacaggaaggttcaactgaagccgttgaaatgggttaacgtaccaaaaagtgatggtgaaggcgaagaggaacgcccagtggaagctcttatggttctgaaatacgggggtgttctaactcatgctggtagaaagcaggccgaagagcttggtagatactttcgaaacaatatgtatccaggggaaggtaccggtttgctccgtctccatagtacataccgtcacgaccttaaaatttacagctctgacgagggacgtgttcagatgtctgcagctgcttttgccaaaggcctgcttgacctagaaggacagctgaccccaatcctggtttctctggtaagtaaggactcttccatgttggatggccttgataatgccagcagtgaaatggaagcagctaaggctcaactgaatgagatcataaccgccggctcaaagatggtacatgatcacgtctcttctgaattaccttggatgactgatggggctggacttcctcctcacgctgatgagcacctacctgaattggtaaaattagctaaaaaggtgactgaacaagtaaggctacttgcacaagatgaacacgagaatctcgctgagcctagcgcttatgatgtagtccctccatatgatcaagccaaggcccttggcaagtcaaacattgacgttggtcggattgctgccggattaccttgtggtagtgaagggttccttttgatgtttgcccggtggagaaaactcgaaagggatctctacaacgaaagaagagagcggtttgacattacgcagattcctgatgtttacgattcatgcaagtatgacctgttacataattctcatctcgatctaaaaggactagacgaactcttcaaagtagcgcagttgcttgcagatggtgtaatcccaaatgagtatgggatcaatccacagcaaaagctcaaaatcggttcaaagattgcacggcgcctgctgggtaaaatccttattgacttgaggaacactcgagaagaggccatgagtgttgctgaattgaagaacagtcaagatcaagtctcagtgtcattgtattcgtcaagaaaggaggatagatatagtcaaccaaaacttttcgttaaaagcgatgagttgagacggcctagcactggagagaataaagaagaagatgatgataaagaaaccaaatacagactagatccaaagtatgcaaatgtcatgacgcctgaacgtcatgtgaggacacgtctttacttcacatctgaatcacatattcactctctgatgaacgttctacggtattgtaatcttgacgaatctcttcaaggagaagaaagtctggtctgccaaagcgcgttggatcgtctatgtaaaaccaaagaacttgattacatgagctacgttgtcctaaggctgtttgagaacactgagatatctctggacgacccgaaacgatttagaatcgaacttacatttagccgtggcgcagatttgtctcccttagagaagaaggacgaggaagcagaatcattgcttcgagaacacacacttccgataatgggaccagaaaggcttcaagaggttggttcatgtttgaccctcgagacaatggagaagatgatacgtccatttgcaatgccagccgaggatttccctccaccgtgtactccagcaggcttctccggttacttctctaaaagcgccgctgttctcgaacgtcttgttaaactttggcccttccacaagaacacctctaatggcaaaagctaaacgccaacaaagtgggtgactaatgactaatgaatgatgatgatattcctaatccatttattttttgaatcaaaattttgaattgttagtagtttctaattgagtacactaaattgttcattttgttttgtagaatcgtcaaaaaaaattgttaaaaacaattgctaattgatttagcattatggaaaatttccaagcttctacttgtcgtttgctttgat

SEQ ID NO: 6 - RNA, 3736 nt, Arabidopsis thaliana
  source 1..3736: mol_type = mRNA
cctgtgacgactgaattgtcctctgtctggccccgtgagcttcaccgcagcttttagatattttctttccccgttctttcggattttttttttctccccctctcaagcaaagcattatcgctattgctctgccttgtgagctgcgatcacctgtttactgagagaagaacactgcgacgtcgttaaaagtaggttgagagcgagaaaggccgagagaatggagatggaagaaggagcaagtggtgttggtgagaagataaagattggagtctgcgtcatggaaaagaaggtgaaatgcggctccgaggttttctcagctcccatgggggaaattctcgacagactccagtcttttggtgaatttgagatcttacattttggggataaagttatacttgaagatccaatagaaagttggcccatttgtgattgcttgattgcttttcattcctctggatatcctcttgagaaggctcaggcatatgctgctttaagaaagccatttttagtgaatgaacttgatccgcaatatcttcttcatgatcgccggaaggtgtatgagcatcttgagatgtatggcatcccagttcctaggtatgcttgtgtcaatagaaaggtaccaaatcaagaccttcattattttgtcgaggaagaagattttgttgaggtccatggtgaacgcttctggaagccatttgtggaaaagcctgtcaatggagatgaccatagtataatgatatactaccctagctcggcaggtggaggcatgaaagaattgtttcgcaagattgggaaccgatcaagtgaatttcatcctgacgtcagaagggtaaggagagaaggctcttatatatacgaggagtttatggccaccggaggaactgatgtcaaggtctatacagtgggtcctgaatacgcacatgctgaagcaagaaagtcacctgttgttgatggtgttgttatgaggaatacagatgggaaggaagtgagatatccagtattgcttacacctgctgaaaagcaaatggctagagaagtttgcattgcatttaggcaagcggtctgtgggtttgatcttctacgatctgagggctgttcatatgtttgtgatgtcaatggatggagttttgtgaagaactcatacaagtattatgacgatgctgcgtgtgtgctaaggaaaatgtgtttggatgcaaaagctcctcatctctcatccactcttcctcccaccttgccatggaaggtcaatgaacctgtacaatctaatgaaggtctaactcgccagggaagtggcatcatcggcacttttgggcagtcagaagagctacgttgtgtcattgctgttgttcgacatggcgatagaactcccaagcagaaagtgaaactaaaagttacagaggaaaaactattaaacctaatgttgaagtacaatggtggaaagccaagagctgagaccaaacttaagagtgccgtccagttgcaagatctattagatgccacaagaatgttagttccccgtacaagaccaggtcgtgaaagtgatagtgatgcagaagaccttgaacatgctgagaagcttcggcaagttaaagcagtccttgaagagggaggacatttctctggtatatacaggaaggttcaactgaagccgctgaagtgggttaaaataccaaaaagcgatggtgatggcgaagaagaaagaccagtagaggcccttatggttctgaaatatggcggtgttttaacacacgctggtagaaagcaggcagaagaacttggtagatactttcgaaacaatatgtatccaggtgaagggactggtttgcttcgtctccatagtacgtaccgtcatgaccttaaaatttacagctctgacgagggacgtgttcagatgtctgcagcggcttttgctaaaggcctgcttgacctagaaggacagctgacgccaatcctggtttctttggttagcaaggactcttccatgttggatggtcttgataatgccagcattgaaatggaagcggccaaggctagattgaatgagattgtaacgtctggcacaaagatgatagacgacgaccaagtctcctctgaagatttcccttggatgactgatggagctggacttcctcccaacgctcatgaactcctacgtgaactggtgaaattaactaagaatgtgactgaacaagtaagactacttgcaatggatgaagacgagaacctcactgagccatacgatataattcctccatatgatcaagcaaaagcccttggcaagacaaacattgacagtgatcggattgcttctggattaccatgtggtagtgaaggattccttctgatgtttgctcggtggataaaacttgctagggatctctacaatgaaagaaaagaccgatttgacatcacacagattccggatgtttacgattcatgcaagtacgacctgttacataattcccatctcgatctaaaaggattagatgaactcttcaaagtagcacagttacttgcagatggtgtaatcccaaatgagtatggcatcaatccgcaacaaaagcttaaaatcggttcaaagattgctcggcgcttaatggggaaaatcttgatagacttgaggaatactcgagaagaagcactgagcgttgccgagttgaaagaaagccaagaacaagtcttgtcattatccgcctcgcaaagggaagatagaaatagtcaaccgaagctatttatcaatagcgatgaattgagacgacctggcacaggtgataaagatgaagatgatgataaagaaaccaaataccgattagatccaaagtatgcaaatgtgaagacacctgaacgtcatgtgaggactcgactttacttcacatctgaatcgcatattcattcgctcatgaacgtccttcgatactgtaaccttgatgaatctctcctaggagaagaaagcctcatttgccaaaacgccttagaacgtctctgtaaaaccaaggaactcgattacatgagttacattgtcctaaggctctttgagaacaccgaggtatcacttgaagacccgaaaagattccgcattgaacttacatttagccgtggagctgatttgtctcccttaaggaataatgacgacgaggcagagacattgctaagggaacacacacttccgataatgggaccagagaggcttcaagaagtaggttcttgtttaagcttggagacaatggagaaaatggttcgtccatttgctatgccggcggaagatttccctccggcgtccactccggttggtttctcgggttacttctcaaaaagtgctgcggtgctggagcgactcgtaaacctcttccacaactataagaactcttcttctaatggaaggagctaagcagagaccaattgttttttccacttatatatattttgctcatgcttttttctatgtgcagtttttaatcatcttcttgtttttctactgatgttttaattttctatgtgaaattgtgaaagggactagtgcagttgggtttgaataaagatatgattacatggaacataaatgagtcacccgtgacgttgtctagtgtttacaataggactcatctattataactcaagaaatgatgatgagtcttattgtaaacactgtgttttcatatcatgctgttacgttaaagttgttgcggatttgttggaatttatatataatctgcaaaagaataggctgatgatatcg

SEQ ID NO: 7 - protein, 1049 aa, Arabidopsis thaliana
MGVEEGAGVDKKITIGVCVMEKKVFSAPMGQIMDRIHAFGEFEIIHFGDKVILEDPVESWPICDCLIAFYSSGYPLEKVQAYSSLRKPFLVNELDPQYLLHDRRKVYEHLEMYGIPVPRYACVNRKVPDEDLDYFVEEEDFVEVKGERFWKPFVEKPVNGDDHSIMIYYPSSAGGGMKELFRKVGNRSSEFHPDVRRVRREGSYIYEEFMPTGGTDVKVYTVGPEYAHAEARKSPVVDGVVMRNPDGKEVRYPVLLTPAEKQMAREVCIAFRQAVCGFDLLRSEGSSYVCDVNGWSFVKNSYKYYDDAACVLRKMFLDAKAPHLSSTIPPILPWKINEPVQSNEGLTRQGSGIIGTFGQSEELRCVIAIVRHGDRTPKQKVKLKVTEEKLLNLMLKYNGGKPRAETKLKTAVQLQDLLDATRMLIPRARSGESDSDAEDLEHADKLRQVKAVLEEGGHFSGIYRKVQLKPLKWVNVPKSDGEGEEERPVEALMVLKYGGVLTHAGRKQAEELGRYFRNNMYPGEGTGLLRLHSTYRHDLKIYSSDEGRVQMSAAAFAKGLLDLEGQLTPILVSLVSKDSSMLDGLDNASSEMEAAKAQLNEIITAGSKMVHDHVSSELPWMTDGAGLPPHADEHLPELVKLAKKVTEQVRLLAQDEHENLAEPSAYDVVPPYDQAKALGKSNIDVGRIAAGLPCGSEGFLLMFARWRKLERDLYNERRERFDITQIPDVYDSCKYDLLHNSHLDLKGLDELFKVAQLLADGVIPNEYGINPQQKLKIGSKIARRLLGKILIDLRNTREEAMSVAELKNSQDQVSVSLYSSRKEDRYSQPKLFVKSDELRRPSTGENKEEDDDKETKYRLDPKYANVMTPERHVRTRLYFTSESHIHSLMNVLRYCNLDESLQGEESLVCQSALDRLCKTKELDYMSYVVLRLFENTEISLDDPKRFRIELTFSRGADLSPLEKKDEEAESLLREHTLPIMGPERLQEVGSCLTLETMEKMIRPFAMPAEDFPPPCTPAGFSGYFSKSAAVLERLVKLWPFHKNTSNGKS

SEQ ID NO: 8 - protein, 1056 aa, Arabidopsis thaliana
MEMEEGASGVGEKIKIGVCVMEKKVKCGSEVFSAPMGEILDRLQSFGEFEILHFGDKVILEDPIESWPICDCLIAFHSSGYPLEKAQAYAALRKPFLVNELDPQYLLHDRRKVYEHLEMYGIPVPRYACVNRKVPNQDLHYFVEEEDFVEVHGERFWKPFVEKPVNGDDHSIMIYYPSSAGGGMKELFRKIGNRSSEFHPDVRRVRREGSYIYEEFMATGGTDVKVYTVGPEYAHAEARKSPVVDGVVMRNTDGKEVRYPVLLTPAEKQMAREVCIAFRQAVCGFDLLRSEGCSYVCDVNGWSFVKNSYKYYDDAACVLRKMCLDAKAPHLSSTLPPTLPWKVNEPVQSNEGLTRQGSGIIGTFGQSEELRCVIAVVRHGDRTPKQKVKLKVTEEKLLNLMLKYNGGKPRAETKLKSAVQLQDLLDATRMLVPRTRPGRESDSDAEDLEHAEKLRQVKAVLEEGGHFSGIYRKVQLKPLKWVKIPKSDGDGEEERPVEALMVLKYGGVLTHAGRKQAEELGRYFRNNMYPGEGTGLLRLHSTYRHDLKIYSSDEGRVQMSAAAFAKGLLDLEGQLTPILVSLVSKDSSMLDGLDNASIEMEAAKARLNEIVTSGTKMIDDDQVSSEDFPWMTDGAGLPPNAHELLRELVKLTKNVTEQVRLLAMDEDENLTEPYDIIPPYDQAKALGKTNIDSDRIASGLPCGSEGFLLMFARWIKLARDLYNERKDRFDITQIPDVYDSCKYDLLHNSHLDLKGLDELFKVAQLLADGVIPNEYGINPQQKLKIGSKIARRLMGKILIDLRNTREEALSVAELKESQEQVLSLSASQREDRNSQPKLFINSDELRRPGTGDKDEDDDKETKYRLDPKYANVKTPERHVRTRLYFTSESHIHSLMNVLRYCNLDESLLGEESLICQNALERLCKTKELDYMSYIVLRLFENTEVSLEDPKRFRIELTFSRGADLSPLRNNDDEAETLLREHTLPIMGPERLQEVGSCLSLETMEKMVRPFAMPAEDFPPASTPVGFSGYFSKSAAVLERLVNLFHNYKNSSSNGRS

SEQ ID NO: 9 - RNA, 1176 nt, Arabidopsis thaliana
  source 1..1176: mol_type = mRNA
atgtttgggactcttgcttccggcgaaatcgaaactgccagactgaaccgtaatttggggattacgtccaatttaggggtttcctgcggtggatttgaagactttgctatgagattcgaaggggaaaacatggtgccatacaaaggtgaagagcaggaggaggaagaagaccaagtggtggtgaacgaaacgacgccgttccagtttcaacaacctttgtttctgcagcagcagcagaagctcgttgttgggtatgctctgacttctaagaagaagaagagtttcttgcagcccaagcttgaacttcttgctaggagaaaaggcattttctttgtcgccatcgatttgaaccggcccctatctgaacaagggccatttgatgttgttttgcataagttgttgggaaaagagtgggaagaggttattgaggattaccaacaaaaacacccagaagtgactgtgcttgatcctccaggatcaatacagcgtatatataatcgacaatcgatgcttcagggtatggcagatttgaaactgtcagattgcagtggcagcctttttgttccaaagcaaatggttgtcttgaaagattcagcagctagtgctgatgcagttgtggaagctggtctcaaatttccactagttgcaaagccgctctggatcgatgggactgcaaagtcacatcaattgtacttggcttatgacaggcgctcgcttgcagagcttgatccgcctttagtccttcaagagtttgttaatcatggtggagttatgttcaaggtatttgtggtgggtgatgttataaaagtcatgagacggttttctctaccaaatgtgagtaattgtgaaaaagccaaagttgatggcgtcttccaattcccaagggtttcatcagctgctgcttcagctgataacgcagacttggaccctcgtgttgctgagctacctccaaagcctttcctcgaggcgcttgtgaaagagctaagaagcttattgggacttcggcttttcaacatagacatgatcagggaacatgggagcaaaaacgtgttttatgttattgacatcaactattttcctggttacggaaaactgccagactacgagcaagtctttgtagatttcttccaaaatctggcgcaggtcaaatataagaagagacaacattgtaaatga

SEQ ID NO: 10 - RNA, 1792 nt, Arabidopsis thaliana
  source 1..1792: mol_type = mRNA
tttcaagttgcttgtaatatggtttcatcttgagaaaaaaaaaaacagaaagagaaaatacaataaatgtattcagaaaagaagaataaccaaaaaaagcagcaaatctttgggaacgaccatgtcactgtagcatctgtttttctgcaaattattctctctataaatatatttgatcatcagaccaatctctccctcaacatttcctcatctcatctctctgatttcttgcttcgaattttgatttatctctctctgtctctctctcggtctcgtgctctatctcgtttgtaggcagtgccaatttcaccacttgtgttgtgtgtcagcggatatagaatctgagaggaatatttaaagattctccctttgcttcttcttttttataccgtttctattaatgtttgggactcttgcttccggcgaaatcgaaactgccagactgaaccgtaatttggggattacgtccaatttaggggtttcctgcggtggatttgaagactttgctatgagattcgaaggggaaaacatggtgccatacaaaggtgaagagcaggaggaggaagaagaccaagtggtggtgaacgaaacgacgccgttccagtttcaacaacctttgtttctgcagcagcagcagaagctcgttgttgggtatgctctgacttctaagaagaagaagagtttcttgcagcccaagcttgaacttcttgctaggagaaaaggcattttctttgtcgccatcgatttgaaccggcccctatctgaacaagggccatttgatgttgttttgcataagttgttgggaaaagagtgggaagaggttattgaggattaccaacaaaaacacccagaagtgactgtgcttgatcctccaggatcaatacagcgtatatataatcgacaatcgatgcttcagggtatggcagatttgaaactgtcagattgcagtggcagcctttttgttccaaagcaaatggttgtcttgaaagattcagcagctagtgctgatgcagttgtggaagctggtctcaaatttccactagttgcaaagccgctctggatcgatgggactgcaaagtcacatcaattgtacttggcttatgacaggcgctcgcttgcagagcttgatccgcctttagtccttcaagagtttgttaatcatggtggagttatgttcaaggtatttgtggtgggtgatgttataaaagtcatgagacggttttctctaccaaatgtgagtaattgtgaaaaagccaaagttgatggcgtcttccaattcccaagggtttcatcagctgctgcttcagctgataacgcagacttggaccctcgtgttgctgagctacctccaaagcctttcctcgaggcgcttgtgaaagagctaagaagcttattgggacttcggcttttcaacatagacatgatcagggaacatgggagcaaaaacgtgttttatgttattgacatcaactattttcctggttacggaaaactgccagactacgagcaagtctttgtagatttcttccaaaatctggcgcaggtcaaatataagaagagacaacattgtaaatgaaagaaaatggcggcagtttttagatggtctactaagaagcgacaaataataaaatgtctaattatggatttgtacagaatttagctctcccttttggaagcagtatcagataaacaagttttgattactgattttgaattttcagtgaaataaacgtctcttcatccaaagttgaccaaacgatatctgaattcttattttgaccatagcggttcg

SEQ ID NO: 11 - protein, 298 aa, Arabidopsis thaliana
MLVILFVSVNFRRKGIFFVAIDLNRPLSEQGPFDVVLHKLLGKEWEEVIEDYQQKHPEVTVLDPPGSIQRIYNRQSMLQGMADLKLSDCSGSLFVPKQMVVLKDSAASADAVVEAGLKFPLVAKPLWIDGTAKSHQLYLAYDRRSLAELDPPLVLQEFVNHGGVMFKVFVVGDVIKVMRRFSLPNVSNCEKAKVDGVFQFPRVSSAAASADNADLDPRVAELPPKPFLEALVKELRSLLGLRLFNIDMIREHGSKNVFYVIDINYFPGYGKLPDYEQVFVDFFQNLAQVKYKKRQHCK

SEQ ID NO: 12 - protein, 222 aa, Arabidopsis thaliana
MLQGMADLKLSDCSGSLFVPKQMVVLKDSAASADAVVEAGLKFPLVAKPLWIDGTAKSHQLYLAYDRRSLAELDPPLVLQEFVNHGGVMFKVFVVGDVIKVMRRFSLPNVSNCEKAKVDGVFQFPRVSSAAASADNADLDPRVAELPPKPFLEALVKELRSLLGLRLFNIDMIREHGSKNVFYVIDINYFPGYGKLPDYEQVFVDFFQNLAQVKYKKRQHCK

SEQ ID NO: 13 - DNA, 29 bp, Arabidopsis thaliana
  source 1..29: mol_type = other DNA
cttaactgcgactcagggaatcttctaag

SEQ ID NO: 14 - DNA, 24 bp, Arabidopsis thaliana
  source 1..24: mol_type = other DNA
tcatcctttcttaggcatagcggc

SEQ ID NO: 15 - DNA, 21 bp, Arabidopsis thaliana
  source 1..21: mol_type = other DNA
gattccattgttggagcaaga

SEQ ID NO: 16 - DNA, 24 bp, Arabidopsis thaliana
  source 1..24: mol_type = other DNA
aatctgttagcttcttctattgta

SEQ ID NO: 17 - DNA, 25 bp, Arabidopsis thaliana
  source 1..25: mol_type = other DNA
gaacggtcccaatagtttaggtgat

SEQ ID NO: 18 - DNA, 27 bp, Arabidopsis thaliana
  source 1..27: mol_type = other DNA
gagttgctagagacaaggagaaagaaa

SEQ ID NO: 19 - DNA, 24 bp, Arabidopsis thaliana
  source 1..24: mol_type = other DNA
tcttgagaacaatcccaattcatc

SEQ ID NO: 20 - DNA, 25 bp, Arabidopsis thaliana
  source 1..25: mol_type = other DNA
cctaagctcacaccctaaatcatgt

SEQ ID NO: 21 - DNA, 24 bp, Arabidopsis thaliana
  source 1..24: mol_type = other DNA
tttggagaatagtcagaccagtgc

SEQ ID NO: 22 - DNA, 24 bp, Arabidopsis thaliana
  source 1..24: mol_type = other DNA
tcactataaagagaatcggaagca

SEQ ID NO: 23 - DNA, 25 bp, Arabidopsis thaliana
  source 1..25: mol_type = other DNA
taccgattggagaatgagcatctaa

SEQ ID NO: 24 - DNA, 29 bp, Arabidopsis thaliana
  source 1..29: mol_type = other DNA
ttagtcttcttcatccacttctctgaaag

SEQ ID NO: 25 - DNA, 21 bp, Arabidopsis thaliana
  source 1..21: mol_type = other DNA
gaggttaacgatgatgctgtc

SEQ ID NO: 26 - DNA, 20 bp, Arabidopsis thaliana
  source 1..20: mol_type = other DNA
cctggaaatctgaagaaggc